(19) United States
(12) Patent Application Publication
(10) Pub. No.: US 2009/0239928 A1
(43) Pub. Date: Sep. 24, 2009

(54) N-Pyrrolidin-3YL-Amide Derivatives As Serotonin and Noradrenalin Re-Uptake Inhibitors

(76) Inventors: Paul Vincent Fish, Kent (GB); Thomas Ryckmans, Kent (GB); Alan Stobie, Kent (GB); Florian Wakenhut, Kent (GB)

Correspondence Address: PFIZER INC.;PATENT DEPARTMENT, Bld 114 M/S 114, EASTERN POINT ROAD, GROTON, CT, 06340, US

(21) Appl. No.: 11/721,352

(22) PCT Filed: Dec. 2, 2005
(86) PCT No.: PCT/IB05/03751

§ 371 Date: Aug. 3, 2007

Related U.S. Application Data
(60) Provisional application No. 60/647,838, filed on Jan. 27, 2005.
(60) Provisional application No. 60/695,215, filed on Jun. 28, 2005.

(30) Foreign Application Priority Data
Dec. 14, 2004 (GB) 0427358.7

(51) Int. Cl. A61K 31/40; C07D 207/14; A61P 13/00

(52) U.S. Cl. 514/426; 548/557

(57) ABSTRACT

A compound of Formula (I) and pharmaceutically and/or veterinarily acceptable derivatives thereof, wherein: R¹ is H, C₁₋₆alkyl, —C(A)D, C₃₋₈cycloalkyl, aryl, het, aryl-C₁₋₄alkyl or het-C₁₋₄alkyl, wherein the cycloalkyl, aryl or het groups are optionally substituted by at least one substituent independently selected from C₁₋₈allkyl, C₁₋₈alkoxy, OH, halo, CF₃, OCHF₂, OCF3, SCF₃, hydroxy-C₁₋₆alkyl, C₁₋₄alkoxy-C₁₋₆alkyl and C₁₋₄alkyl-S—C₁₋₄alkyl; A is S or O; D is H, C₁₋₆alkyl, aryl, het, aryl-C₁₋₄alkyl or het-C₁₋₄alkyl; R² represents aryl¹ or het¹, each of which is substituted by at least one substituent independently selected from B, provided that when R² is substituted by halo then it is also substituted with at least one other substituent independently selected from B other than halo; B represents aryl², het², Oaryl², Ohet², Sarl², Shet², SC₁₋₆alkyl, halogen, CHF₂, OCHF₂, CF₂CF₃, CH₂CF₃, CF2CH₃, aryl²-C₁₋₄alkyl, C₃₋₆cycloalkyl, C₃₋₆cycloalkyl-C₁₋₄alkyl, C₃₋₆cycloalkylC₁₋₄alkoxy, C₃₋₆cycloalkyl-O—C₁₋₄ alkyl, C₃₋₆cycloalkyl-C₁₋₄alkoxy-C₁₋₄alkyl, OC₃₋₆cycloalkyl, SC₃₋₆cycloalkyl; wherein the aryl² and het² groups are optionally substituted by at least one group selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁$alkyl, C₁₋₄alkoxy-C₁₋₄alkyl, SC₁₋₆alkyl and SCF₃; n is 1 or 2, provided that when n is 1, m is 0 or 1 and when n is 2, m is 0, wherein if m is 0, then * represents a chiral centre; R³ is H, C₁₋₆alkyl, C₃₋₈cycloalkyl, C₃₋₈cycloalkylC₁₋₆alkyl, aryl³, het³, aryl³-C₁₋₄alkyl or het³-C₁₋₄alkyl, wherein the C₃₋₈cycloalkyl, aryl³ or het³ groups are optionally substituted by at least one substituent independently selected from C₁₋₆alkyl, C₁₋₆alkoxy, CN, OH, halo, CF₃, OCF₃, SCF₃, hydroxy-C₁₋₆alkyl, C₁₋₄alkoxy-C₁₋₆alkyl and C₁₋₄alkyl-S—C₁₋₄alkyl; at each occurrence aryl, aryl¹, aryl² and aryl³ independently represent phenyl, naphthyl, anthracyl or phenanthryl; het¹ represents an aromatic 5- or 6-membered heterocycle which contains at least one N, O or S heteroatom, optionally fused to an aryl group; at each occurrence het, het², and het³ independently represents an aromatic or non-aromatic 4-, 5- or 6-membered heterocycle which contains at least one N, O or S heteroatom, optionally fused to a 5- or 6-membered carbocyclic group or a second 4-, 5- or 6membered heterocycle which contains at least one N, O or S heteroatom.

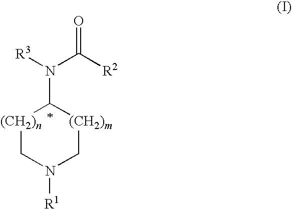

This invention relates to novel amide compounds which inhibit monoamine re-uptake, to processes for their preparation, to pharmaceutical compositions containing them and to their use in medicine.

The compounds of the invention exhibit activity as serotonin and/or noradrenaline re-uptake inhibitors and therefore have utility in a variety of therapeutic areas. For example, the compounds of the invention are of use in the treatment of disorders in which the regulation of monoamine transporter function is implicated, more particularly disorders in which inhibition of re-uptake of serotonin or noradrenaline is implicated. Furthermore, the compounds of the invention are of use in disorders in which inhibition of both serotonin and noradrenaline is implicated, such as urinary incontinence. Additionally, the compounds of the invention are of use in disorders in which it may be desired to inhibit preferentially the reuptake of one of noradrenaline or serotonin compared with the other, such as pain.

According to a first aspect, the invention provides a compound of formula (I),

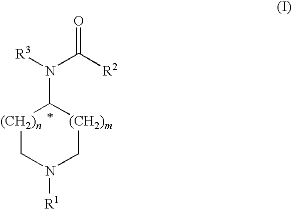

and pharmaceutically and/or veterinarily acceptable derivatives thereof, wherein:

R¹ is H, C₁₋₆alkyl, —C(A)D, C₃₋₈cycloalkyl, aryl, het, aryl-C₁₋₄alkyl or het-C₁₋₄alkyl, wherein the cycloalkyl, aryl or het groups are optionally substituted by at least one substituent independently selected from C₁₋₈allkyl, C₁₋₆alkoxy, OH, halo, CF₃, OCHF₂, OCF3, SCF₃, hydroxy-C₁₋₆alkyl, C₁₋₄alkoxy-C₁₋₆alkyl and C₁₋₄alkyl-S—C₁₋₄alkyl;
A is S or O;
D is H, C₁₋₆alkyl, aryl, het, aryl-C₁₋₄alkyl or het-C₁₋₄alkyl;
at each occurrence aryl independently represents phenyl, naphthyl, anthracyl or phenanthryl;
at each occurrence het independently represents an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom;

R² represents aryl¹ or het¹, each of which is substituted by at least one substituent independently selected from B, provided that when R² is substituted by halo then it is also substituted with at least one other substituent independently selected from B other than halo;
aryl¹ is selected from phenyl, naphthyl, anthracyl and phenanthryl;
het¹ represents an aromatic 5- or 6-membered heterocycle which contains at least one N, O or S heteroatom, optionally fused to an aryl group;
B represents aryl², het², Oaryl², Ohet², Saryl², Shet², SC₁₋₆alkyl, halogen, CHF₂, OCHF₂, CF₂CF₃, CH₂CF₃, CF₂CH₃, aryl²-C₁₋₄alkyl, C₃₋₆cycloalkyl, C₃₋₆cycloalkyl-C₁₋₄alkyl, C₃₋₆cycloalkyl-C₁₋₄alkoxy, C₃₋₆cycloalkyl-O—C₁₋₄alkyl, C₃₋₆cycloalkyl-C₁₋₄alkoxy-C₁₋₄alkyl, OC₃₋₆cycloalkyl, SC₃₋₆cycloalkyl; wherein the aryl² and het² groups are optionally substituted by at least one group selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃;
   at each occurrence, aryl² independently represents phenyl, naphthyl, anthracyl or phenanthryl;
   at each occurrence, het² independently represents an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom;

n is 1 or 2, provided that when n is 1, m is 0 or 1 and when n is 2, m is 0, wherein if m is 0, then * represents a chiral centre;

R³ is H, C₁₋₆alkyl, C₃₋₈cycloalkyl, C₃₋₈cycloalkyl-C₁₋₆alkyl, aryl³, het³, aryl³-C₁₋₄alkyl or het³-C₁₋₄alkyl, wherein the C₃₋₈cycloalkyl, aryl³ or het³ groups are optionally substituted by at least one substituent independently selected from C₁₋₆alkyl, C₁₋₄alkoxy, CN, OH, halo, CF₃, OCF₃, SCF₃, hydroxy-C₁₋₆alkyl, C₁₋₄alkoxy-C₁₋₆alkyl and C₁₋₄alkyl-S—C₁₋₄alkyl;
aryl³ represents phenyl, naphthyl, anthracyl or phenanthryl; and
het³ represents an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom.

In an embodiment of the invention, R¹ is H.

In a further embodiment of the invention, m is 0. Where m is 0, * represents the R or S enantiomeric configuration. Thus, in a further embodiment, m is 0; and * represents the S enantiomer. In a yet further embodiment, n is 1.

In a still further embodiment, aryl¹ represents phenyl or naphthyl. In a yet further embodiment, het¹ represents pyridinyl or quinolinyl. In a further embodiment, aryl¹ represents phenyl. In a still further embodiment het¹ represents pyridinyl. In a yet further embodiment, R² represents aryl¹. In a further embodiment, aryl² represents phenyl.

In a further embodiment, B represents Oaryl², SC₁₋₆alkyl, Saryl², C₁₋₄alkyl-aryl², halogen, OCHF₂, CF₂CH₃, C₃₋₆cycloalkyl, and C₃₋₆cycloalkyl-C₁₋₄alkyloxy, wherein the aryl² group is independently optionally substituted by at least one group independently selected from C₁₋₆alkyl I, C₃₋₆cycloalkyl, C₁₋₆alkoxy, OC₃₋₆cycloalkyl, halo, CN, OH, CF₃, CHF₂. OCF₃, OCHF₂, hydroxyC₁₋₆alkyl, C₁₋₄alkoxy-C₁₋₄alkyl, SC₁₋₆alkyl and SCF₃.

In a still further embodiment, B is Oaryl², SC₁₋₆alkyl, halogen, CF₂CH₃, and C₃₋₆cycloalkyl; wherein the aryl² group is independently optionally substituted by at least one group independently selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃; preferably C₁₋₆alkyl is C₁₋₄alkyl; preferably halogen and halo independently represent chloro or fluoro, preferably fluoro.

In a further embodiment, B is Oaryl², SC₁₋₆alkyl, CF₂CH₃, and C₃₋₆cycloalkyl; wherein the aryl² group is independently optionally substituted by at least one group independently selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃; preferably C₁₋₆alkyl is C₁₋₄alkyl; preferably halogen and halo independently represent chloro or fluoro, preferably fluoro.

In a yet further embodiment aryl² is optionally substituted by at least one group independently selected from C₁₋₆alkyl, and halo; preferably C₁₋₆alkyl represents C₁₋₄alkyl, more preferably it represents C₁₋₂alkyl, more preferably it represents methyl; preferably halo represents chloro or fluoro; even more preferably halo represents fluoro.

In a further embodiment, aryl¹ is optionally substituted with one or two independently selected substituents; preferably aryl¹ is optionally substituted with one substituent. In a further embodiment, aryl² is optionally substituted with one or two independently selected substituents; preferably aryl² is optionally substituted with one substituent.

In a yet still further embodiment, R³ is other than H.

In a yet still further embodiment, R³ represents C₁₋₆alkyl, C₃₋₈cycloalkyl, C₃₋₈cycloalkyl-C₁₋₆alkyl, aryl³ or het³, wherein the C₃₋₈cycloalkyl, aryl³ and het³ groups are independently optionally substituted by at least one substituent independently selected from: C₁₋₆-alkyl, C₁₋₆alkoxy, OH, halo, CN, CF₃, OCF₃, SCF₃, hydroxy-C₁₋₆alkyl, C₁₋₄alkoxy-C₁₋₆alkyl and C₁₋₄alkyl-S—C₁₋₄alkyl.

In a further embodiment R³ represents C₁₋₆alkyl, C₃₋₈cycloalkyl, C₃₋₆cycloalkyl-C₁₋₆alkyl, or aryl³, wherein the C₃₋₈cycloalkyl and aryl³ groups are independently optionally substituted by at least one substituent independently selected from C₁₋₆alkyl, C₁₋₆alkoxy, OH, halo, CF₃, OCF₃, SCF₃, hydroxy-C₁₋₆alkyl, C₁₋₄alkoxy-C₁₋₆alkyl and C₁₋₄alkyl-S—C₁₋₄alkyl.

In a yet further embodiment C₃₋₆cycloalkyl is independently optionally substituted by at least one substituent independently selected from C₁₋₆alkyl and CF₃; preferably the C₃₋₆cycloalkyl group is optionally substituted by at least one substituent independently selected from C₁₋₆alkyl, preferably methyl.

In a further embodiment aryl³ is selected from phenyl or napthyl, preferably it is phenyl. In a further embodiment, aryl³ is optionally substituted with one or more substituents independently selected from methyl, CF₃, or halo; preferably halo; preferably chloro or fluoro, more preferably fluoro.

In yet a further embodiment, het³ represents tetrahydropyranyl.

In an alternative aspect of the invention, there is provided a compound of formula I:

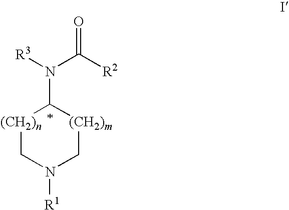

and pharmaceutically and/or veterinarily acceptable derivatives thereof, wherein:

R¹ is H, C₁₋₆alkyl, —C(A)D, C₃₋₈cycloalkyl, aryl, het, aryl-C₁₋₄alkyl or het-C₁₋₄alkyl, wherein the cycloalkyl, aryl or het groups are optionally substituted by at least one substituent independently selected from C₁₋₈allkyl, C₁₋₈alkoxy, OH, halo, CF₃, OCHF₂, OCF3, SCF₃, hydroxy-C₁₋₆alkyl, C₁₋₄alkoxy-C₁₋₆alkyl and C₁₋₄alkyl-S—C₁₋₄alkyl;

R² is aryl or heteroaryl, each of which is substituted by at least one substituent independently selected from aryl¹, het¹, Oaryl¹, Ohet¹, Saryl¹, Shet¹, CHF₂, OCHF₂, CF₂CF₃, CH₂CF₃. CF₂CH₃, C₃₋₆cycloalkyl, C₃₋₆cycloalkyl-C₁₋₄alkyl, C₃₋₆cycloalkyl-C₁₋₄alkoxy, C₃₋₆cycloalkyl-O—C₁₋₄alkyl, C₃₋₆cycloalkyl-C₁₋₄alkoxy-C₁₋₄alkyl, OC₃₋₆cycloalkyl, SC₃₋₆cycloalkyl, wherein the aryl¹ and het¹ groups are optionally substituted by at least one group selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃;

A is S or O;

D is H, C₁₋₆alkyl, aryl, het, aryl-C₁₋₄alkyl or het-C₁₋₄alkyl;

n is 1 or 2, provided that when n is 1, m is 0 or 1 and when n is 2, m is 0, wherein if m is 0, then * represents a chiral centre;

R³ is C₁₋₆alkyl, C₃₋₈cycloalkyl, C₃₋₈cycloalkyl-C₁₋₆alkyl, aryl, het, aryl-C₁₋₄alkyl or het-C₁₋₄alkyl, wherein the cycloalkyl, aryl or het groups are optionally substituted by at least one substituent independently selected from C₁₋₆alkyl, C₁₋₆alkoxy, OH, halo, CF₃, OCF₃, SCF₃, hydroxy-C₁₋₆alkyl, C₁₋₄alkoxy-C₁₋₆alkyl and C₁₋₄alkyl-S—C₁₋₄alkyl;

aryl and aryl¹ are each independently selected from phenyl, naphthyl, anthracyl and phenanthryl;

heteroaryl is an aromatic 5- or 6-membered heterocycle which contains at least one N, O or S heteroatom, optionally fused to an aryl group; and

het and het¹ are each independently selected from an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom.

In an embodiment of the invention, R¹ is H and R², R³, n and m are as defined above.

In a further embodiment of the invention, m is 0 and n, R¹, R² and R³ are as defined above. Where m is 0, * represents the R or S enantiomeric configuration. Thus, in a further embodiment, m is 0; n, R¹, R² and R³ are as defined above; and * represents the S enantiomer.

In a still further embodiment, R¹, R³, n and m are as defined above and R² is phenyl, naphthyl or quinolinyl, each substituted by at least one substituent independently selected from aryl¹, het¹, Oaryl¹, Ohet¹, Saryl¹, Shet¹, CHF₂, OCHF₂, CF₂CF₃, CH₂CF₃, CF₂CH₃, C₃₋₆cycloalkyl, C₃₋₆cycloalkyl-C₁₋₄alkyl, C₃₋₆cycloalkyl-C₁₋₄alkoxy, C₃₋₆cycloalkyl-O—C₁₋₄alkyl, C₃₋₆cycloalkyl-C₁₋₄alkoxy-C₁₋₄alkyl, OC₃₋₆cycloalkyl, SC₃₋₆cycloalkyl, wherein the aryl¹ and het¹ groups are optionally substituted by at least one group selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃.

In a yet further embodiment of the invention, there is provided a compound of Formula II

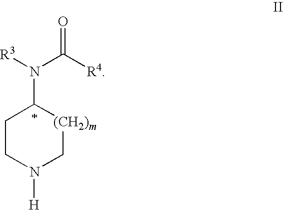

and pharmaceutically and/or veterinarily acceptable derivatives thereof, wherein:

R³ is as defined above;

R⁴ is phenyl, naphthyl, or quinolinyl, each substituted by at least one substituent independently selected from aryl¹, het¹, Oaryl¹, Ohet¹, Saryl¹, Shet¹, SC₁₋₆alkyl, halogen, CHF₂, OCHF₂, CF₂CF₃, CH₂CF₃, CF₂CH₃, C₃₋₆cycloalkyl, C₃₋₆cycloalkyl-C₁₋₄alkyl, C₃₋₆cycloalkyl-C₁₋₄alkoxy, C₃₋₆cycloalkyl-O—C₁₋₄alkyl, C₃₋₆cycloalkyl-C₁₋₄alkoxy-C₁₋₄alkyl, OC₃₋₆cycloalkyl, SC₃₋₆cycloalkyl, wherein the aryl¹ and het¹ groups are optionally substituted by at least one group selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃; and

m is 0 or 1, wherein if m is 0, then * represents the R or S enantiomer.

In a further embodiment, R⁴ is phenyl, 1-naphthyl or 2-naphthyl, each substituted by at least one substituent independently selected, from aryl¹, het¹, Oaryl¹, Ohet¹, Saryl¹, Shet¹, SC₁₋₆alkyl, halogen, CHF₂, OCHF₂, CF₂CF₃, CH₂CF₃, CF₂CH₃, C₃₋₆cycloalkyl, C₃₋₆cycloalkyl-C₁₋₄alkyl, C₃₋₆cycloalkyl-C₁₋₄alkoxy, C₃₋₆cycloalkyl-O—C₁₋₄alkyl, C₃₋₆cycloalkyl-C₁₋₄alkoxy-C₁₋₄alkyl, OC₃₋₆cycloalkyl, SC₃₋₆cycloalkyl, wherein the aryl¹ and het¹ groups are optionally substituted by at least one group selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl. C₁₋₄alkoxy-C₁₋₄alkyl, SC₁₋₆alkyl and SCF₃. The phenyl or naphthyl groups may be substituted by one, two or three substituents.

In a yet still further embodiment, m is 0. In this embodiment, * represents the R or S enantiomer. In a further embodiment, m is 0 and * represents the S enantiomer.

In a still further embodiment, there is provided a compound of Formula III

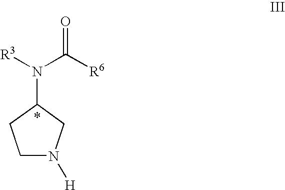

and pharmaceutically and/or veterinarily acceptable derivatives thereof,

wherein:

R³ is as defined above;

R⁶ is phenyl, naphthyl or quinolinyl, each substituted by at least one substituent independently selected from aryl¹, het¹, Oaryl¹, Ohet¹, Saryl¹, Shet¹, SC₁₋₆alkyl, halogen, CHF₂, OCHF₂, CF₂CF₃, CH₂CF₃, CF₂CH₃, C₃₋₆cycloalkyl, C₃₋₆cycloalkyl-C₁₋₄alkyl, C₃₋₆cycloalkyl-C₁₋₄alkoxy, C₃₋₆cycloalkyl-O—C₁₋₄alkyl, C₃₋₆cycloalkyl-C₁₋₄alkoxy-C₁₋₄alkyl, OC₃₋₆cycloalkyl, SC₃₋₆cycloalkyl, wherein the aryl¹ and het¹ groups are optionally substituted by at least one group selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃; and

* represents the R or S enantiomer.

In a further embodiment, R⁶ is phenyl, 1-naphthyl or 2-naphthyl, each substituted by at least one substituent independently selected from aryl¹, het¹, Oaryl¹, Ohet¹, Saryl¹, Shet¹, SC₁₋₆alkyl, halogen, CHF₂, OCHF₂, CF₂CF₃, CH₂CF₃₁ CF₂CH₃, C₃₋₆cycloalkyl, C₃₋₆cycloalkyl-C₁₋₄alkyl, C₃₋₆cycloalkyl-C₁₋₄alkoxy, C₃₋₆cycloalkyl-O—C₁₋₄alkyl, C₃₋₆cycloalkyl-C₁₋₄alkoxy-C₁₋₄alkyl, OC₃₋₆cycloalkyl, SC₃₋₆cycloalkyl, wherein the aryl¹ and het¹ groups are optionally substituted by at least one group selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃.

In a yet further embodiment, * represents the S enantiomer.

In a further embodiment, the invention provides a compound selected from:
N-ethyl-2-phenoxy-N-[(3S)-pyrrolidin-3-yl]benzamide;
N-isobutyl-2-phenoxy-N-[(3S)-pyrrolidin-3-yl]benzamide;
N-cyclobutyl-2-phenoxy-N-[(3S)-pyrrolidin-3-yl]benzamide;
N-cyclopentyl-2-phenoxy-N-[(3S)-pyrrolidin-3-yl]benzamide;
2-phenoxy-N-propyl-N-[(3S)-pyrrolidin-3-yl]benzamide;
N-isobutyl-2-phenoxy-N-[(3S)-pyrrolidin-3-yl]nicotinamide;
5-fluoro-2-phenoxy-N-propyl-N-[(3S)-pyrrolidin-3-yl]benzamide;
N-(cyclobutylmethyl)-2-(ethylthio)-N-[(3S)-pyrrolidin-3-yl]benzamide;
N-[(1-methylcyclobutyl)methyl]-2-(methylthio)-N-[(3S)-pyrrolidin-3-yl]benzamide;
2-(ethylthio)-N-[(1-methylcyclobutyl)methyl]-N-[(3S)-pyrrolidin-3-yl]benzamide;
2-(ethylthio)-N-[(1-methylcyclopropyl)methyl]-N-[(3S)-pyrrolidin-3-yl]benzamide;
N-(sec-butyl)-2-phenoxy-N-[(3S)-pyrrolidin-3-yl]benzamide;
N-(cyclobutylmethyl)-2-phenoxy-N-[(3S)-pyrrolidin-3-yl]benzamide;
2-phenoxy-N-phenyl-N-[(3S)-pyrrolidin-3-yl]benzamide;
N-(cyclobutylmethyl)-2-(methylthio)-N-[(3S)-pyrrolidin-3-yl]benzamide;
N-(cyclobutylmethyl)-2-(1,1-difluoroethyl)-N-[(3S)-pyrrolidin-3-yl]benzamide;
2-(ethylthio)-N-isobutyl-N-[(3S)-pyrrolidin-3-yl]benzamide;
N-(cyclopropylmethyl)-2-(ethylthio)-N-[(3S)-pyrrolidin-3-yl]benzamide;
2-cyclopentyl-N-(cyclopropylmethyl)-N-[(3S)-pyrrolidin-3-yl]benzamide;
N-cyclohexyl-2-(methylthio)-N-[(3S)-pyrrolidin-3-yl]benzamide;
N-(cyclobutylmethyl)-2-(isopropylthio)-N-[(3S)-pyrrolidin-3-yl]benzamide;
N-cyclopentyl-2-(ethylthio)-N-[(3S)-pyrrolidin-3-yl]benzamide;
N-cyclopentyl-2-(methylthio)-N-[(3S)-pyrrolidin-3-yl]benzamide;
N-(cyclopropylmethyl)-2-phenoxy-N-[(3S)-pyrrolidin-3-yl]benzamide;
N-[(1-methylcyclopropyl)methyl]-2-phenoxy-N-[(3S)-pyrrolidin-3-yl]benzamide;
N-cyclohexyl-2-phenoxy-N-[(3S)-pyrrolidin-3-yl]benzamide;
2-phenoxy-N-[(3S)-pyrrolidin-3-yl]-N-(tetrahydro-2H-pyran-4-yl)benzamide;
2-phenoxy-N-[(3S)-pyrrolidin-3-yl]-N-{[1-(trifluoromethyl)cyclopropyl]methyl}benzamide;
N-cyclobutyl-2-(difluoromethoxy)-N-[(3S)-pyrrolidin-3-yl]benzamide;
2-(difluoromethoxy)-N-[(1-methylcyclopropyl)methyl]-N-[(3S)-pyrrolidin-3-yl]benzamide;
N-cyclobutyl-3-phenoxy-N-[(3S)-pyrrolidin-3-yl]benzamide;
2-benzyl-N-isobutyl-N-[(3S)-pyrrolidin-3-yl]benzamide;
N-cyclobutyl-2-(methylthio)-N-[(3S)-pyrrolidin-3-yl]benzamide;
N-methyl-2-phenoxy-N-[(3S)-pyrrolidin-3-yl]benzamide;
N-cyclobutyl-2-(ethylthio)-N-[(3S)-pyrrolidin-3-yl]benzamide;
N-cyclobutyl-2-cyclopentyl-N-[(3S)-pyrrolidin-3-yl]benzamide;
2-cyclobutyl-N-cyclopentyl-N-[(3S)-pyrrolidin-3-yl]benzamide;
2-cyclobutyl-N-(2,2-dimethylpropyl)-N-[(3S)-pyrrolidin-3-yl]benzamide;
N,2-dicyclopentyl-N-[(3S)-pyrrolidin-3-yl]benzamide;
2-cyclopentyl-N-propyl-N-[(3S)-pyrrolidin-3-yl]benzamide;
2-cyclopentyl-N-(2,2-dimethylpropyl)-N-[(3S)-pyrrolidin-3-yl]benzamide;
N-isopropyl-2-phenoxy-N-[(3S)-pyrrolidin-3-yl]benzamide;
N-(cyclobutylmethyl)-2-cyclopropyl-N-[(3S)-pyrrolidin-3-yl]benzamide;
N-(cyclopropylmethyl)-2-(methylthio)-N-[(3S)-pyrrolidin-3-yl]benzamide;
2-cyclobutyl-N-(cyclopropylmethyl)-N-[(3S)-pyrrolidin-3-yl]benzamide;
2-cyclopropyl-N-(2,2-dimethylpropyl)-N-[(3S)-pyrrolidin-3-yl]benzamide;
N-isobutyl-2-(methylthio)-N-[(3S)-pyrrolidin-3-yl]benzamide;
N-(cyclobutylmethyl)-2-cyclopentyl-N-[(3S)-pyrrolidin-3-yl]benzamide;
2-cyclobutyl-N-(cyclobutylmethyl)-N-[(3S)-pyrrolidin-3-yl]benzamide;
N-(cyclobutylmethyl)-2-(difluoromethoxy)-N-[(3S)-pyrrolidin-3-yl]benzamide;
N-(cyclopentylmethyl)-2-(methylthio)-N-[(3S)pyrrolidin-3-yl]benzamide;
N-(cyclopentylmethyl)-2-cyclopropyl-N-[(3S)-pyrrolidin-3-yl]benzamide;
2-(methylthio)-N-propyl-N-[(3S)-pyrrolidin-3-yl]benzamide;
N-(2,2-dimethylpropyl)-2-(ethylthio)-N-[(3S)-pyrrolidin-3-yl]benzamide;
2-(ethylthio)-N-propyl-N-[(3S)-pyrrolidin-3-yl]benzamide;
N-(cyclopropylmethyl)-2-(isopropylthio)-N-[(3S)-pyrrolidin-3-yl]benzamide;
2-(methylthio)-N-phenyl-N-[(3S)-pyrrolidin-3-yl]benzamide;
3-fluoro-2-phenoxy-N-propyl-N-[(3S)-pyrrolidin-3-yl]benzamide;
2-(ethylthio)-N-phenyl-N-[(3S)-pyrrolidin-3-yl]benzamide;
N-(sec-butyl)-2-(ethylthio)-N-[(3S)-pyrrolidin-3-yl]benzamide;
N-(cyclobutylmethyl)-2-phenoxy-N-[(3R)-pyrrolidin-3-yl]benzamide;
N-ethyl-2-(4-fluorophenoxy)-N-[(3S)-pyrrolidin-3-yl]benzamide;
N-ethyl-5-fluoro-2-phenoxy-N-[(3S)-pyrrolidin-3-yl]benzamide;
2-(4-fluorophenoxy)-N-propyl-N-[(3S)-pyrrolidin-3-yl]benzamide;
N-(2-cyclopropylethyl)-2-(methylthio)-N-[(3S)-pyrrolidin-3-yl]benzamide;
N-(2-cyclopropylethyl)-2-(ethylthio)-N-[(3S)-pyrrolidin-3-yl]benzamide;
N-[(1-methylcyclopropyl)methyl]-2-(methylthio)-N-[(3S)-pyrrolidin-3-yl]benzamide;
N-cyclohexyl-2-(ethylthio)-N-[(3S)-pyrrolidin-3-yl]benzamide;
N-(2,2<dimethylpropyl)-2-(methylthio)-N-[(3S)-pyrrolidin-3-yl]benzamide;
N-cyclopentyl-2-cyclopropyl-N-[(3S)-pyrrolidin-3-yl]benzamide;
N-(sec-butyl)-2-(methylthio)-N-[(3S)-pyrrolidin-3-yl]benzamide;
N-cyclopropyl-2-phenoxy-N-pyrrolidin-3-yl]benzamide;
2-(4-chlorophenoxy)-N-propyl-N-[(3S)-pyrrolidin-3-yl]benzamide;
N-cyclobutyl-2-(phenylthio)-N-[(3S)-pyrrolidin-3-yl]benzamide;
N-(cyclobutylmethyl)-2-(cyclopropylmethoxy)-N-[(3S)-pyrrolidin-3-yl]benzamide;
   and a pharmaceutically and/or veterinarily acceptable derivative thereof.

The above described embodiments of the invention may be combined with one or more further embodiments such that further embodiments are provided wherein two or more variables are defined more specifically in combination. For example, within the scope of the invention is a further embodiment wherein the variables R¹, R², R³, R⁴, R⁶, m and n all have the more limited definitions assigned to them in the more specific embodiments described above. All such combinations of the more specific embodiments described and defined above are within the scope of the invention

By pharmaceutically and/or veterinarily acceptable derivative it is meant any pharmaceutically or veterinarily acceptable salt, solvate, ester or amide, or salt or solvate of such ester or amide, complex, polymorph, stereoisomer, geometric isomer, tautomeric form, or isotopic variation, of the compounds of formula (I), (II) or (III) or any other compound which upon administration to the recipient is capable of providing (directly or indiredly) a compound of formula (I), (II) or (III) or an active metabolite or residue thereof. Preferably, pharmaceutically acceptable derivatives are salts, solvates, esters and amides of the compounds of formula (I), (II) or (III). More preferably, pharmaceutically acceptable derivatives are salts and solvates.

For pharmaceutical or veterinary use, the salts referred to above will be the pharmaceutically or veterinarily acceptable salts, but other salts may find use, for example in the preparation of compounds of formula (I), (II) or (III) and the pharmaceutically or veterinarily acceptable salts thereof.

The aforementioned pharmaceutically or veterinarily acceptable salts include the acid addition and base salts thereof.

Suitable acid addition salts are formed from acids which form non-toxic salts. Examples include the acetate, aspartate, benzoate, besylate, bicarbonate/carbonate, bisulphate/sulphate, camsylate, citrate, hemicitrate, edisylate, hemiedisylate, esylate, fumarate, gluceptate, gluconate, glucuronate, hibenzate, hydrochloride/chloride, hydrobromide/bromide, hydroiodide/iodide, isethionate, lactate, malate, maleate, malonate, mesylate, methylsulphate, 2-napsylate, nicotinate, nitrate, orotate, pamoate, phosphate/hydrogen phosphate/dihydrogen phosphate, saccharate, stearate, succinate, tartrate and tosylate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of a compound of formula (I), (II) or (III) may be readily prepared by mixing together solutions of the compound and the desired acid or base, as appropriate. The salt may precipitate from solution and be collected by filtration or may be recovered by evaporation of the solvent. The degree of ionisation in the salt may vary from completely ionised to almost non-ionised.

Pharmaceutically acceptable solvates in accordance with the invention include hydrates and solvates of the compounds of formula (I), (II) or (III).

Also within the scope of the invention are complexes such as clathrates, drug-host inclusion complexes wherein, in contrast to the aforementioned solvates, the drug and host are present in stoichiometric or non-stoichiometric amounts. Also included in this invention are complexes of the pharmaceutical drug which contain two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

The compounds of formula (I), (II) or (III) may be modified to provide pharmaceutically or veterinarily acceptable derivatives thereof at any of the functional groups in the compounds. Examples of such derivatives are described in: Drugs of Today, Volume 19, Number 9,1983, pp 499-538; Topics in Chemistry, Chapter 31, pp 306-316; and in “Design of Prodrugs” by H. Bundgaard, Elsevier, 1985, Chapter 1 (the disclosures in which documents are incorporated herein by reference) and include: esters, carbonate esters, hemi-esters, phosphate esters, nitro esters, sulfate esters, sulfoxides, amides, sulphonamides, carbamates, azo-compounds, phosphamides, glycosides, ethers, acetals and ketals.

It will be further appreciated by those skilled in the art, that certain moieties, known in the art as “pro-moieties”, for example as described by H. Bundgaard in “Design of Prodrugs” (ibid) may be placed on appropriate functionalities when such functionalities are present within compounds of the invention.

The compounds of formula (I), (II) or (III) may contain one or more chiral centres, by virtue of the asymmetric carbon atom defined by certain meanings of R¹ to R⁹ (e.g. s-butyl), or the value of the integer m. Such compounds exist in a number of stereoisomeric forms (e.g. in the form of a pair of optical isomers, or enantiomers). It is to be understood that the present invention encompasses all isomers of the compounds of the invention, including all geometric, tautomeric and optical forms, and mixtures thereof (e.g. tautomeric or racemic mixtures).

The compounds of the invention may exist in one or more tautomeric forms. All tautomers and mixtures thereof are included in the scope of the present invention. For example, a claim to 2-hydroxypyridinyl would also cover its tautomeric form α-pyridonyl.

It is to be understood that the present invention includes radio labelled compounds of formula (I), (II) or (III).

The compounds of formula (I), (II) or (III) and their pharmaceutically and veterinarily acceptable derivatives thereof may also be able to exist in more than one crystal form, a characteristic known as polymorphism. All such polymorphic forms (“polymorphs”) are encompassed within the scope of the invention. Polymorphism generally can occur as a response to changes in temperature or pressure or both, and can also result from variations in the crystallisation process. Polymorphs can be distinguished by various physical characteristics, and typically the x-ray diffraction patterns, solubility behaviour, and melting point of the compound are used to distinguish polymorphs.

Unless otherwise indicated, any alkyl group may be straight or branched and is of 1 to 8 carbon atoms, such as 1 to 6 carbon atoms or 1 to 4 carbon atoms, for example a methyl, ethyl, n-propyl, i-propyl, n-butyl, i-butyl, s-butyl or t-butyl group. Where the alkyl group contains more than one carbon atom, it may be unsaturated. Thus, the term C₁₋₆ alkyl includes C₂₋₆ alkenyl and C₂₋₆ alkynyl. Similarly, the term C₁₋₈alkyl includes C₂₋₈ alkenyl and C₂₋₈ alkynyl, and the term C₁₋₄ alkyl includes C₂₋₄ alkenyl and C₂₋₄ alkynyl.

The term halogen is used to represent fluorine, chlorine, bromine or iodine.

Unless otherwise indicated, the term het includes any aromatic, saturated or unsaturated 4-, 5- or 6-membered heterocycle which contains up to 4 heteroatoms selected from N, O and S. Examples of such heterocyclic groups included furyl, thienyl, pyrrolyl, pyrrolinyl, pyrrolidinyl, imidazolyl, dioxolanyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, thiadiazolyl, pyranyl, tetrahydropyranyl, pyridyl, piperidinyl, dioxanyl, morpholino, dithianyl, thiomorpholino, pyridazinyl, pyrimidinyl, pyrazinyl, piperazinyl, sulfolanyl, tetrazolyl, triazinyl, azepinyl, oxazapinyl, thiazepinyl, diazepinyl and thiazolinyl. In addition, the term heterocycle includes fused heterocyclyl groups, for example benzimidazolyl, benzoxazolyl, imidazopyridinyl, benzoxazinyl, benzothiazinyl, oxazolopyridinyl, benzofuranyl, quinolinyl, quinazolinyl, quinoxalinyl, dihydroquinazdinyl, benzothiazolyl, phthalimido, benzodiazepinyl, indolyl and isoindolyl. The terms het, heterocyclyl and heterocyclic should be similarly construed.

For the avoidance of doubt, unless otherwise indicated, the term substituted means substituted by one or more defined groups. In the case where groups may be selected from a number of alternative groups, the selected groups may be the same or different. Further, the term independently means that where more than one substituent is selected from a number of possible substituents, those substituents may be the same or different.

Hereinafter, the compounds of formula (I), (II) or (III) and their pharmaceutically and veterinarily acceptable derivatives, the radio-labelled analogues of the foregoing, the isomers of the foregoing, and the polymorphs of the foregoing, are referred to as “compounds of the invention”.

In one embodiment of the invention, the compounds of the invention are the pharmaceutically and veterinarily acceptable derivatives of compounds of formula (I), (II) or (III), such as the pharmaceutically or veterinarily acceptable salts or solvates of compounds of formula (I), (II) or (III), (e.g. pharmaceutically or veterinarily acceptable salts of compounds of formula (I), (II) or (III)).

In a still further embodiment of the invention, there is provided a compound of the invention which is an inhibitor of serotonin and/or noradrenaline monoamine re-uptake, having SRI or NRI Ki values of 500 nM or less, preferably 200 nM or less. In a further embodiment, the compound has SRI and/or NRI Ki values of 100 nM or less. In a yet further embodiment, the compound has SRI and/or NRI Ki values of 50 nM or less. In a still yet further embodiment, the compound has SRI and/or NRI Ki values of 25 nM or less.

Without wishing to be bound by theory, it is believed that for certain of the diseases or conditions for which the compounds of the invention are indicated it is useful for the compound to be a more potent inhibitor of the reuptake of one of serotonin or noradrenaline than the other. Thus, in an embodiment of the invention, the reuptake of noradrenaline is inhibited to greater degree than the reuptake of serotonin. In an alternative embodiment, the reuptake of serotonin is inhibited to a greater degree than the reuptake of noradrenaline. For example, in the treatment of pain, it is believed that compounds of the invention which inhibit the reuptake of noradrenaline have good efficacy. Thus, an embodiment of the invention provides a method of treating pain which comprises administering to a patient in need of such treatment a therapeutically effective amount of a compound according to the invention which is capable of inhibiting the reuptake of noradrenaline. In this embodiment, the compound of the invention may selectively inhibit the reuptake of noradrenaline or it may inhibit the reuptake of noradrenaline preferentially to the inhibition of serotonin reuptake or it may inhibit the reuptake of serotonin preferentially to the inhibition of noradrenaline reuptake. In a further embodiment of the invention, there provided compounds which are more potent noradrenalin reuptake inhibitors than serotonin reuptake inhibitors. Accordingly, such an embodiment of the invention provides a method of treating pain which comprises administering to a patient in need of such treatment a therapeutically effective amount of a compound according to the invention which is capable of inhibiting the reuptake of noradrenaline to a greater extent than the reuptake of serotonin.

According to Scheme 1, compounds of Formula (V) may be prepared from compounds of Formula (VI) by reaction with an aldehyde R³CHO or a suitable ketone, followed by reaction with an acid or acid chloride R²COX (where X is OH or halo), or an acid anhydride, optionally further followed by deprotection.

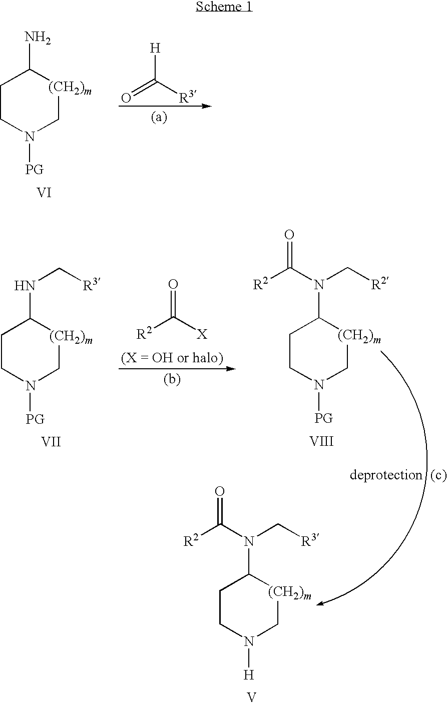

In the above scheme, R² and m are as defined above, PG is a protecting group and the moiety —CH₂R³ satisfies the definition of R³.

(a)-Reductive Amination

The reaction of the 1° amine (VI) with the aldehyde to form the 2° amine (VII) is a reductive amination reaction, in which the dehydration of the amine and the aldehyde is followed by reduction of the formed imine by a metal hydride reagent or hydrogenation, in a suitable solvent at room temperature.

In this reaction, equimolar amounts of amine and aldehyde are typically treated with either sodium triacetoxyborohydride (STAB), NaCN(BH)₃ or NaBH₄, in a suitable solvent (e.g. DCM, THF) at room temperature for 1 to 24 hours. Alternatively, an excess of a reducing agent (e.g. NaBH₄, LiAlH₄, STAB) in a suitable solvent (e.g. THF. MeOH, EtOH) is added after the amine and aldehyde have been mixed for 1-18 hours, optionally in the presence of a drying agent (e.g. molecular sieve) or with the removal of water using Dean-Stark apparatus with a suitable solvent (e.g. toluene, xylene). A further alternative involves catalytic hydrogenation in the presence of a palladium or nickel catalyst (e.g. Pd/C, Raney® Ni) under an atmosphere of H₂, optionally at elevated temperature and pressure, in a suitable solvent (e.g. EtOH).

A more specific example of the reductive amination involves treatment of the aldehyde with the amine in the presence of either 10% Pd/C, optionally in the presence of triethylamine, in ethanol under about 415 kPa (about 60 psi) of hydrogen at room temperature for 18 hours, or an excess of sodium borohydride in methanol and toluene at room temperature for 18 hours.

(b)-Amide Formation

The formation of a peptide linkage between the acid, acid halide or other suitable carbonyl-containing group and the amine (VII) may be undertaken by using either:

(i) the acid halide (or acid or anhydride) and the amine (VII), with an excess of acid acceptor in a suitable solvent, or

(ii) the acid, optionally with a conventional coupling agent, and the amine (VII), optionally in the presence of a catalyst, with an excess of acid acceptor in a suitable solvent.

Examples of such reactions are as follows:
(a) An acid chloride (optionally generated in-situ) is reacted with an excess of the amine (VII), optionally with an excess of 3° amine such as Et₃N, Hünig's base or NMM, in DCM, dioxane or toluene optionally in the presence of DMAP, optionally at elevated temperature for 1 to 24 hrs;
(b) An acid, WSCDI/DCCI/TBTU and HOBT/HOAT is reacted with an excess of amine (VII) and an excess of NMM, Et₃N, Hünig's base in THF, DCM or EtOAc, at rt. for 4 to 48 hrs; or
(c) An acid and PYBOP®/PyBrOP®/Mukaiyama's reagent is reacted with an excess of amine (VII) and an excess of NMM, Et₃N, Hünig's base in THF, DCM or EtOAc, at rt. for 4 to 24 hrs.

Where the acid halide is an acid chloride (i.e. X═Cl), this may be generated in-situ by standard methodology and then reacted with the amine (VII) in the presence of triethylamine in dichloromethane at 70° C. for 90 minutes.

(c)-Deprotection

Where PG is a suitable amine-protecting group, preferably BOC, triftuoroacetate, benzyloxycarbonyl or benzyl, the removal of PG from (VIII), to form the unprotected amine (V), is performed by a method selective to the protecting group as detailed in “Protective Groups in Organic Synthesis”, 3^{rd} edition, by TW Greene and PGM Wuts. John Wiley and Sons, Inc. 1999, incorporated herein by reference.

Examples of such deprotection reactions are as follows:

When PG is BOC, the deprotection involves treatment of (VIII) with an excess of strong acid (e.g. HCl, TFA) at room temperature in a suitable solvent (e.g. DCM, EtOAc, dioxan).

When PG is trifluoroacetate, the deprotection involves treatment of (VIII) with a base (e.g. K₂CO₃, Na₂CO₃, NH₃, Ba(OH)₂) in an alcoholic solvent (e.g. MeOH, EtOH), optionally with water and optionally at elevated temperature.

When PG is Bh or Bz, the deprotection involves either transfer hydrogenation with a transition metal or transition metal salt hydrogenation catalyst (e.g. Pd/C, Pd(OH)₂) in the presence of a hydrogen donor (e.g. NH₄⁺HCO₂⁻) in a polar solvent (e.g. tetrahydrofuran, ethanol, methanol) optionally at elevated temperature and/or pressure, or catalytic hydrogenation in the presence of a palladium or nickel catalyst (e.g. Pd/C, Raney® Ni) under an atmosphere of H₂, optionally at elevated temperature and pressure, in a suitable solvent.

More specifically:

When PG is BOC, the deprotection involves treatment with either an excess of 4M hydrochloric acid in dioxan for up to 18 hours at room temperature, or with TFA in DCM for about 4.5 hours at RT. When PG is trifluoroacetate, the deprotection involves treatment with K₂CO₃ in methanol:water mixture (5:1 to 10:1) at room temperature for 18 hours.

When PG is Bn or Bz, the deprotection involves treatment with NH₄⁺HCO₂⁻ and 10% Pd/C in ethanol under gentle reflux for between 6 and 20 hours.

Alternative methods for preparing the secondary amine compound (VII) from the primary amine compound (VI) are described in Schemes 1a and 1b below.

In Schemes 1a and 1b, the value of m is shown as 0 and the value of n is 1. However, it will be immediately apparent to those skilled in the art that corresponding reagents, where m is 1 or where n is 2, can be used in the following schemes to prepare the appropriate end compounds.

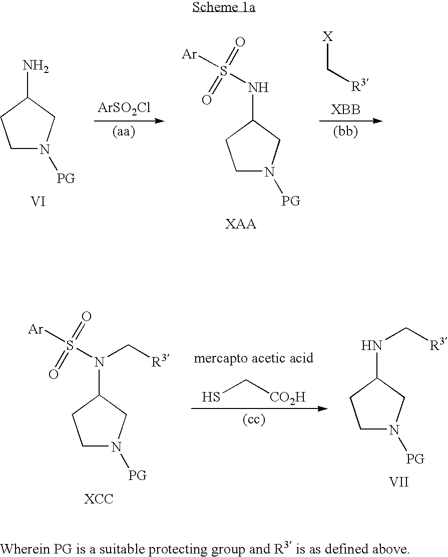

According to Scheme 1a, compounds of formula VII can be prepared from compounds of formula VI by reaction with a sulfonyl chloride, followed by alkylation of the resulting sulfonyl amide, and then removal of the sulfonyl moiety.

### (aa) Preparation of the Sulfonyl Amide.

Reaction of equimolar amounts of the primary amine (VI) and a sulfonyl chloride such as 4-nitrobenzenesulfonyl chloride or 2,4-dinitrobenzenesulphonyl chloride in a suitable solvent (such as DCM, THF or Toluene) in the presence of an organic base (such as pyridine, 2,6-lutidine or Hünig's base) or an inorganic base (such as a carbonate salt) for up to 24 hours affords the sulfonylamide (XAA).

### (bb) Alkylation of Sulfonylamide XAA.

The sulfonylamide of formula XAA is alkylated using an activated alkylating agent XBB, where X is a leaving group such as a halogen (e.g. iodo, bromo or chloro) or a sulfonyl ester (such as methanesulfonate) in the presence of an organic or an inorganic base, in a suitable solvent (such as DMF or THF). Alternatively the alkylation of sulfonylamide of formula XAA can be achieved using an alcohol XBB (where X is OH), a phosphine (such as triphenyl phosphine) and an azodicarboxylate compound (such as DIAD) in a suitable solvent, such as THF, for up to 24 hours at a temperature between −20° C. and 45° C.

### (cc) Removal of the Sulfonyl Group.

A compound of formula XCC is treated with an organic base (such as triethylamine) or an inorganic base (such as a carbonate or a hydroxide, e.g. LiOH) in a suitable solvent (such as DCM, THF, DMF or a lower alcohol) and with a thiol (such as mercaptoacetic acid) for up to 24 hours, optionally at an elevated temperature.

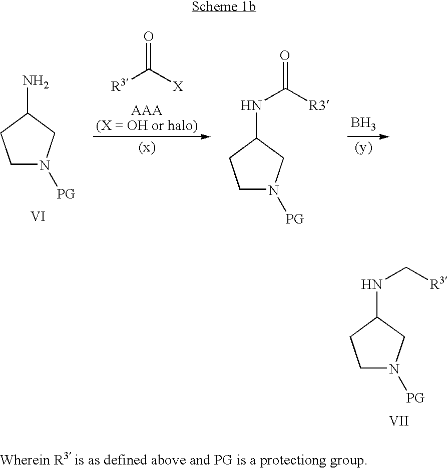

### Acylation-Reduction

According to Scheme 1b, compounds of Formula (VII) may be prepared from 1° amine of Formula (VI) by reaction with a carboxylic acid, acid anhydride or acid halide AAA (optionally prepared in-situ) R⁴COX (where X is OH, OC(O)R^{3′} or halo), followed by reaction with a reducing agent, such as borane.

(x)-Amide Formation

The formation of an amide bond between the acid or acid halide and the 1° amine (VI) may be undertaken by using either:

(i) the acyl halide (or the acid or acid anhydride) and the amine (VI), with an excess of acid acceptor in a suitable solvent, or
(ii) the acid, optionally with a conventional coupling agent, and the amine (VI), optionally in the presence of a catalyst, with an excess of acid acceptor in a suitable solvent.

Examples of such reactions are as follows:
(a) An acid anhydride is reacted with an excess of the amine (VI), optionally with an excess of a 3°amine such as Et₃N, Hünig's base or NMM, in DCM or toluene, optionally at elevated temperature, for 1 to 72 hours.
(b) An acid chloride (optionally generated in-situ) is reacted with an excess of the amine (VI), optionally with an excess of 3° amine such as Et₃N, Hünig's base or NMM, in DCM or dioxane, optionally at elevated temperature for 1 to 24 hrs;
(c) An acid, WSCDI/DCCI/TBTU and HOBT/HOAT is reacted with an excess of amine (VI) and an excess of NMM, Et₃N, Hünig's base in THF, DCM or EtOAc, at rt. for 4 to 48 hrs; or
(d) An acid and 1-propyl phosphonic ester cyclic anhydride/PYBOP®/PyBrOP®/Mukaiyama's reagent is reacted with an excess of amine (VI) and an excess of NMM, Et₃N, Hünig's base in THF, DCM or EtOAc, at rt. for 1 to 24 hrs.

A more specific example of the amide formation involves treatment of the amine with a suitable acid anhydride in the presence of N-methylmorpholine in toluene at room temperature for 72 hours

Where the acid halide is an acid chloride (i.e. X═Cl), this may be generated in-situ by standard methodology and then reacted with the amine (VI) and triethylamine in dichloromethane at 70° C. for 90 minutes

(v)-Reduction

The reaction (y) is a reduction of the amide to amine (VII) for example by a hydride reducing agent under suitable conditions.

Conveniently, the reduction of the amide is carried out in the presence of Borane in THF at between room temperature and reflux for up to 48 hours, followed by addition of methanol and optionally of aqueous ammonium chloride and further reflux for 4-18 hours before isolation of the amine (VII).

According to Scheme 2, compounds of Formula (IX) may be prepared from compounds of Formula (VI) by reaction with R³-L, where L is a leaving group, under suitable conditions. The resulting compound of Formula (IX) may then be converted to a compound of Formula (II) by amide formation and deprotection in a manner analogous to that described above in relation to Scheme 1.

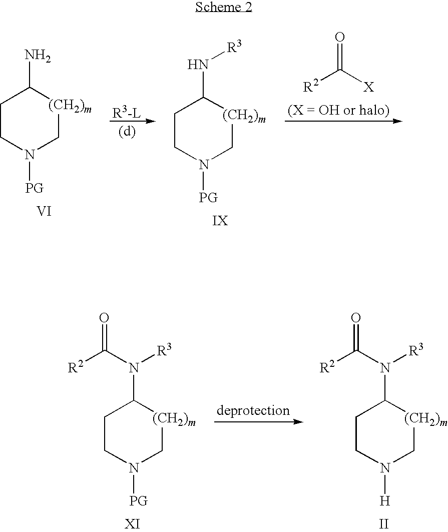

In the above scheme, R², R³ and m are as defined above, PG is a suitable protecting group and L is a leaving group, whose meaning will depend, inter alia, on the nature of the reaction and the specific reaction conditions employed. Suitable leaving groups will be readily apparent to the skilled person and are described in many standard organic chemistry texts, for example: “Advanced Organic Chemistry”, Jerry March. Third Edition, Wiley (1985), page 587, incorporated herein by reference; they include halogen (e.g. Br) and sulfonate esters (e.g. methanesulfonate or trifluoromethanesulfonate).

According to Scheme 3, compounds of Formula (IX) may be prepared from a ketone of Formula (XII) by reaction with a primary amine R³—NH₂ under suitable conditions. The resulting compound of Formula (IX) may then be converted to a compound of Formula (II) by amide formation and deprotection in a manner analogous to that described above in relation to Scheme 1.

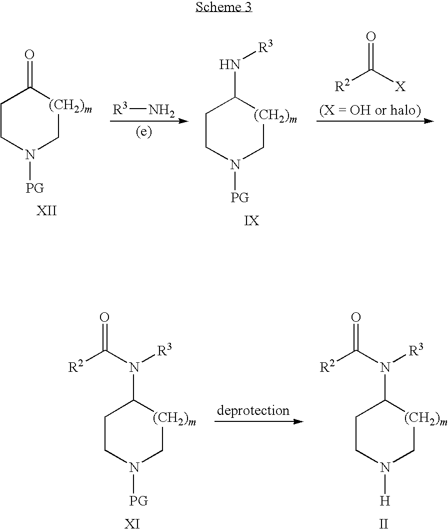

In the above scheme, R², R³ and m are as defined above and PG is a suitable protecting group.

The reaction (e) of the primary amine R³—NH₂ with the ketone (XII) may conveniently be a reductive amination reaction in which the dehydration of the amine and the ketone is followed by reduction of the resultant imine, for example by a metal hydride reagent or hydrogenation, under suitable conditions.

Conveniently, the reaction of the amine and the ketone is carried out in the presence of titanium (IV) tetraisopropoxide in THF at room temperature for 18 hours, followed by reduction by an excess of sodium borohydride in methanol at room temperature for 5 hours.

The skilled person is able to select the most appropriate synthetic route to the desired compound according to Formula (I), (II) or (III). The above schemes may of course be modified as appropriate in accordance with the common general knowledge of those skilled in the art.

For example, the skilled person will of course appreciate that the hydrogen attached to the piperidine or pyrrolidine nitrogen (depending upon the value of m) of the deprotected amide (II) or (V) can be replaced with alternative groups as desired to form a compound of Formula (I) where n is 1 and m is 0 or 1 by the use of conventional synthetic methodologies.

In addition, compounds of Formula (I) where n is 2 and m is 0 can be prepared by analogous processes to those described above using the appropriate starting materials.

It will be appreciated by those skilled in the art that one or more sensitive functional groups may need to be protected and deprotected during the synthesis of a compound of Formula (I), (II) or (III). This may be achieved by conventional techniques, for example as described in “Protective Groups in Organic Synthesis”, 3^{rd} edition, by TW Greene and PGM Wuts. John Wiley and Sons, Inc., 1999, incorporated herein by reference, which also describes methods for the removal of such groups.

It will be apparent to those skilled in the art that certain protected derivatives of compounds of the invention, which may be made prior to a final deprotection stage, may not possess pharmacological activity as such, but may, in certain instances, be administered orally or parenterally and thereafter metabolized in the body to form compounds of the invention which are pharmacologically active. Such derivatives may therefore be described as prodrugs. Further, certain compounds of the invention may act as prodrugs of other compounds of the invention.

According to a further aspect of the invention, there is provided a process for preparing compounds of Formula (I), (II) or (III), which comprises reacting a compound of formula (X):

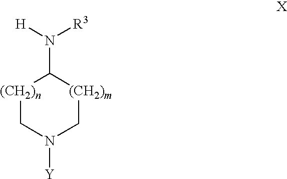

wherein R³, n and m are as defined above and Y is R¹ or a protecting group, with an acid or acyl halide: R²COX, wherein X is OH or halo, and deprotecting if necessary.

Where R³ includes a methylene moiety which is directly bonded to the nitrogen atom, then the compound of Formula (X) may be prepared by reacting a compound of Formula (XXI) with an aldehyde R^{3′}CHO (wherein —CH₂R^{3′} satisfies the definition of R³.).

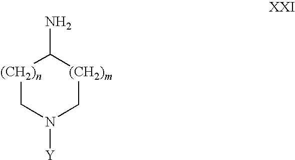

Alternatively, the compound of Formula (X) may be prepared by reacting a compound of Formula (XXI) with a compound R³-L, where L is a leaving group, optionally selected from halide, methanesulfonate and trifluoromethanesulfonate.

Furthermore, the compound of Formula (X) may be prepared by reacting a compound of Formula (XXII) with a compound R³—NH₂.

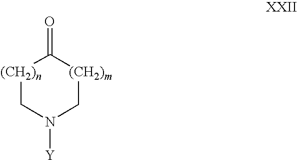

Certain intermediates described above are novel compounds and it is to be understood that all novel intermediates herein form further aspects of the present invention.

Racemic compounds may be separated either using preparative HPLC and a column with a chiral stationary phase, or resolved to yield individual enantiomers utilizing methods known to those skilled in the art. In addition, chiral intermediate compounds may be resolved and used to prepare chiral compounds of the invention.

According to a further aspect of the invention, there is provided one or more metabolites of the compounds of the invention when formed in vivo.

The compounds of the invention may have the advantage that they are more potent, have a longer duration of action, have a broader range of activity, are more stable, have fewer side effects or are more selective, or have other more useful properties than the compounds of the prior art.

The compounds of the invention are useful because they have pharmacological activity in mammals, including humans. Thus, they are useful in the treatment or prevention of disorders in which the regulation of monoamine transporter function is implicated, more particularly disorders in which inhibition of re-uptake of serotonin or noradrenaline is implicated. Furthermore, the compounds of the invention are of use in disorders in which inhibition of both serotonin and noradrenaline is implicated, such as urinary incontinence. Additionally, the compounds of the invention are of use in disorders in which it may be desired to inhibit preferentially the reuptake of one of noradrenaline or serotonin compared with the other, such as pain.

Accordingly the compounds of the invention are useful in the treatment of urinary incontinence, such as genuine stress incontinence (GSI), stress urinary incontinence (SUI) or urinary incontinence in the elderly; overactive bladder (OAB), including idiopathic detrusor instability, detrusor overactivity secondary to neurological diseases (e.g. Parkinson's disease, multiple sclerosis, spinal cord injury and stroke) and detrusor overactivity secondary to bladder outflow obstruction (e.g. benign prostatic hyperplasia (BPH), urethral stricture or stenosis); nocturnal eneuresis; urinary incontinence due to a combination of the above conditions (e.g. stress incontinence associated with overactive bladder); and lower urinary tract symptoms, such as frequency and urgency. The term OAB is intended to encompass both OAB wet and OAB dry.

In view of their aforementioned pharmacological activity the compounds of the invention are also useful in the treatment of depression, such as major depression, recurrent depression, single episode depression, subsyndromal symptomatic depression, depression in cancer patients, depression in Parkinson's patients, postmyocardial infarction depression, paediatric depression, child abuse induced depression, depression in infertile women, post partum depression, premenstrual dysphoria and grumpy old man syndrome.

In view of their aforementioned pharmacological activity the compounds of the invention are also useful in the treatment of cognitive disorders such as dementia, particularly degenerative dementia (including senile dementia, Alzheimer's disease, Pick's disease, Huntingdon's chorea, Parkinson's disease and Creutzfeldt-Jakob disease) and vascular dementia (including multi-infarct dementia), as well as dementia associated with intracranial space occupying lesions, trauma, infections and related conditions (including HIV infection), metabolism, toxins, anoxia and vitamin deficiency; mild cognitive impairment associated with ageing, particularly age associated memory impairment (AAMI), amnestic disorder and age-related cognitive decline (ARCD); psychotic disorders, such as schizophrenia and mania; anxiety disorders, such as generalised anxiety disorder, phobias (e.g. agoraphobia, social phobia and simple phobias), panic disorder, obsessive compulsive disorder, post traumatic stress disorder, mixed anxiety and depression; personality disorders such as avoidant personality disorder and attention deficit hyperactivity disorder (ADHD); sexual dysfunction, such as premature ejaculation, male erectile dysfunction (MED) and female sexual dysfunction (FSD) (e.g. female sexual arousal disorder (FSAD)); premenstrual syndrome; seasonal affective disorder (SAD); eating disorders, such as anorexia nervosa and bulimia nervosa; obesity; appetite suppression; chemical dependencies resulting from addiction to drugs or substances of abuse, such as addictions to nicotine, alcohol, cocaine, heroin, phenobarbital and benzodiazepines; withdrawal syndromes, such as those that may arise from the aforementioed chemical dependencies; cephalic pain, such as migraine, cluster headache, chronic paroxysmal hemicrania, headache associated with vascular disorders, headache associated with chemical dependencies or withdrawal syndromes resulting from chemical dependencies, and tension headache; pain; Parkinson's diseases, such as dementia in Parkinson's disease, neuroleptic-induced Parkinsonism and tardive dyskinesias); endocrine disorders, such as hyperprolactinaemia; vasospasm, such as in the cerebral vasculature; cerebellar ataxia; Tourette's syndrome; trichotillomania; kleptomania; emotional lability; pathological crying; sleep disorder (cataplexy); and shock.

In view of their aforementioned pharmacological activity the compounds of the invention are also useful in the treatment of a number of other conditions or disorders, including hypotension; hot flashes; gastrointestinal tract disorders (involving changes in motility and secretion) such as irritable bowel syndrome (IBS), ileus (e.g. post-operative ileus and ileus during sepsis), gastroparesis (e.g. diabetic gastroparesis), peptic ulcer, gastroesophageal reflux disease (GORD, or its synonym GERD), flatulence and other functional bowel disorders, such as dyspepsia (e.g. non-ulcerative dyspepsia (NUD)) and non-cardiac chest pain (NCCP); and fibromyalgia syndrome.

The compounds of the invention, being serotonin and/or noradrenaline reuptake inhibitors are potentially useful in the treatment of a further range of disorders, including pain.

Physiological pain is an important protective mechanism designed to warn of danger from potentially injurious stimuli from the external environment. The system operates through a specific set of primary sensory neurones and is activated by noxious stimuli via peripheral transducing mechanisms (see Millan, 1999, Prog. NeurobioU 57, 1-164 for a review). These sensory fibres are known as nociceptors and are characteristically small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually in twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g. painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitisation in the periphery, locally around the injury and centrally where the nociceptors terminate. These effects lead to a heightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibres associated with maladaptation and aberrant activity (Woolf & Salter, 2000, Science, 288, 1765-1768).

Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia—Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 1994, Textbook of Pain, 13-44). The activation of nociceptors activates two types of afferent nerve fibres. Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumour related pain (e.g. bone pain, headache, facial pain or visceral pain) or pain associated with cancer therapy (e.g. postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain is currently defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp., 6, S141-S147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability. The exact aetiology of rheumatoid arthritis is unknown, but current hypotheses suggest that both genetic and microbiological factors may be important (Grennan & Jayson, 1994, Textbook of Pain, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder, 2002, Ann Pharmacother., 36, 679-686; McCarthy et al., 1994, Textbook of Pain, 387-395). Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhoea, cystitis and pancreatitis and pelvic pain.

It should be noted that some types of pain have multiple aetiologies and thus can be classified in more than one area, e.g. back pain and cancer pain have both nociceptive and neuropathic components.

Other types of pain include:
pain resulting from musculoskeletal disorders, including myalgia, fibromyalgia, spondylitis, sero-negative (non-rheumatoid) arthropathies, non-articular rheumatism, dystrophinopathy, glycogenosis, polymyositis and pyomyositis;
heart and vascular pain, including pain caused by angina, myocardical infarction, mitral stenosis, pericarditis, Raynaud's phenomenon, scleredoma and skeletal muscle ischemia;
head pain, such as migraine (including migraine with aura and migraine without aura), cluster headache, tension-type headache mixed headache and headache associated with vascular disorders; and
orofacial pain, including dental pain, otic pain, burning mouth syndrome and temporomandibular myofascial pain.

Disorders of particular interest include urinary incontinence, such as mixed incontinence, GSI and USI; pain; depression; anxiety disorders, such as obsessive-compulsive disorder and post traumatic stress disorder; personality disorders, such as ADHD; sexual dysfunction; and chemical dependencies and withdrawal syndromes resulting from chemical dependencies.

Thus, according to further aspects, the invention provides:
i) a compound of the invention for use in human or veterinary medicine;
ii) a compound of the invention for use in the treatment of a disorder in which the regulation of monoamine transporter function is implicated, such as urinary incontinence;
iii) the use of a compound of the invention in the manufacture of a medicament for the treatment of a disorder in which the regulation of monoamine transporter function is implicated;
iv) a compound of the invention for use in the treatment of a disorder in which the regulation of serotonin or noradrenaline is implicated;
v) the use of a compound of the invention in the manufacture of a medicament for the treatment of a disorder in which the regulation of serotonin or noradrenaline is implicated;
vi) a compound of the invention for use in the treatment of a disorder in which the regulation of serotonin and noradrenaline is implicated;
vii) the use of a compound of the invention in the manufacture of a medicament for the treatment of a disorder in which the regulation of serotonin and noradrenaline is implicated;
viii) a compound of the invention for use in the treatment of pain or urinary incontinence, such as GSI or USI;
ix) the use of a compound of the invention in the manufacture of a medicament for the treatment of pain or urinary incontinence, such as GSI or USI;
x) a method of treatment of a disorder in which the regulation of monoamine transporter function is implicated which comprises administering a therapeutically effective amount of a compound of the invention to a patient in need of such treatment;
xi) a method of treatment of a disorder in which the inhibition of the reuptake of serotonin or noradrenaline is implicated which comprises administering a therapeutically effective amount of a compound of the invention to a patient in need of such treatment;
xii) a method of treatment of a disorder in which the inhibition of the reuptake of serotonin and noradrenaline is implicated which comprises administering a therapeutically effective amount of a compound of the invention to a patient in need of such treatment; and
xiii) a method of treating pain or urinary incontinence, such as GSI or USI, which comprises administering a therapeutically effective amount of a compound of the invention to a patient in need of such treatment.

It is to be appreciated that all references herein to treatment include curative, palliative and prophylactic treatment, unless explicitly stated otherwise.

The compounds of the invention may be administered alone or as part of a combination therapy. If a combination of therapeutic agents is administered, then the active ingredients may be administered either sequentially or simultaneously in separate or combined pharmaceutical formulations.

Examples of suitable agents for adjunctive therapy include:
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, phentolamine, terazasin, prazasin or 4-amino-6,7-dimethoxy-2-(5-methane-sulfonamido-1,2,3,4-tetrahydroisoquinol-2-yl)-5-(2-pyridyl) quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT_{2A} receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
an alpha-2-delta ligand such as gabapentin, pregabalin, 3-methylgabapentin, (1α,3α,5α)(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino)ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl) butyl]thio]-6-(trifluoromethyl)-3 pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl),1,4-benzoquinone (CV-6504);
a sodium channel blocker, such as lidocaine;
a 5-HT3 antagonist, such as ondansetron, granisetron, tropisetron, azasetron, dolasetron or alosetron;
an oestrogen agonist or selective oestrogen receptor modulator (e.g. HRT therapies or lasofoxifene);
an alpha-adrenergic receptor agonist, such as phenylpropanolamine or R-450;
a dopamine receptor agonist (e.g. apomorphine, teachings on the use of which as a pharmaceutical may be found in U.S. Pat. No. 5,945,117), including a dopamine D2 receptor agonist (e.g. premiprixal, Pharmacia Upjohn compound number PNU95666; or ropinirole);
a PGE1 agonist (e.g. alprostadil);
   and the pharmaceutically acceptable salts and solvates thereof.

The invention thus provides, in a further aspect, a combination comprising a compound of the invention together with a further therapeutic agent.

For human use the compounds of the invention can be administered alone, but in human therapy will generally be administered in admixture with a suitable pharmaceutical excipient, diluent or carrier selected with regard to the intended route of administration and standard pharmaceutical practice.

For example, the compounds of the invention, can be administered orally, buccally or sublingually in the form of tablets, capsules (including soft gel capsules), ovules, elixirs, solutions or suspensions, which may contain flavouring or colouring agents, for immediate-, delayed-, modified-, sustained-, dual-, controlled-release or pulsatile delivery applications. The compounds of the invention may also be administered via intracavernosal injection. The compounds of the invention may also be administered via fast dispersing or fast dissolving dosage forms.

Such tablets may contain excipients such as microcrystalline cellulose, lactose, sodium citrate, calcium carbonate, dibasic calcium phosphate, glycine, and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention, and their pharmaceutically acceptable salts, may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form. i.e. within the matrix, and/or on the dosage form, i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compounds of the invention can also be administered parenterally, for example, intravenously, intra-arterially, intraperitoneally, intrathecally, intraventricularly, intraurethrally, intrasternally, intracranially, intramuscularly or subcutaneously, or they may be administered by infusion techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

For oral and parenteral administration to human patients, the daily dosage level of the compounds of the invention or salts or solvates thereof will usually be from 10 to 500 mg (in single or divided doses).

Thus, for example, tablets or capsules of the compounds of the invention or salts or solvates thereof may contain from 5 mg to 250 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will also appreciate that, in the treatment of certain conditions (including PE), compounds of the invention may be taken as a single dose on an “as required” basis (i.e. as needed or desired).

### Example Tablet Formulation

In general a tablet formulation could typically contain between about 0.01 mg and 500 mg of a compound according to the present invention (or a salt thereof) whilst tablet fill weights may range from 50 mg to 1000 mg. An example formulation for a 10 mg tablet is illustrated:

| Ingredient | % w/w |
|---|---|
| Free base or salt of compound | 10.000* |
| Lactose | 64.125 |
| Starch | 21.375 |
| Croscarmellose Sodium | 3.000 |
| Magnesium Stearate | 1.500 |

*This quantity is typically adjusted in accordance with drug activity and is based on the weight of the free base.

The compounds of the invention can also be administered intranasally or by inhalation and are conveniently delivered in the form of a dry powder inhaler or an aerosol spray presentation from a pressurised container, pump, spray or nebulizer with the use of a suitable propellant, e.g. dichlorodifluoromethane, trichlorofluoromethane, dichlorotetra-fluoro-ethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount, the pressurised container, pump, spray or nebulizer may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a compound of the invention for delivery to the patient. The overall daily dose with an aerosol will be in the range of from 1 to 50 mg which may be administered in a single dose or, more usually, in divided doses throughout the day.

The compounds of the invention may also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the compounds of the invention can be administered in the form of a suppository or pessary, or they may be applied topically in the form of a gel, hydrogel, lotion, solution, cream, ointment or dusting powder. The compounds of the invention may also be dermally or transdermally administered, for example, by the use of a skin patch. They may also be administered by the ocular, pulmonary or rectal routes.

For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters, wax, cetearyl alcohol, 2-octyldodecanol, benzyl alcohol and water.

The compounds of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

For oral or parenteral administration to human patients the daily dosage levels of compounds of formula (I), and their pharmaceutically acceptable salts, will be from 0.01 to 30 mg/kg (in single or divided doses) and preferably will be in the range 0.01 to 5 mg/kg. Thus tablets will contain 1 mg to 0.4 g of compound for administration singly or two or more at a time, as appropriate. The physician will in any event determine the actual dosage which will be most suitable for any particular patient and it will vary with the age, weight and response of the particular patient. The above dosages are, of course only exemplary of the average case and there may be instances where higher or lower doses are merited, and such are within the scope of the invention.

Oral administration is preferred.

For veterinary use, a compound of the invention is administered as a suitably acceptable formulation in accordance with normal veterinary practice and the veterinary surgeon will determine the dosing regimen and route of administration which will be most appropriate for a particular animal.

Thus according to a further aspect, the invention provides a pharmaceutical formulation containing a compound of the invention and a pharmaceutically acceptable adjuvant, diluent or carrier.

The combinations referred to above may also conveniently be presented for use in the form of a pharmaceutical formulation and thus pharmaceutical formulations comprising a combination as defined above together with a pharmaceutically acceptable adjuvant, diluent or carrier comprise a further aspect of the invention. The individual components of such combinations may be administered either sequentially or simultaneously in separate or combined pharmaceutical formulations.

When a compound of the invention is used in combination with a second therapeutic the dose of each compound may differ from that when the compound is used alone. Appropriate doses will be readily appreciated by those skilled in the art.

The invention is illustrated by the following non-limiting examples in which the following abbreviations and definitions may be used:

APCI Atmospheric pressure chemical ionisation
Arbocel® filter agent

### br Broad

BOC tert-butoxycarbonyl
CDI carbonyldiimidazole
δ chemical shift
d doublet
Δ heat
DCCI dicyclohexylcarbodiimide
DCM dichloromethane

### DMF N,N-dimethylformamide

DMSO dimethylsulfoxide
ES⁺ electrospray ionisation positive scan
ES⁻ electrospray ionisation negative scan
h hours
HOAT 1-hydroxy-7-azabenzotriazole
HOBT 1-hydroxybenzotriazole
HPLC high pressure liquid chromatography
m/z mass spectrum peak
min minutes
MS mass spectrum
NMM N-methyl morpholine
NMR nuclear magnetic resonance
q quartet
s singlet
t triplet
m multiplet
TBTU 2-(1H-benzotriazol-1-yl)-1,1,3,3-tetramethyluronium tetrafluoroborate
Tf trifluoromethanesulfonyl
TFA trifluoroacetic acid
THF tetrahydrofuran
TLC thin layer chromatography
TS⁺ thermospray ionisation positive scan
WSCDI 1-(3-dimethylaminopropyl)-3-ethylcarbodiimide hydrochloride

The Preparations and Examples that follow illustrate the invention but do not limit the invention in any way. All starting materials are available commercially or described in the literature. All temperatures are in ° C. Flash column chromatography was carried out using Merck silica gel 60 (9385). Solid Phase Extraction (SPE) chromatography was carried out using Varian Mega Bond Elut (Si) cartridges (Anachem) under 15 mmHg vacuum. Thin layer chromatography (TLC) was carried out on Merck silica gel 60 plates (5729). Melting points were determined using a Gallenkamp MPO350 apparatus and are uncorrected. NMR was carried out using a Varian-Unity Inova 400 MHz NMR spectrometer or a Varian Mercury 400 MHz NMR spectrometer. Mass spectroscopy was carried out using a Finnigan Navigator single quadrupole electrospray mass spectrometer or a Finnigan aQa APCI mass spectrometer.

Where it is stated that compounds were prepared in the manner described for an earlier Preparation or Example, the skilled person will appreciate that reaction times, number of equivalents of reagents and reaction temperatures may be modified for each specific reaction, and that it may nevertheless be necessary or desirable to employ different work-up or purification conditions.

### Preparation 1: tert-Butyl (3S)-3-(acetylamino)pyrrolidine-1-carboxylate

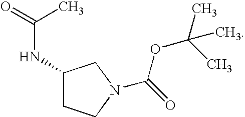

A mixture of tert-butyl (3S)-3-aminopyrrolidine-1-carboxylate (13 g, 70 mmol), acetic anhydride (8.55 g, 84 mmol), and N-methyl morpholine (14.09 g, 140 mmol), in toluene (100 ml), was stirred at room temperature for 72 hours. The reaction mixture was then concentrated under reduced pressure and the residue was partitioned between ethyl acetate and brine. The layers were separated and the aqueous solution was extracted with dichloromethane. The combined organic solutions were dried over sodium sulfate and evaporated under reduced pressure to afford the title compound, 13.89 g.

¹HNMR (CD₃OD, 400 MHz) δ: 1.43 (s, 9H), 1.82 (m, 1H), 1.96 (s, 3H). 2.11 (m, 1H), 3.18 (dd, 1H), 3.38-3.45 (m, 2H), 3.58 (m, 1H), 4.30 (m, 1H); MS APCI+ m/z 229 [MH]⁺.

### Preparation 2: tert-Butyl (3S)-3-(ethylamino)pyrrolidine-1-carboxylate

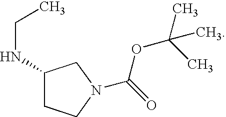

Borane tetrahydrofuran complex (1M in tetrahydrofuran, 18.3 ml, 18.3 mmol) was added dropwise to an ice-cooled solution of the compound described in preparation 1 (1.4 g, 6.13 mmol), in tetrahydrofuran (30 ml), and the reaction mixture was then stirred at 60° C. for 6 hours. The reaction mixture was then quenched by the addition of saturated ammonium chloride solution, and extracted with ethyl acetate. The organic extracts were evaporated under reduced pressure, the resulting residue was re-dissolved in methanol and the solution was heated under reflux for 18 hours. The cooled solution was concentrated under reduced pressure and the resulting residue was purified by column chromatography on silica gel using an elution gradient of dichloromethane:methanol:0.88 ammonia (100:0:0 to 95:5:05) to afford the title compound as a yellow oil, 560 mg, 43%.

¹HNMR (CD₃OD, 400 MHz) δ: 1.30 (t, 3H), 1.44 (s, 9H), 2.02 (m, 1H), 2.36 (m, 1H) 2.99-3.07 (m, 2H), 3.40 (m, 2H), 3.55 (m, 1H), 3.65-3.80 (m, 2H); MS APCI+ m/z 215 [MH]⁺.

### Preparation 3: tert-Butyl(3S)-3-(isobutylamino)pyrrolidine-1-carboxylate

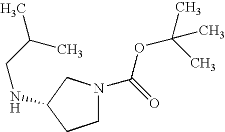

A mixture of tert-butyl (3S)-3-aminopyrrolidine-1-carboxylate (15 g, 80.6 mmol), isobutyraldehyde (7.6 ml, 80.6 mmol), and 10% palladium on charcoal (1.6 g), in ethanol (10 ml), was stirred at room temperature, under 415 kPa (about 60 psi) of hydrogen, for 18 hours. The reaction mixture was then filtered through Arbocel®, and the filtrate was evaporated under reduced pressure to afford the title compound, as a colourless oil, 19.5 g, quantitative.

¹HNMR (CD₃OD, 400 MHz) δ: 0.96 (d, 6H), 1.42 (s, 9H), 1.78 (m, 2H), 2.10 (m, 1H), 2.37-2.45 (m, 2H), 3.04 (m, 1H), 3.30 (m, 2H), 3.42 (m, 1H), 3.58 (m, 1H); MS APCI+ m/z 243 [MH]⁺.

### Preparation 4: tert-Butyl (3S)-3-{[(4-nitrophenyl)sulfonyl]amino}pyrrolidine-1-carboxylate

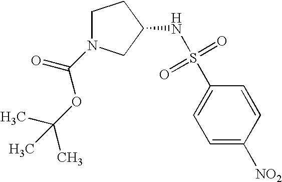

4-Nitrobenzenesulfonyl chloride (10.79 g, 48.7 mmol) was added portionwise to an ice-cooled solution of tert-butyl (3S)-3-aminopyrrolidine-1-carboxylate (10 g, 32.21 mmol), and N-ethyldiisopropylamine (10.3 mmol, 58.86 mmol), in dichloromethane (100 ml), and the reaction mixture was stirred at room temperature for 3.5 hours. Methanol (50 ml) was added and the mixture was washed consecutively with 5% aqueous citric acid solution (200 ml), saturated sodium bicarbonate solution (250 ml) and brine (300 ml). The organic solution, was dried over magnesium sulfate and evaporated under reduced pressure to afford the title compound as a pale yellow solid, 17.7 g, 88%. MS APCI+ m/z 372 [MH]⁺.

### Preparation 5: tert-Butyl (3S)-3-[[(4-nitrophenyl)sulfonyl](propyl)amino]pyrrolidine-1-carboxylate

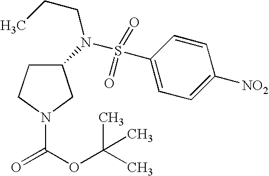

Potassium carbonate (3.8 g, 27.5 mmol) and 1-iodopropane (1.6 ml, 16.44 mmol) were added to a solution of the compound described in preparation 4 (4.6 g, 12.4 mmol) in N,N-dimethylformamide (80 ml), and the reaction mixture was heated at 60° C. for 2 hours, followed by a further 18 hours at room temperature. Additional potassium carbonate (1.7 g, 12.3 mmol) and 1-iodopropane (0.73 ml, 7.44 mmol) were added and the reaction mixture was stirred for a further 2 hours at 60° C. The cooled mixture was concentrated under reduced pressure and the residue was partitioned between water (200 ml) and ethyl acetate (250 ml). The layers separated and the aqueous phase was extracted with further ethyl acetate (200 ml). The combined organic solutions were washed with brine (200 ml), dried over sodium sulfate and evaporated under reduced pressure to afford the title compound as an orange solid, 6.4 g, which was used without further purification.

¹HNMR (CDCl₃, 400 MHz) δ: 0.91 (t, 3H), 1.43 (s, 9H). 1.68 (m, 2H). 1.77-2.00 (m, 2H), 2.97-3.15 (m, 3H), 3.21 (m, 1H), 3.46 (m, 2H), 4.40 (m, 1H), 8.01 (d, 2H), 8.36 (d, 2H); MS APCI+ m/z 414 [MH]⁺.

### Preparation 6: tert-Butyl (3S)-3-(propylamino)pyrrolidine-1-carboxylate

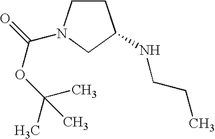

Lithium hydroxide (1.48 g, 61.92 mmol) and mercaptoacetic acid (2.15 ml, 30.96 mmol) were added to a solution of the compound described in preparation 5 (6.4 g, 12.4 mmol) in N,N-dimethylformamide (50 ml). The reaction mixture was stirred at room temperature for 18 hours, followed by 4 hours at 60° C. The mixture was then concentrated under reduced pressure and the residue was partitioned between 5% citric acid solution (200 ml) and ethyl acetate (200 ml). The layers separated, the aqueous, phase was basified using 1M sodium hydroxide solution and then extracted with ethyl acetate (3×250 ml). The combined organic extracts were concentrated under reduced pressure and the residue was partitioned between water/brine (100 ml/10 ml) and dichloromethane (100 ml). The layers were separated, and the organic phase was washed with additional water (100 ml), dried over sodium sulfate and evaporated under reduced pressure to afford the title compound as a pale yellow oil, 2.10 g.

¹HNMR(CDCl₃, 400 MHz) δ: 0.86 (t, 3H), 1.39 (s, 9H). 1.46 (m, 2H), 1.59-1.76 (m, 2H). 1.99 (m, 1H), 2.51 (t, 2H), 2.95-3.07 (m, 1H), 3.19-3.31 (m, 2H), 3.33-3.55 (m, 2H); MS APCI+ m/z 229 [MH]⁺.

### Preparation 7: tert-Butyl (3S)-3-[(cyclobutylcarbonyl)amino]pyrrolidine-1-carboxylate

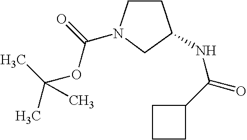

Cyclobutanecarbonylchloride (9 g, 76 mmol) was added to a solution of triethylamine (12.5 ml, 89.7 mmol), and tert-butyl (3S)-3-aminopyrrolidine-1-carboxylate (12.87 g, 69 mmol), in dichloromethane (385 ml), at room temperature, under nitrogen. After stirring for 18 hours at room temperature, the reaction mixture was washed with water, dried over magnesium sulfate and concentrated in vacuo to yield the title product as a light brown glass, (17.4 g, 94%).

¹HNMR (400 MHz, CDCl₃) δ: 1.45 (s, 9H), 1.75-2.00 (m, 3H). 2.07-2.30 (m, 5H), 2.95 (m, 1H), 3.15 (m, 1H), 3.40 (m, 2H), 3.60 (m, 1H), 4.44 (m, 1H), 5.40 (brs, 1H)

### Preparation 8: tert-Butyl (3S)-3-[(cyclobutylmethyl)amino]pyrrolidine-1-carboxylate

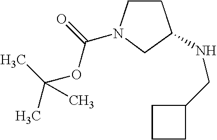

Borane tetrahydrofuran complex (1M in tetrahydrofuran, 100 ml, 100 mmol) was added to a solution of the compound described in preparation 7 (9 g, 33.54 mmol) in anhydrous tetrahydrofuran (100 ml), under nitrogen. The reaction mixture was refluxed for 2 hours, after which time it was cooled to room temperature, quenched with methanol and concentrated in vacuo. The resulting residue was azeotroped with methanol, then re-dissolved in methanol (200 ml), heated under reflux for 18 hours, then concentrated in vacuo. Purification of the residue by chromatography, on silica gel, eluting with dichloromethane:methanol:0.88 ammonia, (95:5:0.5, by volume) afforded the title compound as a gum, (7.67 g, 90%).

¹HNMR (400 MHz, CD₃OD) δ: 1.44 (s, 9H), 1.70 (m, 3H), 1.90 (m, 2H), 2.08 (m, 3H), 2.47 (m, 1H), 2.62 (m, 2H), 3.06 (m, 1H), 3.27 (m, 2H), 3.45 (m, 1H), 3.54 (m, 1H); MS APCI m/z 255 [MH]⁺

### Preparations: tert-Butyl (3S)-3-(sec-butylamino)pyrrolidine-1-carboxylate

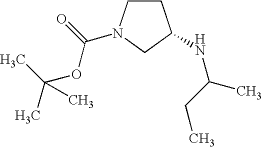

A mixture of tert-butyl (3S)-3-aminopyrrolidine-1-cartjoxylate (4.9 g, 26.3 mmol), 2-butanone (2.6 ml, 28.9 mmol), and 10% palladium on charcoal (600 mg), in ethanol (100 ml), was stirred at room temperature under about 415 kPa (about 60 psi) of hydrogen gas, for 18 hours. The reaction mixture was filtered through Arbocel®, and the filtrate was evaporated under reduced pressure to afford the title compound as a yellow/green oil, 5.70 g, 89%.

¹HNMR (400MHz, CDCl₃) δ: 0.89 (t, 3H), 1.05 (t, 3H), 1.34 (m, 2H), 1.45 (s, 9H), 1.65 (m, 1H), 2.06 (m, 1H), 2.60 (m, 1H), 3.00 (m, 1H), 3.30 (m, 1H), 3.34-3.67 (m, 4H); MS APCI m/z 243 [MH]⁺, 143 [MH-Boc]⁺

### Preparation 10:1-methylcyclobutanecarboxylic acid

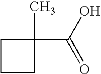

n-butyllithium (2.5M in hexane, 48 ml, 120 mmol) was added dropwise to an ice-cooled solution of N-isopropylpropan-2-amine (17 ml, 120 mmol), in dry tetrahydrofuran (80 ml), under nitrogen. After stirring for 5 minutes, cyclobutanecarboxylic acid (5.3 ml, 50 mmol) was added at 0° C. The reaction mixture was then allowed to warm to room temperature over 30 minutes. Iodomethane (8.52 g, 60 mmol) was added dropwise, and the resulting mixture was stirred for 3 hours at room temperature, under nitrogen. The reaction mixture was then quenched with aqueous hydrochloric acid (40 ml) and partitioned with ether. The organic phase was washed with water and dried over magnesium sulfate (dried). The mixture was then filtered and the solvent was removed by evaporation under reduced pressure to produce an brown oil (3.95 g).

¹HNMR(CDCl₃, 400 MHz) δ: 1.43 (s, 3H), 1.90 (m, 4H), 2.53 (m, 2H)

### Preparation 11: tert-butyl (3S)-3-{[(1-methylcyclobutyl)carbonyl]amino}pyrrolidine-1-carboxylate

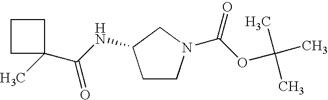

Triethylamine (11.0 ml, 78.6 mmol) was added to a solution of tert-butyl (3S)-3-aminopyrrolidine-1-carboxylate (5.86 g, 31.4 mmol) and the acid described in preparation 10 (3.95 g, 34.6 mmol), in dichloromethane (100 ml), under nitrogen. 2,4,6-tripropyl-1,3,5,2,4,6-trioxatriphosphinane 2,4,6-trioxide (50% solution in ethyl acetate by weight, 21 ml, 34.6 mmol) was added dropwise, at room temperature, under nitrogen, and the reaction mixture was stirred for 3 hours. It was then diluted with 1.5M aqueous potassium carbonate and stirred for 18 hours. After which time the phases were allowed to settle and the organic phase was washed with brine, then dried over magnesium sulfate (dried). The mixture was filtered and the solvent was removed by evaporation under reduced pressure to produce a brown oil (8.3 g).

¹HNMR(CDCl₃, 400 MHz, Rotamers) δ: 1.38 (s, 3H), 1.45 (s, 9H), 1.79 (m, 3H), 1.95 (m, 1H), 2.13 (m, 1H), 2.35 (m, 2H), 2.70 (m, 0.5H). 3.11 (m, 1.5H), 3.41 (m, 2H), 3.62 (m, 1H), 4.43 (m, 1H), 5.44 (m, 1H); MS APCI⁺ m/z 283 [MH]⁺

### Preparation 12: tert-butyl (3S)-3-{[(1-methylcyclobutyl)methyl]amino}pyrrolidine-1-carboxylate

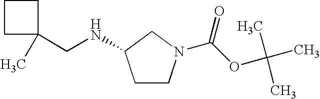

Borane (1M in tetrahydrofuran, 88 ml, 88 mmol) was added to a solution of the compound described in preparation 11 (8.3 g, 29.2 mmol), in anhydrous tetrahydrofuran (88 ml), under nitrogen. The reaction mixture was stirred at reflux for 3 hours, cooled to room temperature and quenched with methanol (50 ml). The solvent was removed by evaporation under reduced pressure and the resulting residue was re-dissolved in methanol (180 ml). After heating the solution at reflux for 18 hours under nitrogen, the solvent was removed by evaporation under reduced pressure, to produce an oil. Purification of the residue by column chromatography on silica gel, eluting with a solvent gradient of cyclohexane:ethyl acetate:pentane (90:10:0.5, by volume) changing to cyclohexane:ethyl acetate:pentane (60:40:0.5, by volume) to produce the title compound (5.35 g) as a colourless oil.

¹HNMR (CDCl₃, 400 MHz) δ: 1.09 (s, 3H), 1.43 (s, 9H), 1.64 (m, 3H), 1.73-1.93 (m, 4H), 2.01 (m, 1H), 2.51 (m, 2H), 3.05 (m, 1H), 3.27 (m, 2H), 3.38-3.63 (m, 2H); MS APCI⁺ m/z 269 [MH]⁺

### Preparation 13: tert-butyl (3S)-3-(cyclopropylmethylamino)pyrrolidine-1-carboxylate

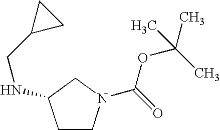

tert-Butyl (3S)-3-(cyclopropylmethylamino)pyrrolidine-1-carboxylate was prepared by a method similar to that described for preparation 29 using tert-butyl (3S)-3-aminopyrrolidine-1-carboxylate, and cyclopropane carboxaldehyde, except that the crude product was purified by chromatography on silica gel eluting with a solvent gradient of dichloromethane changing to dichloromethane:methanol:0.88 ammonia (90:10:1 by volume) to yield the title compound, 5.2 g (81%).

¹HNMR(CDCl₃, 400 MHz) δ: 0.15 (m, 2H), 0.48 (m, 2H), 0.97 (m, 1H), 1.43 (s, 9H), 1.75 (m, 1H), 2.05 (m, 1H), 2.50 (d, 2H), 3.10 (m, 1H), 3.47 (m, 2H), 3.50 (m, 2H); MS APCI⁺ m/z 241 [MH]⁺

### Preparation 14: tert-butyl (3S)-3-(tetrahydro-2H-pyran-4-ylamino)pyrrolidine-1-carboxylate

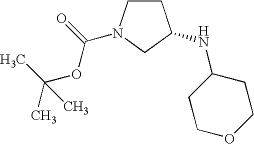

7.5 g (40 mmol, 1 eq.) of tert-butyl (3S)-3-aminopyrrolidine-1-carboxylate and 300 mg of 10% Pd/C were added to a solution of 4.47 g (44.7 mmol, 1.1 eq.) tetrahydro-4H-pyran-4-one, in 250 ml of ethanol, and the reaction mixture was left under 415 kPa (about 60 psi) of hydrogen gas for 18 hours. It was then filtered through Arbocel®, washing through thoroughly with ethyl acetate. The filtrate was concentrated in vacuo and the crude product was purified by column chromatography on silica gel to yield the desired product, 6.7 g (61%).

¹HNMR (CDCl₃, 400 MHz) □: 1.39 (m, 2H), 1.46 (s, 9H), 1.67 (m, 1H), 1.82 (m, 2H), 2.07 (m, 1H), 2.72 (m, 1H), 3.02 (brm, 1H), 3.31-3.39 (m, 5H), 3.59 (brm, 1H), 3.96 (m, 2H); MS ES+ m/z 271 [MH]⁺

### Preparation 15: tert-butyl 3-(cyclopropylamino)pyrrolidine-1-carboxylate

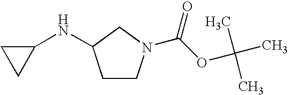

Acetic acid (5 drops) was added to a solution of tert-butyl 3-oxopyrrolidine-1-carboxylate (200 mg, 1.1 mmol), and cyclopropanamine (83 μl, 1.2 mmol), in dichloromethane (5 ml), under nitrogen. Sodium triacetoxyborohydride (504 mgs, 2.38 mmol) was added portionwise and the reaction mixture was stirred at room temperature for 3 hours. After this time the resultant mixture was quenched with 1.5M aqueous potassium carbonate (8 ml) and the layers were separated. The organic phase was dried over magnesium sulfate (dried), filtered and the solvent was removed by evaporation under reduced pressure to produce the title compound (256 mg) as brown oil

¹HNMR (CDCl₃, 400 MHz) δ: 0.36 (m, 2H), 0.48 (m, 2H), 1.45 (s, 9H), 1.89 (m, 2H), 2.05 (m, 1H), 2.13 (m, 1H), 3.12 (m, 1H), 3.27-3.67 (m, 4H); MS APCI⁺ m/z 227 [MH]⁺

### Preparation 16: tert-butyl (3S)-3-{[(1-methylcyclopropyl)carbonyl]amino}pyrrolidine-1-carboxylate

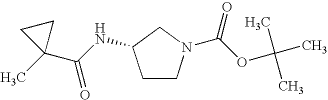

The title compound was prepared by a similar method to that described for preparation 11 using 1-methylcyclopropanecarboxylic acid, to produce the desired product, 6.8 g (95%) as a white solid.

¹HNMR (CDCl₃, 400 MHz) δ: 0.57 (m, 2H), 1.18 (m, 2H), 1.29 (s, 3H), 1.45 (s, 9H), 1.80 (s, 1H), 2.14 (m, 1H), 3.16 (m, 1H), 3.42 (m, 2H), 3.62 (m, 1H), 4.45 (m, 1H), 5.74 (m, 1H); MS APCI⁺ m/z 269 [MH]⁺

### Preparation 17: tert-butyl (3S)-3-{[(1-methylcyclopropyl)methyl]amino}pyrrolidine-1-carboxylate

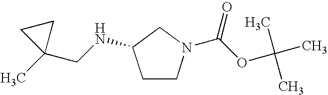

The title compound was prepared by a similar method to that described for preparation 12 using the amide described in preparation 16, except that during chromatography a solvent gradient of ethyl acetate:pentane (20:80, by volume) changing to ethyl acetate:pentane (30:70, by volume) was used to produce the desired product, 3.9 g (60%) as a white solid.

¹HNMR (CD₃OD, 400 MHz) δ: 0.53 (m, 2H), 0.63 (m, 2H), 1.22 (s, 3H), 1.45 (s, 9H), 2.08 (m, 1H), 2.36 (m, 1H), 2.94 (q, 2H), 3.39 (m, 2H), 3.56 (m, 1H), 3.80 (m, 2H); MS APCI⁺ m/z 255 [MH]⁺

### Preparation 18: tert-butyl (3S)-3-({[1-(trifluoromethyl)cyclopropyl]carbonyl}amino)pyrrolidine-1-carboxylate

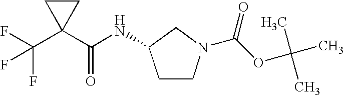

The title compound was prepared by a similar method to that described for preparation 11 using tert-butyl (3S)-3-aminopyrrolidine-1-carboxylate and 1-(trifluoromethyl)cyclopropane carboxylic acid to produce the desired product, 5.7 g (86%) as a brown solid.

¹HNMR (CD₃OD, 400 MHz) δ: 1.25 (m, 3H), 1.45 (s, 9H), 1.90 (m, 1H), 2.12 (m, 1H), 3.18 (m, 1H), 3.36-3.64 (m, 4H), 4.35 (m, 1H); MS APCI⁺ m/z 323 [MH]⁺

### Preparation 19: tert-butyl (3S)-3-({[1-(trifluoromethyl)cyclopropyl]methyl}amino)pyrrolidine-1-carboxylate

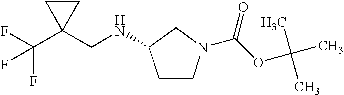

The title compound was prepared by a similar method to that described for preparation 12 using the amide described in preparation 18, except that saturated aqueous ammonium chloride (50 ml), and methanol (50 ml), at reflux, under nitrogen, was used to hydrolyse the borane complexes to produce the desired product, 2.7 g (49%) as a foam.

¹HNMR (CD₃OD, 400 MHz) δ: 0.79 (s, 2H), 0.94 (s, 2H), 1.24 (m, 1H), 1.45 (s, 9H), 1.71 (m, 1H), 2.04 (m, 1H), 2.82 (q, 2H), 3.07 (m, 1H), 3.28 (m, 1H), 3.45 (m, 2H); MS APCI⁺ m/z 309 [MH]⁺

### Preparation 20: tert-butyl (3S)-3-{(cyclobutylmethyl)[2-(methylthio)benzoyl]amino}pyrrolidine-1-carboxylate

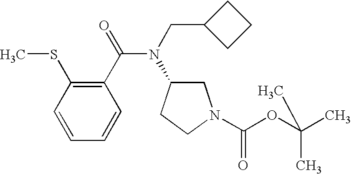

0.250 g (0.98 mmol, 1 eq) of tert-butyl (3S)-3-[(cyclobutylmethyl)amino]pyrrolidine-1-carboxylate (preparation 8) was dissolved in 5 ml of toluene. 0.24 g (1.3 mmol, 1.3 eq.) of 2-(methylthio)benzoyl chloride were then added, followed by 0.27 ml (1.96 mmol, 2 eq.) of triethylamine. The solution was stirred at room temperature for 18 hours, then filtered through a hydrophobic membrane. The product was purified by chromatography on silica gel using a gradient of pentane changing to pentane:ethyl:acetate (50:50 by volume), affording 0.30 g (76%) of the title compound as an oil.

¹HNMR (400 MHz. CDCl₃) δ: 1.43 (broad s, 9H), 1.7-2.2 (m, 7H), 2.2-2.4 (m, 1H), 2.44 (s, 3H), 2.6-2.9 (m, 1H), 2.9-3.85 (m, 6H), 3.85-4.15, 4.45-4.7 (multiplets, 1H) 7.1-7.3 (m, 2H), 7.3-7.4 (m, 2H); MS APCI+ m/z 405 [MH]⁺, 349-[MH-isobutylene]⁺, 305[MH-BOC]⁺

### Preparation 21: tert-butyl (3S)-3-[sec-butyl(2-phenoxybenzoyl)amino]pyrrolidine-1-carboxylate

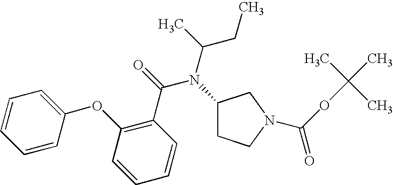

The title compound was prepared by a similar method to that described for preparation 43, using the amine described for preparation 9, and 2-phenoxybenzoyl chloride (Tetrahedron (1988), 44(18), 5857-60) to produce the desired product, 420 mg (84%) as a colourless oil.

MS APCI⁺ m/z 439 [MH]⁺

### Preparation 22: tert-butyl (3S)-3-anilinopyrrolidine-1-carboxylate

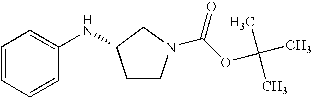

1,1′-Binaphthalene-2,2′-diylbis(diphenylphosphine) (1.34 g, 2.2 mmol) was added to a solution of tert-butyl (3S)-3-aminopyrrolidine-1-carboxylate (4 g, 21.5 mmol) in toluene (86 ml), at room temperature, under nitrogen. To the reaction mixture was added sodium t-butoxide (1.9 g, 25.8 mmol) and tris(dibenzylideneacetone)dipalladium(0) (984 mg, 1.1 mmol). After stirring the reaction at 100° C. for 24 hours, the solvent was removed by evaporation under reduced pressure. The resulting residue was dissolved in minimum dichloromethane and purified by chromatography on silica gel eluting with dichloromethane:methanol:ammonia (99:1:0.1, by volume) to produce impure product. The purification process by chromatography was repeated using a solvent gradient of ethyl acetate:pentane (0:100, by volume) changing to ethyl acetate:pentane (20:80, by volume) to produce the title compound (3.2 g) as light brown solid.

¹HNMR (CDCl₃, 400 MHz) δ: 1.45 (s, 9H), 1.88 (m, 1H), 2.18 (m, 1H), 3.25 (m, 1H), 3.46 (m, 2H), 3.63-4.08 (m, 3H), 6.61 (d, 2H), 6.73 (t, 1H), 7.18 (t, 2H); MS APCI⁺ m/z 263 [MH]⁺

### Preparation 23: tert-butyl (3S)-3-(isopropylamino)pyrrolidine-1-carboxylate

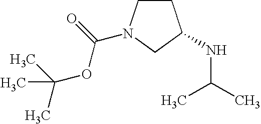

8.5 g (45.6 mmol, 1 eq) of tert-butyl (3S)-3-aminopyrrolidine-1-carboxylate, 30 ml of acetone and 150 ml of ethanol were mixed together. 2 g of 10% palladium on carbon were then added and the reaction mixture was hydrogenated under 415 kPa (about 60 psi) of hydrogen, at room temperature, for 18 hours. The mixture was then filtered through Celite® under a nitrogen atmosphere, and the solvent was removed under reduced pressure. 50 ml of toluene were added and the solvent was removed under reduced pressure, affording 10.4 g (100%) of the title product.

¹HNMR (CDCl₃, 400 MHz) δ: 1.05 (m, 6H), 1.42 (s, 9H), 1.65-1.90 (b, 2H), 2.10 (m, 1H), 1.80-3.1 (m, 2H) 3.15-3.80 (m, 4H); LCMS ELSD-APCI⁺ m/z 229 [MH]⁺.

### Preparation 24: Tert-butyl (3S)-3-{cyclopentyl[2-(methylthio)benzoyl]amino}pyrrolidine-1-carboxylate

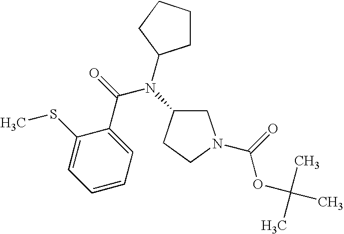

To a solution of 2-(methylthio)benzoic acid (192 mg, 1.14 mmol) in dichloromethane (3 ml) was added oxalyl chloride (0.25 ml, 2.85 mmol), and dimethylformamide (0.005 ml). The reaction mixture was stirred at room temperature for 0.75 hours and then the solvent was removed in vacuo. The acid chloride was redissolved in toluene and added dropwise to a solution of tert-butyl (3S)-3-(cyclopentylamino)pyrrolidine-1-carboxylate (223 mg, 0.876 mmol) (preparation 29) and triethylamine (0.25 ml, 1.75 mmol), in toluene (3 ml). The reaction mixture was stirred at room temperature for 18 hours then, heated at 65° C. for 4 hours. The solution was then filtered through a hydrophobic cartridge and the solvent was removed in vacuo. The crude material was purified by column chromatography using an ISCO® silica cartridge eluting with a gradient of 0-100% ethyl acetate:pentane. The title compound was obtained as a colourless gum (289 mg, 0.71 mmol, 81%).

¹HNMR (400 MHz. CDCl₃₎: δ 0.83-1.40 (m, 6H), 1.45 (s, 9H), 1.50-2.15 (m, 5H), 2.46 (s, 3H), 2.86-4.07 (m, 5H), 7.09 (d, 1H), 7.18 (m, 1H), 7.31 (m, 2H); LCMS m/z ELSD-APCI⁺ 405 [MH]⁺.

### Preparation 25: tert-butyl (3S)-3-[(cyclobutylmethyl)(2-phenoxybenzoyl)amino]pyrrolidine-1-carboxylate

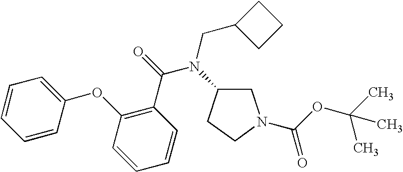

0.30 g (1.18 mmol, 1 eq) of tert-butyl (3S)-3-[(cyclobutylmethyl)amino]pyrrolidine-1-carboxylate (preparation 8) was dissolved in 5 ml of methylene chloride. Then 0.35 g (1.5 mmol, 1.27 eq.) of 2-phenoxybenzoyl chloride (Tetrahedron (1988), 44(18), 5857-60) and 0.33 mL (2.36 mmol, 2 eq.) of triethylamine were added. After work-up, the solvent was removed under reduced pressure and the residue was purified by chromatography on silica gel, using a gradient of pentane:ethyl acetate (4:1 by volume) changing to pentane:ethyl acetate (1:1 by volume), affording 0.411 g (77%) of the title compound as a gum.

¹HNMR (400 MHz, CDCl₃) δ: 1.46 (m, 9H), 1.76 (m, 3H), 1.93 (m, 4H), 2.30-2.50, 2.58 (multiplets, 1H), 3.04-3.36 (m, 4H), 3.36-3.70 (m, 3H), 4.28, 4.35-4.55 (multiplets, 1H), 6.95 (m, 3H), 7.10 (m, 2H), 7.30 (m, 4H); LCMS ELSD-APCI⁺ m/z 395 [MH-isobutylene]⁺, 351 [MH-BOC]⁺.

### Preparation 26: Tert-butyl (3S)-3-[(2,2-dimethylpropyl)amino]pyrrolidine-1-carboxylate

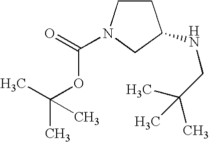

To a solution of tert-butyl (3S)-3-aminopyrrolidine-1-carboxylate (5 g, 26.8 mmol), in ethanol (100 ml), was added trimethylacetaldehyde (2.54 g, 29.5 mmol) and 10% palladium on carbon (600 mg). The reaction mixture was stirred at room temperature under 415 kPa (about 60 psi) of hydrogen, for 18 hours. It was then filtered through Arbocel® and the solvent was evaporated in vacuo. The crude material was purified by column chromatography over silica gel eluting with dichloromethane:methanol:0.880 ammonia (98:2: 0.2-95:5:0.5). The title compound was obtained as a red/brown oil (6.56 g, 25.5 mmol, 95%).

¹HNMR (CDCl₃, 400 MHz) δ: 0.90 (s, 9H), 1.46 (s, 9H), 1.70 (m, 1H), 2.02 (m, 1H), 2.35 (t, 2H), 3.07 (m, 1H), 3.29 (m, 2H), 3.47 (m, 2H); LRMS m/z ELSD-APCI⁺ 257 [MH]⁺.

### Preparation 27: Tert-butyl (3S)-3-(formylamino)pyrrolidine-1-carboxylate

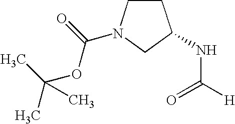

To a solution of pentafluorophenol (5.92 g, 32.2 mmol) in diethyl ether (54 ml), stirring at 0° C., was added formic acid (1.78 g, 38.7 mmol), and N,N′-dicyclohexylcarbodiimide (6.64 g, 32.2 mmol). The reaction mixture was stirred at 0° C. for 2 hours. The resultant precipitate was removed by filtration and the filtrate was concentrated in vacuo. The residue was redissolved in dichloromethane and added dropwise to a solution of tert-butyl (3S)-3-aminopyrrolidine-1-carboxylate (3 g, 16.1 mmol) in dichloromethane (80 ml). The reaction mixture was stirred at room temperature for 18 hours, after which time the solvent was removed in vacuo. The crude product was purified by column chromatography using an ISCO® silica cartridge eluting with a gradient of 0-10% dichloromethane:methanol. The title compound was isolated as a brown gum (2.29 g, 10.7 mmol, 66%).

¹HNMR (CDCl₃, 400 MHz) δ: 1.45 (s, 9H), 1.62-1.99 (m, 1H), 2.16 (m, 1H), 3.12-3.33 (m, 1H), 3.33-3.54 (m, 2H), 3.61 (q, 1H), 4.53 (m, 1H), 5.80-6.05 (m, 1H), 8.15 (s, 1H); LRMS m/z ELSD-APCI⁺ 215 [MH]⁺.

### Preparation 28: Tert-butyl (3S)-3-(methylamino)pyrrolidine-1-carboxylate

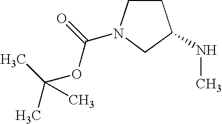

To a solution of tert-butyl (3S)-3-(formylamino)pyrrolidine-1-carboxylate (2.29 g, 10.7 mmol) (from preparation 27), in tetrahydrofuran (35 ml), stirring at 0° C., was added a 1M solution of borane, in tetrahydrofuran (32 ml, 32 mmol). The reaction mixture was stirred at room temperature for 18 hours and then it was quenched with methanol (10 ml), and concentrated in vacuo. The resultant white solid was redissolved in methanol (30 ml) and heated to reflux for 18 hours. The solvent was evaporated in vacuo to yield the title compound as a pale yellow oil (2.45 g, 12.2 mmol, 100%).

¹HNMR (400 MHz, CDCl₃): δ 1.45 (s, 9H), 1.47-1.77 (m, 2H), 1.71 (m, 1H), 2.03 (m, 1H), 2.43 (s, 3H), 3.04-3.26 (m, 1H), 3.29-3.58 (m, 2H); LRMS m/z ELSD-APCI⁺ 201 [MH]⁺.

### Preparation 29: tert-butyl (3S)-3-(cyclopentylamino)pyrrolidine-1-carboxylate

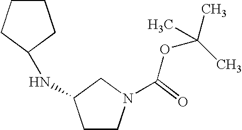

Cyclopentanone (12.7 ml, 143 mmol) was added to tert-butyl (3S)-3-aminopyrrolidine-1-carboxylate (26.6 g, 143 mmd) in a mixture of methanol:toluene 3:1 (600 ml:200 ml), and the reaction mixture was stirred at room temperature for 1.5 hours, under nitrogen. The reaction mixture was then evaporated to 50 ml, azeotroped three times with methanol:toluene 3:1 (600 ml:200 ml) and concentrated in vacuo. It was then taken up in methanol (250 ml), cooled down to 0° C., and sodium borohydride (7.5 g, 200.2 mmol) was added portionwise. After completion of the reaction, water (50 ml) was added and the solvent was evaporated. The residue was diluted with more water (150 ml) and extracted three times with dichloromethane (250 ml). The organic phases were combined, dried over magnesium sulfate, and concentrated in vacuo to provide the title compound as a gum, 36.1 g (99.4%).

¹HNMR (CDCl₃, 400 MHz) δ: 1.18 (brs, 1H), 1.28 (m, 2H), 1.44 (s, 9H), 1.52 (m, 2H), 1.67 (m, 3H), 1.83 (m, 2H), 2.05 (m, 1H), 2.98 (m, 1H), 3.08 (m, 1H), 3.30 (m, 2H), 3.45 (m, 1H), 3.58 (m, 1H); MS APCI⁺ m/z 255[MH]⁺

### Preparation 30: tert-Butyl (3S)-3-(cyclohexylamino)pyrrolidine-1-carboxylate

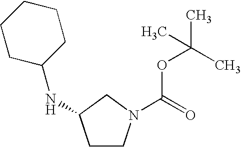

tert-Butyl (3S)-3-(cyclohexylamino)pyrrolidine-1carboxylate was prepared by a method similar to that described for preparation 29 using tert-butyl (3S)-3-aminopyrrolidine-1-carboxylate and cyclohexanone, to yield the desired product, 5.9 g (82%).

¹HNMR (CDCl₃, 400 MHz) δ: 1.09 (m, 2H), 1.24 (m, 3H), 1.45 (s, 9H), 1.62 (m, 2H), 1.72 (m, 2H), 1.88 (m, 2H), 2.06 (m, 1H), 2.48 (m, 1H), 3.01 (m, 1H), 3.30 (m, 1H), 3.45 (m, 2H), 3.55-3.62 (m, 1H); MS APCI⁺ m/z 269 [MH]⁺

### Preparation 31: tert-butyl (3S)-3-(cyclobutylamino)pyrrolidine-1-carboxylate

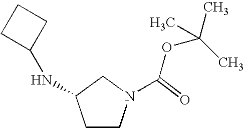

tert-Butyl (3S)-3-(cyclobutylamino)pyrrolidine-1-carboxylate was prepared by a method similar to that described for preparation 29 using tert-butyl (3S)-3-aminopyrrolidine-1-carboxylate and cyclobutanone (15 equivalents: 10 eq. added first, 5 eq. added before the second azeotropic removal of water) except that the crude product was purified by column chromatography on silica gel to yield the title compound, 542 mg (28%).

¹HNMR(CD₃OD, 400 MHz) δ: 1.45 (s, 9H), 1.70 (m, 3H), 1.81 (m, 3H), 2.05 (m, 1H), 2.21 (m, 2H), 3.03 (m, 1H), 3.26 (m, 2H), 3.47 (m, 2H); MS APCI⁺ m/z 241 [MH]⁺

### Preparation 32: 5-Fluoro-2-phenoxybenzonitrile

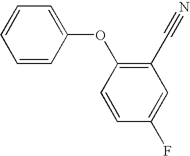

Potassium carbonate (2.90 g, 21 mmol) was added to a solution of phenol (791 mg, 8.4 mmol) in 30 ml of N,N-dimethylformamide. The reaction mixture was stirred for 15 minutes at room temperature, and then 2,5-difluorobenzonitrile (973 mg, 7 mmol) was added. The resulting mixture was heated to 100° C. for 18 hours, after which time it was allowed to cool to room temperature. It was then concentrated in vacuo. The resulting residue was diluted with ethyl acetate (50 ml), and then washed with water (2×100 ml) and 1M sodium hydroxide solution (100 ml). The organic layer was dried over sodium sulfate, filtered and evaporated under reduced pressure to afford the title compound, as a yellow oil, 1.449 g, 97%.

¹HNMR (400 MHz, CDCl₃) δ: 6.87 (q, 1H), 7.05-(d, 2H), 7.21 (m, 2H), 7.33-7.43 (m, 3H); LRMS APCI+ m/z 214 [MH]⁺

### Preparation 33: 5-Fluoro-2-phenoxybenzoic acid

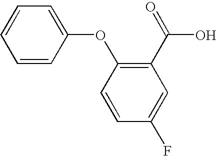

A 6M sodium hydroxide solution (16 ml) was added to the compound described for preparation 32 (1.45 g, 6.80 mmol) in ethanol (16 ml). The resulting mixture was refluxed for 18 hours, allowed to cool to room temperature then cooled on ice and slowly acidified with a 6M aqueous hydrochloric acid solution. The white precipitate was collected by filtration, washed with water and dried under vacuum to afford the title compound, 1.46 g, 93%.

¹HNMR (400 MHz, CD₃OD) δ: 6.90 (d, 2H), 7.04 (m, 2H), 7.31 (m, 3H), 7.60 (dd, 1H); MS APCI— m/z 231 [M-H]⁻

### Preparation 34: 2-(ethylthio)benzoic acid

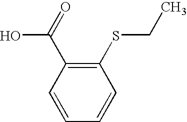

A 1M sodium hydroxide solution (10 ml) was added to 2-mercaptobenzoic acid (1 g, 6.4 mmol) in ethanol (10 ml), followed by iodoethane (1 g, 6.4 mmol). The reaction mixture was stirred for 72 hours and then the ethanol was evaporated under reduced pressure. The reaction mixture was cooled in an ice bath and acidified to pH 1 with 2N aqueous hydrochloric acid. The precipitate was collected by filtration, washed with water and dried under reduced pressure to afford the title compound, 1.09 g, 98%.

¹HNMR (400 MHz, CD₃OD) δ: 1.32 (t, 3H), 2.9 (q, 2H), 7.13 (t, 1H), 7.38 (d, 1H), 7.43 (t, 1H), 7.88 (d, 1H); MS APCI- m/z 181 [M-H]⁻

### Preparation 35: 1-bromo-2-(1,1-difluoroethyl)benzene

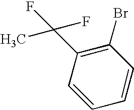

To a mixture of bis(2-methoxyethyl)aminosulfur trifluoride (Deoxo Fluor) (16.0 g, 72.40 mmol, 1.5 eq.) and four drops of methanol was added, dropwise, 2-bromo acetophenone (9.60 g, 48.26 mmol, 1 eq.). Upon completion of the addition, the reaction mixture was placed under a nitrogen atmosphere and heated to 70° C. for 18 hours. The solution was then added dropwise to 100 ml of water, and the resulting mixture was extracted twice with 150 ml of ether. The combined ether phases were washed twice with water, twice with aqueous sodium bicarbonate, once with 10% aqueous citric acid, once with brine, and then dried over magnesium sulfate. The solvent was removed under reduced pressure, affording 6.70 g (63%) of the title compound as a colourless oil (containing a styrene derivative as an impurity) which was used in the next step without further purification.

¹HNMR (CDCl₃, 400 MHz) [symbol] : 2.05 (t, 3H), 7.25 (m, 1H), 7.35 (m, 1H), 7.60 (m, 2H).

### Preparation 36: 2-(1,1-Difluoro-ethyl)-benzoic acid

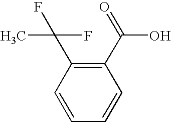

The compound described in preparation 35 (4.00 g, 18.1 mmol, 1 eq) was dissolved in 40 ml of tetrahydrofuran. The solution was cooled to −78° C. and then 2M Butyl lithium in cyclohexane (10.8 ml, 21.7 mmol, 1.2 eq.) was added dropwise. The solution was stirred at −78° C. for 30 minutes and carbon dioxide was bubbled through the solution. The reaction mixture was then stirred for one hour at −78° C., after which time it was allowed to reach room temperature. The solvent was removed under reduced pressure, and then water (20 ml) and 1N sodium hydroxide (20 ml) were added. The solution was then washed twice with hexane (20 ml). The combined hexane phases were re-extracted twice with 1N sodium hydroxide (10 ml). The combined aqueous phases were acidified with 2N aqueous hydrogen chloride and extracted with ether. The ether phase was dried over magnesium sulfate and the solvent was removed under reduced pressure. The resulting residue was purified by chromatography on silica gel using a gradient of methylene chloride, changing to methylene chloride:methanol:ammonia (100:10:1 by volume), affording 1.90 g (56%) of the title compound, which contained small amounts of a styrene derivative and pentanoic acid.

The product (1.0 g, 5.37 mmol, 1 eq.) was dissolved in acetonitrile (30 ml) and the solution was cooled to 0° C. Ruthenium trichloride (0.11 g, 0.54 mmol, 0.1 eq.) and sodium periodate (0.46 g, 2.15 mmol, 0.4 eq.) were added. The solution was then stirred at 0° C. for 3 hours, after which time the solvent was removed under reduced pressure. 2N aqueous hydrogen chloride (15 ml) was added and the mixture was extracted three times with ethyl acetate (50 ml). The combined ethyl acetate phases were washed three times with 2N aqueous hydrogen chloride (3×5 ml) and dried over magnesium sulfate. The solvent was removed under reduced pressure to afford 1.2 g of a dark oil. This oil was purified by chromatography on silica gel using a gradient of methylene chloride, changing to methylene chloride:methanol:ammonia (100:10:1 by volume) affording 400 mg of the title compound.

¹HNMR (CDCl₃, 400 MHz) δ; 2.10 (t, 3H), 7.50 (m, 1H), 7.60 (m, 2H), 7.75 (m, 1H), 11.0 (b, 1H); LCMS ELSD-APCI⁻ m/z 185 [M-H]⁻.

### Preparation 37: 2-(1,1-difluoroethyl)benzoyl chloride

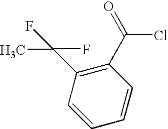

The compound described in preparation 36 (0.44 g, 2.36 mmol, 1 eq) was dissolved in dichloromethane (30 ml), and oxalyl chloride (1.0 g, 7.87 mmol, 3.3 eq.) was added. The solution was stirred at room temperature for one hour, after which time the solvent was removed under reduced pressure affording 0.44 g (92%) of the title compound as an oil.

¹HNMR (CDCl₃, 400 MHz) [symbol] : 2.0 (t, 3H), 7.60 (m, 3H), 7.80 (m, 1H)

### Preparation 38: 2-(2-Cyclopropylphenyl)-4,4-dimethyl-4,5-dihydro-1,3-oxazole

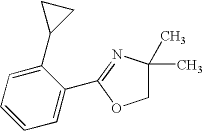

To a cooled (0° C.) solution of 2-(2′-methoxyphenyl)-4,4-dimethyl-2-oxazoline (5.8 g, 28.33 mmol) in tetrahydrofuran (34 ml) was added a 0.5M solution of cyclopropylmagnesium bromide in tetrahydrofuran (170 ml, 85 mmol). The reaction mixture was allowed to warm to room temperature and stirred for 72 hours. It was then quenched with a saturated aqueous solution of ammonium chloride (100 ml), and the tetrahydrofuran was evaporated in vacuo. The residue was extracted with diethyl ether (3×100 ml) and the combined organic extracts were dried over sodium sulfate, filtered and evaporated in vacuo. The crude material was purified by column chromatography using an ISCO® silica cartridge eluting with a gradient of 0-20% ethyl acetate: pentane. The title compound was obtained as a white solid (4.5 g, 20.9 mmol, 74%).

¹HNMR (400 MHz, CDCl₃): δ 0.67 (m, 2H), 0.96 (m, 2H), 1.41 (s, 6H), 2.63 (m, 1H), 4.10 (s, 2H), 6.96 (d, 1H), 7.16 (t, 1H), 7.31 (t, 1H), 7.65 (d, 1H).

### Preparation 39: 2-(2-Cyclopropylphenyl)-3,4,4-trimethyl-4,5-dihydro-1,3-oxazol-3-ium

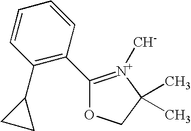

To a solution of the compound described in preparation 38 (3.94 g, 18.3 mmol) in acetone (32 ml), was added iodomethane (9.1 ml, 146.4 mmol), and the reaction mixture was heated to reflux for 18 hours. The solvent was then removed in vacuo and the residue was triturated with ether (3×40 ml). The title compound was obtained as a pale yellow solid (5.9 g, 16.4 mmol, 90%).

¹HNMR (CD₃OD, 400 MHz) δ: 0.81 (m, 2H), 1.12 (m, 2H), 1.73 (s, 6H), 1.94 (m, 1H), 3.31 (m, 3H), 5.05 (s, 2H), 7.31 (d, 1H), 7.47 (t, 1H), 7.68 (m, 2H).

### Preparation 40: 2-Cyclopropylbenzoic acid

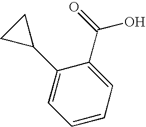

To a solution of compound described in preparation 39 (5.9 g, 16.5 mmol) in methanol (67 ml) was added 20% aqueous sodium hydroxide (70 ml), and the reaction mixture was heated to reflux for 3 hours, then left to stir at room temperature for a further 18 hours. The solution was cooled to 0° C. and acidified to pH 1 with concentrated aqueous hydrogen chloride (30 ml). The methanol was evaporated in vacuo and the residue was extracted with diethyl ether (3×100 ml). The combined organic phases were dried over magnesium sulfate, filtered and evaporated in vacuo. The title compound was obtained as a pale yellow solid (2.65 g, 16.3 mmol, 99%).

¹HNMR (400 MHz, CDCl₃): δ 0.74 (m, 2H), 1.05 (m, 2H), 2.80 (m, 1H), 7.05 (d, 1H), 7.24 (t, 1H), 7.45 (t, 1H), 7.98 (d, 1H); LRMS m/z ELSD-APCI⁻ 161 [MH]⁻.

### Preparation 41: 2-(2-cyclobutylphenyl)-4,4-dimethyl-4,5-dihydro-1,3-oxazole

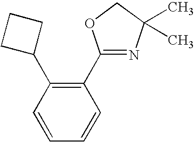

The title compound was prepared as described for preparation 38, by adding cyclobutyl magnesium chloride (55 mmol, 3 eq.) in tetrahydrofuran (50 ml) to a solution of 2-(2-methoxyphenyl)-4,4-dimethyl-4,5-dihydro-1,3-oxazole (3.60 g, 18.4 mmol, 1 eq.) in tetrahydrofuran (50 ml) at 0° C. affording, after work-up, 4.27 g of the crude title compound, which was used directly in the next step.

¹HNMR (400 MHz, CDCl₃) δ: 1.4 (s, 6H), 1.75-1.87 (m, 1H), 1.94-2.07 (m, 1H), 2.07-2.19 (m, 2H), 2.31-2.41 (m, 2H), 4.09 (s, 2H), 4.12-4.2 (m, 1H), 7.17-7.23 (t of m, 1H), 7.34-7.43 (m, 2H), 7.64 (d, 1H); MS APCI+ 230 [MH]⁺.

### Preparation 42: 2-cyclobutylbenzoic acid

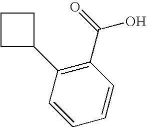

The crude compound described in preparation 41 (4.13 g) was dissolved in a mixture of dioxane (30 ml) and aqueous 6N hydrogen chloride (30 ml). The solution was refluxed until completion of the reaction. The solvent was then removed under reduced pressure and the mixture was extracted twice with ether (70 ml). The combined ether phases were dried over magnesium sulfate and concentrated under reduced pressure, affording the title compound as a brown solid (3.06 g, 97%).

¹HNMR/CD3OD, 400 MHz) δ: 1.75-1.85 (m, 1H), 1.95-2.17 (m, 3H), 2.35-2.43 (m, 2H), 4.2 (quintet, 1H), 7.23 (t, 1H), 7.4-7.5 (m, 2H), 7.72 (m, 1H); MS APCI⁻ m/z 175 [M-H]⁻.

### Preparation 43: tert-Butyl (3S)-3-[ethyl(2-phenoxybenzoyl)amino]pyrrolidine-1-carboxylate

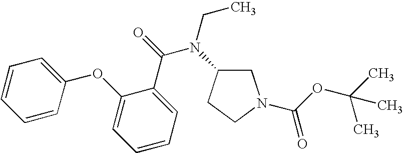

A solution of the amine described in preparation 2 (525 mg, 2.4 mmol) and triethylamine (641 μl, 4.6 mmol), in dichloromethane (5 ml), was added to a solution of 2-phenoxybenzoyl chloride (Tetrahedron (1988), 44(18), 5857-60) (803 mg, 3.5 mmol), in dichloromethane (5 ml), and the reaction mixture was stirred at room temperature for 18 hours. The solution was then evaporated under reduced pressure. The resulting residue was re-dissolved in ether (40 ml), and washed with 10% citric acid solution (50 ml), followed by 2M sodium hydroxide solution (50 ml). The organic solution was dried over magnesium sulfate and concentrated under reduced pressure. The resulting residue was purified by column chromatography on silica gel using an elution gradient of dichloromethane:methanol:0.88 ammonia (100:0:0 to 98:1.8:0.2) to afford the title compound, 943 mg, 93%.

¹HNMR (CD₃OD, 400 MHz): 1.03-1.19 (m, 3H), 1.42 (2×s, 9H), 1.90-2.34 (m, 2H), 3.08-3.62 (m, 6H), 4.22-4.34, 4.55-4.62 (2×m, 1H), 6.92-7.02 (m, 3H), 7.11-7.26 (m, 2H), 7.35-7.43 (m, 4H).

### Preparation 44: tert-Butyl (3S)-3-[isobutyl(2-phenoxybenzoyl)amino]pyrrolidine-1-carboxylate

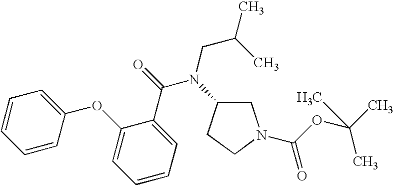

2-Phenoxybenzoyl chloride (Tetrahedron (1988), 44(18), 5857-60) (576 mg, 2.48 mmol) was added to a solution of N-methyl morpholine (417 mg, 4.13 mmol) and the compound described for preparation 3 (500 mg, 2.06 mmol), in toluene (40 ml). 4-(Dimethylamino)pyridine (100 mg, 0.82 mmol) was added, and the reaction mixture was stirred at 60° C. for 18 hours. The mixture was cooled and washed with 10% citric acid solution (3×15 ml), followed by 1M sodium hydroxide (3×15 ml) and water. The organic layer was dried over magnesium sulfate and concentrated in vacuo. The crude product was purified by column chromatography on silica gel eluting with dichloromethane:methanol (100:0 to 95:5, by volume) to yield the title product (770 mg, 85%).

¹HNMR (CD₃OD. 400 MHz): 0.74-0.83 (m, 2.5H), 0.89-0.93 (m, 3.5H), 1.42-1.44 (m, 9H), 1.82-2.15, 2.18-2.29 (2×m, 3H), 3.03-3.20 (m, 2H), 3.26 (m, 1H), 3.43-3.61 (m, 3H), 4.17-4.37 (m, 1H), 6.92-6.98 (m, 2H), 7.04 (m, 1H), 7.14 (m, 1H), 7.23 (m, 1H), 7.35-7.42 (m, 4H); MS APCI⁺ m/z 439 [MH]⁺.

### Preparation 45: tert-butyl (3S)-3-{[2-(ethylthio)benzoyl](isobutyl)amino}pyrrplidine-1-carboxylate

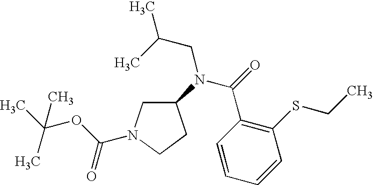

The title compound was prepared using a similar method to that described for preparation 11 using tert-butyl (3S)-3-(isobutylamino)pyrrolidine-1-carboxylate (0.24 g) (preparation 3), the compound described in preparation 34 (0.34 g, 2.0 mmol, 2 eq.), 2,4,6-Tripropyl-[1,3,5,2,4,6]trioxa triphosphinane 2,4,6-trioxide (50% in ethyl acetate, 1.3 ml, 2.0 mmol, 2 eq) and triethylamine (558 μL, 4.0 mmol, 4 eq.), in toluene (5 ml). After work-up, the solvent was removed under reduced pressure and the resulting residue was purified by chromatography on silica gel, using a gradient of methylene chloride changing to methylene chloride:methanol:ammonia (100:10:1 by volume), affording the title compound as an oil (0.40 g, 98%).

LCMS ELSD-APCI⁺ m/z 429 [MNa]⁺

### Preparation 46: tert-butyl (3S)-3-{(cyclopropylmethyl)[2-(ethylthio)benzoyl]amino}pyrrolidine-1-carboxylate

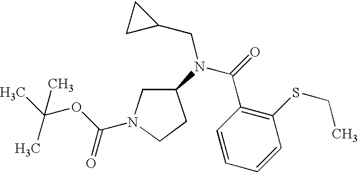

The title compound was prepared using a similar method to that described for preparation 11 using tert-butyl (3S)-3-[(cyclopropylmethyl)amino]pyrrolidine-1-carboxylate (0.24 g, 1.0 mmol, 1 eq.), 2-(ethylthio)benzoic acid (2.0 mmol, 2 eq.) (preparation 34), 2,4,6-tripropyl-1,3,5,2,4,6-trioxatriphosphinane 2,4,6-trioxide (T3P) (50% in ethyl acetate, 1.3 ml, 2.0 mmol, 2 eq) and triethylamine (558 μl, 4.0 mmol, 4 eq.), in toluene (5 ml). After work-up, the solvent was removed under reduced pressure and the resulting residue was purified by chromatography on silica gel using a gradient of methylene chloride changing to methylene chloride:methanol:ammonia (100:10:1 by volume), affording the title compound as an oil (0.38 g, 93%).

LCMS ELSD-APCI⁺ m/z 349 [MH-isobutylene]⁺, 427 [MNa]⁺

### Preparation 47: tert-butyl (3S)-3-[(2-cyclopentylbenzoyl)(cyclopropylmethyl)amino]pyrrolidine-1-carboxylate

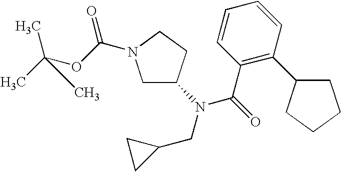

The title compound was prepared using a similar method to that described for preparation 49 using methylene chloride (3 ml) as solvent, tert-butyl (3S)-3-[(cyclopropylmethyl)amino]pyrrolidine-1-carboxylate (0.12 g, 0.48 mmol, 1 eq.) (preparation 13), 2-cyclopentyl-benzoic acid (0.53 mmol, 1.1 eq.) (Journal of Medicinal Chemistry (1964), 7(3), 251-5) and triethylamine (0.133 mL; 0.96 mmol, 2 eq.). After completion of the reaction, methylene chloride (30 ml) was added and the solution was washed with 2N aqueous sodium hydroxide (50 ml) followed by drying over magnesium sulfate. The solvent was removed under reduced pressure and the residue was purified by chromatography on silica gel using a gradient of methylene chloride changing to methylene chloride:methanol:ammonia (100:10:1 by volume), affording the title compound as an oil (0.052 g, 26%).

LCMS ELSD-APCI⁺ m/z 413 [MH]⁺

### Preparation 48: tert-butyl (3S)-3-{cyclohexyl[2-(methylthio)benzoyl]amino}pyrrolidine-1-carboxylate

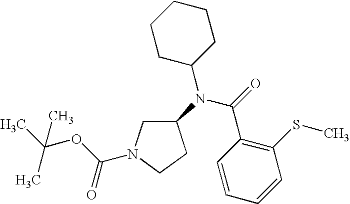

The title compound was prepared using a similar method to that described for preparation 11 using tert-butyl (3S)-3-(cyclohexylamino)pyrrolidine-1-carboxylate (0.268 g, 1.0 mmol, 1 eq.) (preparation 30), commercially available 2-(methylthio)benzoic acid (0.336 g, 2.0 mmol, 2 eq.), 2,4,6-Tripropyl-[1,3,5,2,4,6]trioxa triphosphinane 2,4,6-trioxide (50% in ethyl acetate, 1.3 ml, 2.0 mmol, 2 eq) and triethylamine (558 μL, 4.0 mmol, 4 eq.), in toluene (5 ml). After work-up, the solvent was removed under reduced pressure and the resulting residue was purified by chromatography on silica gel using a gradient of methylene chloride changing to methylene chloride:methanol:ammonia (100:10:1 by volume), affording the title compound as clear oil (0.102 g, 24%).

LCMS ELSD-APCI⁺ m/z 363 [MH-isobutylene]⁺, 419 [MH]⁺

### Preparation 49: tert-Butyl (3S)-3-[(5-fluoro-2-phenoxybenzoyl)(propyl)amino]pyrrolidine-1-carboxylate

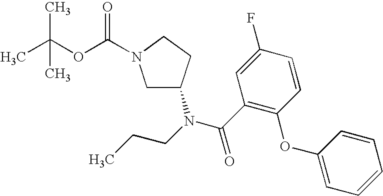

Oxalyl chloride (0.22 ml, 2.56 mmol), followed by N,N-dimethylformamide (5 μl, 0.05 mmol), were added to the carboxylic acid described in preparation 33 (237 mg, 1.02 mmol), in dichloromethane (3 ml). The reaction mixture was stirred for 1 hour at room temperature then the solvent was evaporated. The resulting residue was dissolved in toluene and added to a solution of the amine described in preparation 6 (179 mg, 0.786 mmol), and triethylamine (0.22 ml, 1.57 mmol), in toluene (3 ml). The reaction mixture was stirred at room temperature for 72 hours, followed by 4 hours at 65° C. The solution was filtered through a hydrophobic cartridge and the solvent was removed in vacuo. The crude material was purified by column chromatography using an ISCO® silica gel cartridge eluting with a gradient of pentane:ethyl acetate (100:0 to 0:100) to afford the title compound, 166 mg, 48%.

¹HNMR (400 MHz, CDCl₃) δ: 0.74 (t, 1H), 0.88 (t, 2H), 1.45 (s, 9H), 1.47-1.71 (m, 2H), 1.88-2.14 (m, 2H), 2.96-3.41 (m, 4H), 3.41-3.69 (m, 2H), 4.27, 4.67 (2m, 1H), 6.93 (m, 3H), 7.06 (m, 3H), 7.31 (t, 2H); MS APCI+ m/z 387[MH-isobutylene]⁺, 343[MH-BOC]⁺

### Preparation 50: tert-Butyl (3S)-3-[(2-phenoxybenzoyl)(propyl)amino]pyrrolidine-1-carboxylate

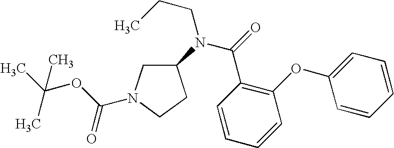

The title compound was prepared as a colourless gum in 83% yield from the compound described in preparation 6 and 2-phenoxybenzoyl chloride, following a similar procedure to that described for preparation 43, except ethyl acetate:pentane was used as the column eluant.

¹HNMR (CDCl₃, 400 MHz): 0.70, 0.89 (2xt, 3H), 1.44 (2×s, 9H), 1.50-1.72 (mm 1H), 1.86-2.10 (m, 2H), 3.01-3.36 (m, 5H), 3.42-3.68 (m, 2H), 4.26, 4.74 (2×m, 1H), 6.91 (d, 1H), 6.98 (d, 2H), 7.07-7.18 (m, 2H), 7.29-7.34 (m, 4H); LCMS ELSD-APCI⁺ m/z 447 [MNa]⁺

### Preparation 51: tert-Butyl (3S)-3-[cyclobutyl(2-phenoxybenzoyl)amino]pyrrolidine-1-carboxylate

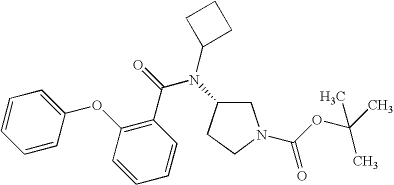

The title compound was prepared as a clear oil in 68% yield from the compound described in preparation 31 and 2-phenoxybenzoyl chloride, following a similar procedure to that described for preparation 43, except ethyl acetate:pentane was used as the column eluant.

¹HNMR (CDCl₃, 400 MHz): 1.44 (2×s, 9H), 1.92-2.00 (m, 2H), 2.09-2.20 (m, 3H), 2.48 (m, 1H), 2.83 (m, 1H), 3.13 (m, 1H), 3.30 (m, 1H), 3.47-3.64 (m, 2H), 3.86 (m, 1H), 4.05 (m, 1H), 4.23 (m, 1H), 6.92-6.96 (m, 3H), 7.07 (m, 1H), 7.17 (m, 1H), 7.29-7.34 (m, 4H); MS APCI⁺ m/z 437 [MH]⁺.

### Preparation 52: tert-Butyl (3S>3-[cyclopentyl(2-phenoxybenzoyl)amino]pyrrolidine-1-carboxylate

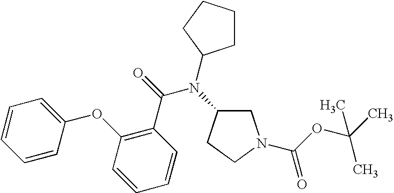

The title compound was prepared from the compound described in preparation 29 and 2-phenoxybenzoyl chloride, following a similar procedure to that described for preparation 43, except that the compound was not purified by column chromatography.

MS APCI⁺ m/z 451 [MH]⁺.

### Preparation 53: tert-Butyl (3S)-3-{isobutyl[(2-phenoxypyridin-3-yl)carbonyl]amino}pyrrolidine-1-carboxylate

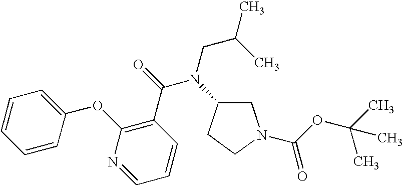

2-Phenoxypyridine-3-carbonyl chloride (463 mg, 1.98 mmol) was added to a solution of the compound described in preparation 3 (400 mg, 1.65 mmol) and N-methyl morpholine (333 mg, 3.30 mmol), in toluene (30 ml), and the reaction mixture was stirred at 60° C. for 18 hours. 4-(Dimethylamino)pyridine (50 mg, 0.4 mmol), additional 2-phenoxypyridine-3-carbonyl chloride (192 mg, 0.83 mmol) and N-methyl morpholine (167 mg, 1.65 mmol) were added, and the reaction mixture was stirred at 60° C. for a further 24 hours. It was then washed with 10% citric add solution (3×), 1M sodium hydroxide solution (3×), dried over sodium sulfate and concentrated under reduced pressure. The resulting residue was purified by column chromatography on silica gel using an elution gradient of dichloromethane:methanol (100:0 to 98:2) to afford the title compound, 540 mg.

¹HNMR (CD₃OD, 400 MHz): 0.82, 0.98 (2×m, 6H), 1.42 (s, 9H), 1.82-2.60 (m, 3H), 3.03-3.38 (m, 3H), 3.42-3.75 (m, 3H), 4.30-4.40 (m, 1H), 7.12 (m, 2H), 7.20 (m, 2H), 7.40 (m, 2H), 7.82 (m, 1H), 8.18 (m, 1H); MS APCI+ m/z 462 [MNa]⁺.

### Preparation 54: tert-Butyl (3S)-3-{(cyclobutylmethyl)[2-(ethylthio)benzoyl]amino}pyrrolidine-1-carboxylate

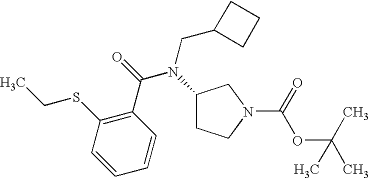

The compound described in preparation 34 (236 mg, 1.3 mmol) was added to a solution of the amine described in preparation 8 (300 mg, 1.18 mmol), in toluene (5 ml), at room temperature. Triethylamine (0.5 ml, 3.54 mmol) was then added, followed by 2,4,6-tripropyl-1,3,5,2,4,6-trioxa triphosphinane 2,4,6-trioxide (50% w/w in ethyl acetate, 2.3 ml, 3.54 mmol). The reaction mixture was stirred at 50° C. for 72 hours, after which time it was allowed to cool to room temperature and washed with 20% aqueous potassium carbonate and 10% citric acid. The organic phase was dried over magnesium sulfate and concentrated in vacuo. The crude product was purified by column chromatography on silica gel eluting with pentane:ethyl acetate (80:20 to 50:50, by volume) to yield the title product, 495 mg.

MS APCI⁺ m/z 419 [MH]⁺ 319 [MH-Boc]⁺

### Preparation 55: tert-butyl (3S)-3-{[(1-methylcyclobutyl)methyl][2-(methylthio)benzoyl]amino}pyrrolidine-1-carboxylate

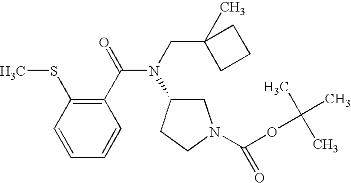

The title compound was prepared by a similar method to that described for preparation 43 using the amine described in preparation 12 and 2-(methylthio)benzoyl chloride to produce the desired product, 246 mg (59%) as a colourless oil.

MS APCI⁺ m/z 419 [MH]⁺

### Preparation 56: tert-butyl (3S)-3-{[(1-methylcyclobutyl)methyl][2-(ethylthio)benzoyl]amino}pyrrolidine-1-carboxylate

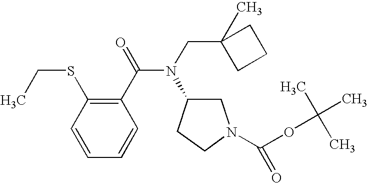

The title compound was prepared by a similar method to that described for preparation 49 using the amine described for preparation 12 and 2-(ethylthio)benzoic acid (preparation 34) to produce the desired product, 263 mg (61%) as a colourless oil.

MS APCI⁺ m/z 433 [MH]⁺

### Preparation 57: tert-butyl (3S)-3-{[2-(ethylthio)benzoyl][(1-methylcyclopropyl)methyl]amino}pyrrolidine-1-carboxylate

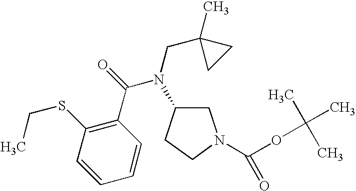

The title compound was prepared by a similar method to that described for preparation 49 using the amine described in preparation 17 and 2-(ethylthio)benzoic acid (preparation 34) to produce the desired product, 362 mg (73%) as a colourless oil. MS APCI⁺ m/z 419 [MH]⁺

### Preparation 58: Tert-butyl (3S)-3-{(cyclobutylmethyl)[2-(isopropylthio)benzoyl]amino}pyrrolidine-1-carboxylate

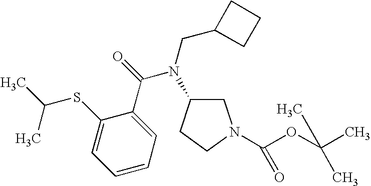

A solution of 2-(isopropylthio)benzoyl chloride (322 mg, 1.5 mmol) (prepared by a similar method to that described for preparation 49 from the corresponding acid, JACS (1949), 71 4062-6) in dichloromethane (2 ml), was added to a solution of tert-butyl (3S)-3-[(cyclobutylmethyl)amino]pyrrolidine-1-carboxylate (preparation 8) (254 mg, 1.0 mmol) and triethylamine (0.279 ml, 0.2 mmol) in dichloromethane (5 ml), and the reaction mixture was stirred at room temperature for 20 hours. It was then diluted with dichloromethane (30 ml), washed with 2M sodium hydroxide (60 ml), dried over magnesium sulfate, filtered and then the solvent was evaporated. The crude material was purified by column chromatography over silica gel eluting with a gradient of 100% dichloromethane to 99:1:0.1 dichloromethane: methanol: ammonia. The title compound was obtained as a clear oil (219 mg, 0.51 mmol, 51%).

LRMS m/z ELSD-APCI⁺ 455 [MNa]⁺.

### Preparation 59: tert-butyl (3S)-3-[(2-phenoxybenzoyl)(phenyl)amino]pyrrolidine-1-carboxylate

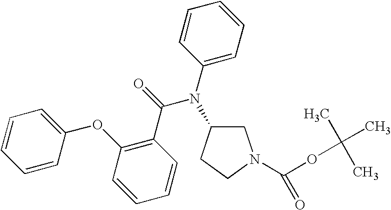

The title compound was prepared by a similar method to that described for preparation 43 using the amine described in preparation 22 and 2-phenoxybenzoyl chloride (Tetrahedron (1988), 44(18), 5857-60) to produce the desired product, 563 mg (98%) as a colourless oil.

¹HNMR (CDCl₃. 400 MHz) δ: 1.40 (s, 9H), 1.83 (m, 1H), 2.14 (m, 1H), 3.06-3.31 (m, 3H), 3.76 (m, 1H), 5.31 (m, 1H), 6.56 (d, 1H), 6.86 (t, 1H), 6.94 (d, 2H), 7.01 (t, 1H), 7.15 (m, 7H), 7.33 (t, 2H); MS APCI⁺ m/z 459 [MH]⁺

### Preparation 60: Tert-butyl (3S)-3-{cyclopentyl[2-(ethylthio)benzoyl]amino}pyrrolidine-1-carboxylate

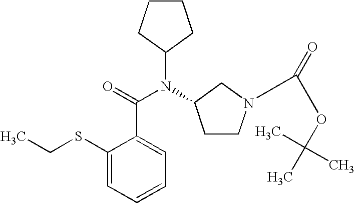

To a solution of 2-(ethylthio)benzoic acid (208 mg, 1.14 mmol) (preparation 34) in dichloromethane (3 ml) was added oxalyl chloride (0.25 ml, 2.85 mmol) and dimethylformamide (0.005 ml). The reaction mixture was stirred at room temperature for 0.75 hours and the solvent was then removed in vacuo. The acid chloride was redissolved in toluene and added dropwise to a solution of tert-butyl (3S)-3-(cyclopentylamino)pyrrolidine-1-carboxylate (223 mg, 0.876 mmol) (preparation 29) and triethylamine (0.25 ml, 1.75 mmol) in toluene (3 ml). The reaction mixture was then stirred at room temperature for 18 hours, followed by heating at 65° C. for 4 hours. The solution was then filtered through a hydrophobic cartridge and the solvent was removed in vacuo. The crude material was purified by column chromatography using an ISCO® silica cartridge eluting with a gradient of 0-100% ethyl acetate: pentane. The title compound was obtained as a colourless gum (224 mg, 0.53 mmol, 60%).

¹HNMR (400 MHz, CDCl₃): δ 0.83-1.38 (m, 9H), 1.45 (s, 9H), 1.50-2.54 (m, 4H), 2.81-3.01 (m, 2H), 3.02-4.07 (m, 6H), 7.11 (d, 1H), 7.21 (t, 1H), 7.29 (t, 1H), 7.37 (d, 1H); LCMS m/z ELSD-APC⁺ 419 [MH]⁺.

### Preparation 61: tert-butyl (3S)-3-{(cyclobutylmethyl)[2-(1,1-difluoroethyl)benzoyl]amino}pyrrolidine-1-carboxylate

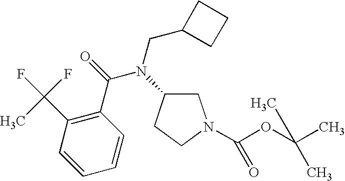

2-(1,1-Difluoro-ethyl)-benzoyl chloride (0.38 g, 1.31 mmol, 1 eq) (preparation 37), the compound described in preparation 8 (0.66 g, 2.58 mmol, 1.2 eq.) and triethylamine (0.33 g, 3.23 mmol, 1.5 eq.) were mixed in methylene chloride (30 ml). After stirring for 5 hours at room temperature, the solvent was removed under reduced pressure. The resulting residue was dissolved in ethyl acetate and the solution was washed three times with a 10% aqueous solution of citric acid, twice with brine, and then dried over magnesium sulfate. The solvent was removed under reduced pressure and the residue was purified by chromatography on silica gel using a gradient of methylene chloride changing to methylene chloride:methanol:ammonia (100:10:1 by volume) affording the title compound as an oil (620 mg, 68%).

LCMS ELSD-APCI⁺ m/z 423 [MH]⁺.

### EXAMPLE 1

### N-Ethyl-2-phenoxy-N-[(3S)-pyrrolidin-3-yl]benzamide hydrochloride

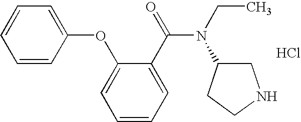

Hydrogen chloride (4M in 1,4-dioxane, 12 ml, 48 mmol) was added to a solution of the compound described in preparation 43 (943 mg, 2.30 mmol) in dichloromethane (10 ml), and the reaction mixture was stirred at room temperature for 3 hours. The solution was then evaporated under reduced pressure, the residue was triturated with ether (20 ml) and the mixture was evaporated in vacuo to afford the title compound as a white foam.

¹HNMR (CD₃OD, 400 MHz): 1.18 (t, 3H), 2.04-2.20, 2.38-2.57 (2×m, 2H), 3.16-3.25 (m, 1H), 3.30-3.52 (m, 3H), 3.63-3.79 (m, 2H), 4.22-4.35 (m, 1H), 6.94-7.02 (m, 3H), 7.16 (m, 1H), 7.22 (m, 1H), 7.31-7.45 (m, 4H); MS APCI+ m/z 311 [MH]⁺.

### EXAMPLE 2

### N-Isobutyl-2-phenoxy-N-[(3S)-pyrrolidin-3-yl]benzamide

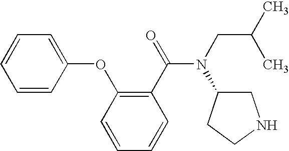

The compound described in preparation 44 (768 mg, 1.75 mmol) was dissolved in dichloromethane (10 ml), under nitrogen, and trifluoroacetic acid (10 ml) was added dropwise at 0° C. The reaction mixture was stirred at room temperature for 3 hours after which time the solution was evaporated, azeotroped with toluene and concentrated in vacuo. The resulting residue was taken up in ethyl acetate and washed with 1M sodium hydroxide solution (3×20 mL). The organic phase was dried over magnesium sulfate and concentrated in vacuo to yield the title product (542 mg, 91%).

¹HNMR (CD₃OD, 400 MHz): 0.78, 0.82, 0.92 (3xd, 6H), 1.79-1.98 (m, 2H), 2.18 (m, 1H), 2.66-2.78 (m, 1.5H), 2.83-3.07 (m, 2H), 3.13-3.28 (m, 2H), 3.42 (m, 0.5H), 3.94 (m, 0.5H), 4.28 (m, 0.5H), 6.91-6.98 (m, 2H), 7.03 (m, 1H), 7.13 (m, 1H), 7.22 (m, 1H), 7.33-7.43 (m, 4H); MS APCI⁺ m/z 339 [MH]⁺.

### EXAMPLE 3

### N-Cyclobutyl-2-phenoxy-N-[(3S)-pyrrolidin-3-yl]benzamide hydrochloride

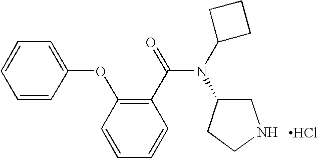

The compound described in preparation 51 (504 mg, 1.15 mmol) was dissolved in a solution of 4M hydrogen chloride in 1,4-dioxane (5 ml). The reaction mixture was stirred at room temperature for 20 minutes after which time it was concentrated in vacuo. The resulting residue was azeotroped with toluene and ether, and then it was triturated with ether to yield the title product (366 mg, 85%) as a white solid.

¹HNMR (CD₃OD, 400 MHz): 1.55 (m, 1H), 170 (m, 1H), 1.86-2.06 (m, 1H), 2.02 (m, 1H), 2.14-2.23 (m, 4H), 2.37 (m, 0.5H), 3.04 (m, 0.5H), 3.16 (m, 1H), 3.46 (m, 0.5H), 3.67 (m, 1H), 3.76 (m, 0.5H), 4.28 (m, 1H), 4.58 (m, 1H), 6.95 (d, 2H), 7.02 (d, 1H), 7.11 (m, 1H), 7.26 (m, 1H), 7.32-7.38 (m, 3H), 7.47 (t, 1H);

MS APCI⁺ m/z 337 [MH]⁺

### EXAMPLE 4

### N-Cyclopentyl-2-phenoxy-N-[(3S)-pyrrolidin-3-yl]benzamide hydrochloride

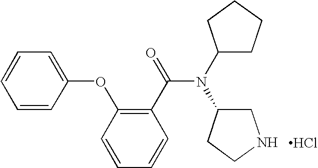

Hydrogen chloride (4M in 1,4-dioxane, 10 ml, 40 mmol) was added to the compound described in preparation 52 (530 mg, 1.18 mmol), and the reaction mixture was stirred at room temperature for 2 hours. It was then concentrated under reduced pressure and the resulting residue was azeotroped with toluene. The crude product was partitioned between dichloromethane and 20% potassium carbonate solution, the layers were separated, and the organic phase was dried over magnesium sulfate and evaporated under reduced pressure. The crude product was purified by column chromatography on silica gel using an elution gradient of dichloromethane:methanol:0.88 ammonia (100:0:0 to 80:20:2). The product was treated with 2M hydrogen chloride in ether, to provide the title compound as a white foam, 235 mg; 52%.

¹HNMR (CD₃OD, 400 MHz): 1.42-1.63 (m, 4H), 1.68-1.81 (m, 3H), 1.88 (m, 1H), 2.10 (m, 0.5H), 2.33 (m, 0.5H), 2.46 (m, 1H), 3.11-3.36 (m, 1.5H), 3.34-3.49 (m, 1H), 3.65-3.80 (m, 1.5H), 4.04 (m, 1H), 4.23 (m, 1H), 6.96-6.99 (m, 3H), 7.12 (m, 1H), 7.25 (t, 1H), 7.33-7.46 (m, 4H); LCMS ELSD-APCI⁺ m/z 351 [MH]⁺.

### EXAMPLE 5

### 2-Phenoxy-N-propyl-N-[(3S)-pyrrolidin-3-yl]benzamide hydrochloride

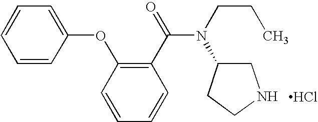

The title compound was prepared from the compound described in preparation 50 by a method similar to that described for example 3 to yield the title product (294 mg, 83%).

¹HNMR (CD₃OD, 400 MHz): 0.79 (t, 3H), 1.52-1.66 (m, 2H), 2.02-2.18, 2.37-2.55 (2×m, 2H), 3.11-3.30 (m, 4H), 3.41-3.50, 3.62-3.79 (2×m, 2H), 4.26 (m, 1H), 6.95-7.00 (m, 3H), 7.14 (t, 1H), 7.24 (t, 1H), 7.34-7.46 (m, 4H); LCMS ELSD-APCI⁺ m/z 325 [MH]⁺.

### EXAMPLE 6

### N-Isobutyl-2-phenoxy-N-[(3S)-pyrrolidin-3-yl]nicotinamide

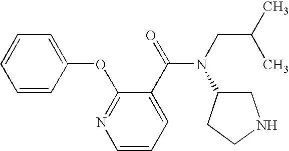

Trifluoroacetic acid (1 ml) was added to an ice-cooled solution of the compound described in preparation 53 (540 mg, 1.23 mmol) in dichloromethane (15 ml), and the reaction mixture was stirred at room temperature for 4 hours. It was then washed with 1M sodium hydroxide solution (3×), dried over sodium sulfate and evaporated under reduced pressure to afford the title compound, 380 mg, 91%.

¹HNMR (CD₃OD, 400 MHz): 0.82, 0.99 (2×m, 6H), 1.81-2.35 (m, 3H), 2.70-2.98 (m, 2H), 3.00-3.18 (m, 2H), 3.19-3.38, 3.42-52 (2×m, 2H), 4.00-4.10, 4.21-4.30 (2×m, 1H), 7.11 (m, 2H), 7.20 (m, 2H), 7.40 (m, 2H), 7.80 (m, 1H), 8.17 (m, 1H); MS APCI+ m/z 340 [MH]⁺.

### EXAMPLE 7

### 5-fluoro-2-phenoxy-N-propyl-N-[(3S)-pyrrolidin-3-yl]benzamide hydrochloride

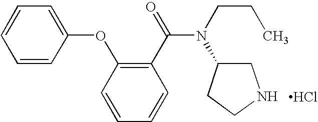

The title compound was prepared from the compound described in preparation 49 by a method similar to that described for example 3 to yield the title product 116 mg, 82%.

¹HNMR (CD₃OD, 400 MHz) δ: 0.81 (t, 3H), 1.60 (m, 2H), 2.01 (m, 1H), 2.29-2.55 (m, 1H), 3.06-3.29 (m, 3H), 3.36-3.81 (m, 3H), 4.24 (m, 1H), 6.94-7.06 (m, 3H), 7.13 (m, 1H), 7.22 (m, 2H), 7.35 (t, 2H); LCMS ELSD-APCI⁺ m/z 343 [MH]⁺.

### EXAMPLE 8

### N-(cyclobutylmethyl)-2-(ethylthio)-N-[(3S)-pyrrolidin-3-yl]benzamide hydrochloride

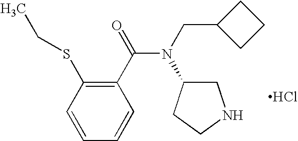

The title compound was prepared from the compound described in preparation 54 by a method similar to that described for example 3 to yield the title product (249 mg)

¹HNMR/CD₃OD, 400 MHz) δ: 1.30 (t, 3H), 1.55 (m, 2H), 1.66 (m, 1H), 1.86 (m, 1H), 1.99 (m, 2H), 2.55 (m, 3H), 3.02 (m, 2H), 3.22 (m, 3H), 3.48 (m, 1H), 3.78 (m, 2H), 4.27 (m, 1H), 7.25-7.35 (m, 2H), 7.44 (m, 1H), 7.51 (m, 1H); MS APCI⁺ m/z 319 [MH]⁺.

### EXAMPLE 9

### N-[(1-methylcyclobutyl)methyl]-2-(methylthio)-N-[(3S)-pyrrolidin-3-yl]benzamide hydrochloride

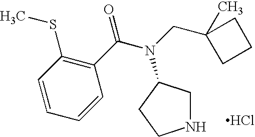

The title compound was prepared by a similar method to that described for example 3 using the protected amine described in preparation 55 to produce the desired product, 196 mg (93%) as a white solid.

¹HNMR (CD₃OD, 400 MHz) δ: 1.11 (s, 3H), 1.50-1.75 (m, 3H), 1.78-2:05 (m, 3H), 2.54 (m, 5H), 3.15-3.39 (m, 3H), 3.51 (m, 1H), 3.80 (m, 2H), 4.30 (m, 1H), 7.25 (m, 2H), 7.43 (m, 2H); MS APCI⁺ m/z 319 [MH]⁺

### EXAMPLE 10

### 2-(ethylthio)-N-[(1-methylcyclobutyl)methyl]-N-[(3S)-pyrrolidin-3-yl]benzamide hydrochloride

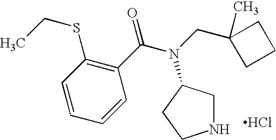

The title compound was prepared by a similar method to that described for example 3 using the protected amine described in preparation 56 to produce the desired product, 216 mg (96%) as a white solid.

¹HNMR(CD₃OD, 400 MHz) δ: 1.11 (s, 3H), 1.29 (t, 3H), 1.44-1.84 (m, 4H), 1.86-2.11 (m, 2H), 2.46-2.67 (m, 2H), 3.01 (m, 2H), 3.10-3.40 (m, 3H), 3.51 (m, 1H), 3.81 (m, 2H), 4.29 (m, 1H), 7.29 (m, 2H), 7.42 (m, 1H), 7.46 (m, 1H); MS APCI⁺ m/z 333 [MH]⁺

### EXAMPLE 11

### 2-(ethylthio)-N-[(1-methylcyclopropyl)methyl]-N-[(3S)-pyrrolidin-3-yl]benzamide hydrochloride

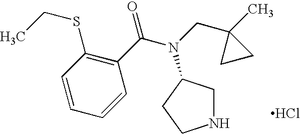

The title compound was prepared by a similar method to that described for example 3 using the protected amine described in preparation 57 to produce the desired product, 280 mg (92%) as a white solid.

¹HNMR(CD₃OD, 400 MHz) δ: 0.17-0.68 (m, 4H), 0.99 (s, 3H), 1.29 (t, 3H), 2.59 (m, 2H), 3.02 (m, 3H), 3.27 (m, 2H), 3.55 (m, 1H), 3.84 (m, 2H), 4.50 (m, 1H), 7.30 (m, 2H), 7.47 (m, 2H); MS APCI⁺ m/z 319[MH]⁺

### EXAMPLE 12

### N-(sec-butyl)-2-phenoxy-N-[(3S)-pyrrolidin-3-yl]benzamide hydrochloride

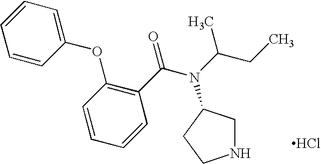

The title compound was prepared by a similar method to that described in example 3 using the protected amine described for preparation 21 to produce the desired product, 329 mg (100%) as a foam.

¹HNMR (CD₃OD, 400 MHz) δ: 0.80 (q, 2H), 0.88 (m, 1H), 1.17 (m, 1H), 1.24 (t, 2H), 1.45-1.70 (m, 2H), 2.10-2.55 (m, 3H), 3.40 (m, 1H), 3.55-3.80 (m, 3H), 4.26 (m, 1H), 6.80 (m, 1H), 7.00 (m, 2H), 7.15 (m, 1H), 7.22 (m, 1H), 7.36 (m, 4H); MS APCI⁺ m/z 339 [MH]⁺

### EXAMPLE 13

### N-(cyclobutylmethyl)-2-phenoxy-N-[(3S)-pyrrolidin-3-yl]benzamide hydrochloride

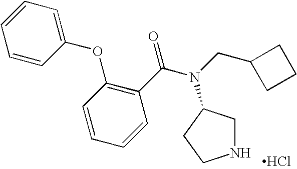

Using a method similar to that described for example 17, the compound described in preparation 25 (0.411 g, 0.9 mmol, 1 eq) afforded the title compound as a foam (0.300 g, 85%).

¹HNMR(CD₃OD, 400 MHz) δ: 1.45-1.75 (m, 3H), 1.80-2.13 (m, 4H), 2.27-2.65 (m, 2H), 3.05-3.30 (m, 4H with MeOH peak), 3.40 (m, 1H), 3.55-3.80 (m, 1H), 4.18 (m, 1H), 6.99 (m, 3H), 7.15 (t, 1H), 7.27 (t, 1H), 7.43 (multiplets, 4H); LCMS APCI+ m/z 351 (MH⁺)

### EXAMPLE 14

### 2-phenoxy-N-phenyl-N-[(3S)-pyrrolidin-3-yl]benzamide hydrochloride

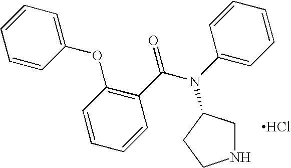

The title compound was prepared by a similar method to that described for example 3 using the protected amine described in preparation 59 to produce the desired product, 429 mg (69%) as a white gum.

¹HNMR (CD₃OD, 400 MHz) δ: 2.29 (m, 1H), 2.48 (m, 1H), 3.27 (m, 1H), 3.62 (m, 3H), 4.83 (m, 1H), 6.57 (d, 1H), 6.88 (d, 2H), 6.98 (t, 1H), 7.15 (t, 2H), 7.22 (m, 5H), 7.35 (m, 3H); MS APCI⁺ m/z 359 [MH]⁺

### EXAMPLE 15

### N-(cyclobutylmethyl)-2-(methylthio)-N-[(3S)-pyrrolidin-3-yl]benzamide hydrochloride

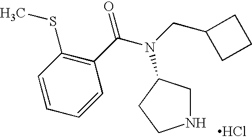

The compound described in preparation 20 (0.30 g, 0.75 mmol, 1 eq) was dissolved in 4N hydrogen chloride (3 ml) in dioxane. The solution was stirred for 1 hour at room temperature and then the solvent was evaporated under reduced pressure. The resulting residue was dissolved in toluene and then the solvent was removed under reduced pressure, affording the title compound as a foam (0.24 g, 95%).

¹HNMR(CD3OD, 400 MHz) δ: 1.50-1.76 (multiplets, 3H), 1.85 (m, 1H), 1.99 (m, 2H), 2.43-2.70 (multiplets, 6H), 3.22 (m, 3H), 3.50 (m, 1H), 3.78 (m, 2H), 4.26 (m, 1H), 7.07-7.33 (m, 2H plus toluene), 7.43 (m, 2H); LCMS APCI+ m/z 305 [MH]⁺

### EXAMPLE 16

### N-(cyclobutylmethyl)-2-(1,1-difluoroethyl)-N-[(3S)-pyrrolidin-3-yl]benzamide hydrochloride

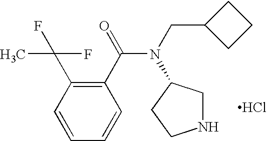

The compound described in preparation 61 (0.58 g, 1.37 mmol, 1 eq) was dissolved in methylene chloride (20 ml) and cooled to 0° C. Trifluoroacetic acid (20 ml) was added and the solution was stirred at 0° C. for 3 hours. The solvent was then removed under reduced pressure and the residue was redissolved in ethyl acetate (30 ml). This solution was washed with 1N aqueous sodium hydroxide and dried over magnesium sulfate. The solvent was removed under reduced pressure and the residue was purified by chromatography on silica gel using a gradient of methylene chloride changing to methylene chloride:methanol:ammonia (100:10:1 by volume), affording the title compound as an oil (240 mg, 54%).

¹HNMR (CD₃OD, 400 MHz) [symbol] : 1.50 (m, 1H), 1.60-2.30 (m, 11H), 2.50-3.10 (m, 5H), 3.40 (m, 1H), 4.0 (m, 1H), 7.25 (m, 1H), 7.50-7.60 (m, 3H); LCMS ELSD-APCI⁺ m/z 323 [MH]⁺.

### EXAMPLE 17

### 2-(ethylthio)-N-isobutyl-N-[(3S)-pyrrolidin-3-yl]benzamide hydrochloride

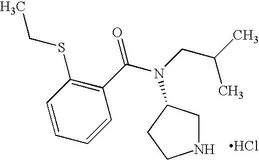

The compound described in preparation 45 (0.40 g, 0.98 mmol, 1 eq) was dissolved in methylene chloride (5 ml). 4N Hydrogen chloride in dioxane (4.9 ml, 19.6 mmol, 20 eq.) was added, and the solution was stirred at room temperature for 3 hours. The solvent was then removed under reduced pressure, ether (30 ml) was added and the solution was then evaporated to dryness, affording the title compound as a white foam (0.33 g, 97%).

¹HNMR (CD₃OD, 400 MHz) [symbol] 0.79 (t, 6H), 1.27 (t, 3H), 1.86 (m, 1H), 2.43-2.69 (b, 2H), 2.80-3.06 (b, 3H), 3.06-3.27 (m, 2H), 3.52 (t, 1H), 3.81 (m, 2H), 4.33 (m, 1H), 7.28 (m, 2H), 7.44 (t, 1H), 7.49 (d, 1H); LCMS ELSD-APCI⁺ m/z 307 [MH]⁺

### EXAMPLE 18

### N-(cyclopropylmethyl)-2-(ethylthio)-N-[(3S)-pyrrolidin-3-yl]benzamide hydrochloride

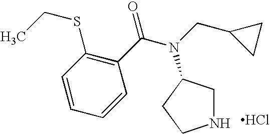

The title compound was prepared from the compound described in preparation 46 (0.38 g, 0.9 mmol, 1 eq) by a method similar to that described for example 17 to yield the title product as a foam (0.28 g, 87%).

¹HNMR (CD₃OD, 400 MHz) [symbol] 0.12 (b, 2H), 0.51 (b, 2H), 0.96 (b, 1H), 1.26 (t, 3H), 2.42-2.64 (b, 2H), 2.90-3.17 (b, 4H), 3.26 (q, 1H), 3.75 (m, 1H), 3.82 (m, 2H), 4.43 (m, 1H), 7.29 (m, 2H), 7.43 (t, 1H), 7.50 (d, 1H); LCMS ELSD-APCI⁺ m/z 305 [MH]⁺

### EXAMPLE 19

### 2-cyclopentyl-N-(cyclopropylmethyl)-N-[(3S)-pyrrolidin-3-yl]benzamide hydrochloride

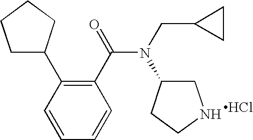

The title compound was prepared from the compound described in preparation 47 (0.05 g, 0.12 mmol, 1 eq.) by a method similar to that described for example 17 to yield the title product as a foam (0.038 g, 87%).

¹HNMR (CD₃OD, 400 MHz) [symbol] : 0.11 (m, 2H), 0.55 (m, 2H), 0.89 (m, 1H), 1.48-1.79 (m, 4H), 1.80-1.96 (m, 2H), 1.98-2.14 (m, 2H), 2.47 (m, 1H), 2.62 (m, 1H), 2.90-3.31 (m, 4H), 3.43 (m, 1H), 3.63-3.88 (m, 2H), 4.44 (m, 1H), 7.15-7.29 (m, 2H), 7.41 (m, 2H); LCMS ELSD-APCI⁺ m/z 313 [MH]⁺

### EXAMPLE 20

### N-cyclohexyl-2-(methylthio)-N-[(3S)-pyrrolidin-3-yl]benzamide hydrochloride

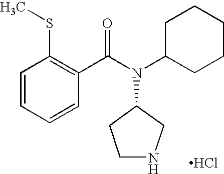

The title compound was prepared from the compound described in preparation 48 (0.102 g, 0.24 mmol, 1 eq.) by a method similar to that described for example 17 to yield the tide product as a foam (0.073 g, 84%).

¹HNMR (CD₃OD, 400 MHz) [symbol] : 0.82-1.19 (m, 3H), 1.23-1.37 (m, 1H), 1.45-1.84 (m, 5H), 1.86-2.02 (m, 1H), 2.40-2.60 (m, 4H), 3.20 (m, 2H), 3.45 (q, 1H), 3.55-3.68 (m, 1H), 3.71 (m, 1H), 3.81 (m, 1H), 4.42 (m, 1H), 7.20 (m, 1H), 7.28 (t, 1H), 7.39-7.53 (m, 2H); LCMS ELSD-APCI⁺ m/z 319 [MH]⁺

### EXAMPLE 21

### N-(cyclobutylmethyl)-2-(isopropylthio)-N-[(3S)-pyrrolidin-3-yl]benzamide hydrochloride

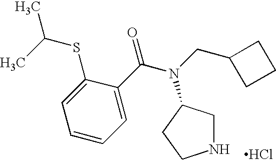

A solution of 4M hydrogen chloride in dioxane (2.5 ml, 10.2 mmol) was added to a solution of compound described in preparation 58 (219 mg, 0.51 mmol) in dichloromethane (5 ml), and the resulting mixture was stirred at room temperature for 3 hours. The solvent was then evaporated to yield the title compound as the hydrochloride salt (183 mg, 0.50 mmol, 100%).

¹HNMR (CD₃OD, 400 MHz) δ: 1.25 (d, 6H), 1.36-1.73 (m, 3H), 1.74-2.09 (m, 3H), 2.31-2.63 (m, 2H), 3.07-3.30 (m, 4H), 3.51 (m, 2H), 3.63-3.84 (m, 2H), 4.26 (m, 1H), 7.29 (b, 1H), 7.35 (t, 1H), 7.44 (t, 1H), 7.53 (d, 1H); LRMS m/z ELSD-APCI⁺ 333 [MH]⁺.

### EXAMPLE 22

### N-cyclopentyl-2-(ethylthio)-N-[(3S)-pyrrolidin-3-yl]benzamide hydrochloride

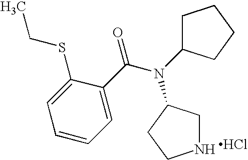

A solution of the compound described in preparation 60 (224 mg, 0.53 mmol) in 4M hydrogen chloride in dioxane (3 ml) was stirred at room temperature for 2 hours. The solvent was then evaporated and the resulting residue was azeotroped with toluene (2×10 ml), dichloromethane (2×10 ml) and diethyl ether (10 ml), to yield the title compound (169 mg, 0.47 mmol, 88%).

¹HNMR (CD₃OD, 400 MHz) δ: 1.28 (t, 3H), 1.33-1.81 (m, 7H), 1.91 (m, 1H), 2.40-2.67 (m, 2H), 3.00 (m, 2H), 3.24 (m, 1H), 3.51 (m, 1H), 3.69-3.87 (m, 3H), 4.29 (m, 1H), 7.22 (m, 1H), 7.31 (t, 1H), 7.43 (t, 1H), 7.51 (m, 1H); LRMS m/z ELSD-APCI⁺ 319 [MH]⁺.

### EXAMPLE 23

### N-cyclopentyl-2-(methylthio)-N-[(3S)-pyrrolidin-3-yl]benzamide hydrochloride

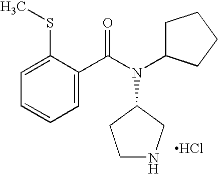

A solution of the compound described in preparation 24 (289 mg, 0.71 mmol) in 4M hydrogen chloride, in dioxane (3 ml), was stirred at room temperature for 2 hours. The solvent was then evaporated and the resulting residue was azeotroped with toluene (2×10 ml), dichloromethane (2×10 ml) and diethyl ether (10 ml), to yield the title compound (216 mg, 0.63 mmol, 88%).

¹HNMR (CD₃OD, 400 MHz) δ: 1.34-1.81 (m, 7H), 1.83-1.99 (m, 1H), 2.40-2.66 (m, 2H), 2.50 (s, 3H), 3.25 (m, 1H), 3.52 (m, 1H), 3.69-3.87 (m, 3H), 4.30 (m, 1H), 7.21 (m, 1H), 7.28 (m, 1H), 7.46 (m, 2H); LRMS m/z ELSD-APCI⁺ 305 [MH]⁺.

### EXAMPLE 24

All of the following compounds exhibited an NRI Ki and/or an SRI Ki of less than 200 nM when tested using the biological activity assay described:
N-(cyclopropylmethyl)-2-phenoxy-N-[(3S)-pyrrolidin-3-yl]benzamide (MS APCI+ m/z 337 [MH]⁺);
N-[(1-methylcyclopropyl)methyl]-2-phenoxy-N-[(3S)-pyrrolidin-3-yl]benzamide (MS APCI+ m/z 351 [MH]⁺);
N-cyclohexyl-2-phenoxy-N-[(3S)-pyrrolidin-3-yl]benzamide (MS APCI+ m/z 365 [MH]⁺);
2-phenoxy-N-[(3S)-pyrrolidin-3-yl]-N-(tetrahydro-2H-pyran-4-yl)benzamide (MS APCI+ m/z 367 [MH]⁺);
2-phenoxy-N-[(3S)-pyrrolidin-3-yl]-N-{[1-(trifluoromethyl)cyclopropyl]methyl}benzamide (MS APCI+ m/z 405 [MH]⁺);
N-cyclobutyl-2-(difluoromethoxy)-N-[(3S)-pyrrolidin-3-yl]benzamide (MS APCI+ m/z 311 [MH]⁺);
2-(difluoromethoxy)-N-[(1-methylcyclopropyl)methyl]-N-[(3S)-pyrrolidin-3-yl]benzamide (MS APCI+ m/z 325 [MH]⁺);
N-cyclobutyl-3-phenoxy-N-[(3S)-pyrrolidin-3-yl]benzamide (MS APCI+ m/z 337 [MH]⁺);
2-benzyl-N-isobutyl-N-[(3S)-pyrrolidin-3-yl]benzamide (MS APCI+ m/z 337 [MH]⁺);
N-cyclobutyl-2-(methylthio)-N-[(3S)-pyrrolidin-3-yl]benzamide (MS APCI+ m/z 291 [MH]⁺);
N-methyl-2-phenoxy-N-[(3S)-pyrrolidin-3-yl]benzamide (MS APCI+ m/z 297 [MH]⁺);
N-cyclobutyl-2-(ethylthio)-N-[(3S)-pyrrolidin-3-yl]benzamide (MS APCI+ m/z 305 [MH]⁺);
N-cyclobutyl-2-cyclopentyl-N-[(3S)-pyrrolidin-3-yl]benzamide (MS APCI+ m/z 313 [MH]⁺);
2-cyclobutyl-N-cyclopentyl-N-[(3S)-pyrrolidin-3-yl]benzamide (MS APCI+ m/z 313 [MH]⁺);
2-cyclobutyl-N-(2,2-dimethylpropyl)-N-[(3S)-pyrrolidin-3-yl]benzamide (MS APCI+ m/z 315 [MH]⁺);
N,2-dicyclopentyl-N-[(3S)-pyrrolidin-3-yl]benzamide (MS APCI+ m/z 327 [MH]⁺);
2-cyclopentyl-N-propyl-N-[(3S)-pyrrolidin-3-yl]benzamide (MS APCI+ m/z 301 [MH]⁺);
2-cyclopentyl-N-(2,2-dimethylpropyl)-N-[(3S)-pyrrolidin-3-yl]benzamide (MS APCI+ m/z 329 [MH]⁺);
N-isopropyl-2-phenoxy-N-[(3S)-pyrrolidin-3-yl]benzamide (MS APCI+ m/z 325 [MH]⁺);
N-(cyclobutylmethyl)-2-cyclopropyl-N-[(3S)-pyrrolidin-3-yl]benzamide (MS APCI+ m/z 299 [MH]⁺);
N-(cyclopropylmethyl)-2-(methylthio)-N-[(3S)-pyrrolidin-3-yl]benzamide (MS APCI+ m/z 291 [MH]⁺);
2-cyclobutyl-N-(cyclopropylmethyl)-N-[(3S)-pyrrolidin-3-yl]benzamide (MS APCI+ m/z 299 [MH]⁺);
2-cyclopropyl-N-(2,2-dimethylpropyl)-N-[(3S)-pyrrolidin-3-yl]benzamide (LC/MS APCI+ m/z 301 [MH]⁺);
N-isobutyl-2-(methylthio)-N-[(3S)-pyrrolidin-3-yl]benzamide (MS APCI+ m/z 293 [MH]⁺);
N-(cyclobutylmethyl)-2-cyclopentyl-N-[(3S)-pyrrolidin-3-yl]benzamide (MS APCI+ m/z 327 [MH]⁺);
2-cyclobutyl-N-(cyclobutylmethyl)-N-[(3S)-pyrrolidin-3-yl]benzamide (MS APCI+ m/z 313 [MH]⁺);
N-(cyclobutylmethyl)-2-(difluoromethoxy)-N-[(3S)-pyrrolidin-3-yl]benzamide (MA APCI+ m/z 325 [MH]⁺);
N-(cyclopentylmethyl)-2-(methylthio)-N-[(3S)-pyrrolidin-3-yl]benzamide (MS APCI+ m/z 319 [MH]⁺);
N-(cyclopentylmethyl)-2-cyclopropyl-N-[(3S)-pyrrdidin-3-yl]benzamide (MS APCI+ m/z 313 [MH]⁺);
2-(methylthio)-N-propyl-N-[(3S)-pyrrolidin-3-yl]benzamide (MS APCI+ m/z 279 [MH]⁺);
N-(2,2-dimethylpropyl)-2-(ethylthio)-N-[(3S)-pyrrolidin-3-yl]benzamide (MS APCI+ m/z 321 [MH]⁺);
2-(ethylthio)-N-propyl-N-[(3S)-pyrrolidin-3-yl]benzamide (MS APCI+ m/z 293 [MH]⁺);
N-(cyclopropylmethyl)-2-(isopropylthio)-N-[(3S)-pyrrolidin-3-yl]benzamide (MS APCI+ m/z 319 [MH]⁺);
2-(methylthio)-N-phenyl-N-[(3S)-pyrrolidin-3-yl]benzamide (MS APCI+ m/z 313 [MH]⁺);
3-fluoro-2-phenoxy-N-propyl-N-[(3S)-pyrrolidin-3-yl]benzamide (MS APCI+ m/z 343 [MH]⁺);
2-(ethylthio)-N-phenyl-N-[(3S)-pyrrolidin-3-yl]benzamide (MS APCI+ m/z 327 [MH]⁺);
N-(sec-butyl)-2-(ethylthio)-N-[(3S)-pyrrolidin-3-yl]benzamide (MS APCI+ m/z 307 [MH]⁺);
N-(cyclobutylmethyl)-2-phenoxy-N-[(3R)-pyrrolidin-3-yl]benzamide (MS APCI+ m/z 351 [MH]⁺);
N-ethyl-2-(4-fluorophenoxy)-N-[(3S)-pyrrolidin-3-yl]benzamide (LC/MS APCL+ m/z 329 [MH]⁺);
N-ethyl-5-fluoro-2-phenoxy-N-[(3S)-pyrrolidin-3-yl]benzamide (LC/MS APCI+ m/z 329 [MH]⁺);
2-(4-fluorophenoxy)-N-propyl-N-[(3S)-pyrrolidin-3-yl]benzamide (MS APCI m/z 343 [MH]⁺);
N-(2-cyclopropylethyl)-2-(methylthio)-N-[(3S)-pyrrolidin-3-yl]benzamide (MS APCI+ m/z 305 [MH]⁺);
N-(2-cyclopropylethyl)-2-(ethylthio)-N-[(3S)-pyrrolidin-3-yl]benzamide (MS APCI+ m/z 319 [MH]⁺);
N-[(1-methylcyclopropyl)methyl]-2-(methylthio)-N-[(3S)-pyrrolidin-3-yl]benzamide (MS APCI+ m/z 305[MH]⁺);
N-cyclohexyl-2-(ethylthio)-N-[(3S)-pyrrolidin-3-yl]benzamide (MS APCI+ m/z 333 [MH]⁺);
N-(2,2-dimethylpropyl)-2-(methylthio)-N-[(3S)-pyrrolidin-3-yl]benzamide (MS APCI+ m/z 307 [MH]⁺);
N-cyclopentyl-2-cyclopropyl-N-[(3S)-pyrrolidin-3-yl]benzamide (LC/MS APCI+ m/z 299 [MH]⁺);
N-(sec-butyl)-2-(methylthio)-N-[(3S)-pyrrolidin-3-yl]benzamide (MS APCI+ m/z 293 [MH]⁺);
N-cyclopropyl-2-phenoxy-N-pyrrolidin-3-yl]benzamide (MS APCI⁺ m/z 323 [MH]⁺);
2-(4-chlorophenoxy)-N-propyl-N-[(3S)-pyrrolidin-3-yl]benzamide (MS APCI+ m/z 359 [MH]⁺);
N-cyclobutyl-2-(phenylthio)-N-[(3S)-pyrrolidin-3-yl]benzamide (MS ES+m/z 353 [MH]⁺);
N-(cyclobutylmethyl)-2-(cyclopropylmethoxy)-N-[(3S)-pyrrolidin-3-yl]benzamide (MS APCI+ m/z 329[MH]⁺);

The NRI Ki and the SRI Ki of the exemplified compounds were determined as described below. A selection of the results is set out below in Table 1. All of the exemplified compounds exhibited an NRI Ki and/or an SRI Ki of less than 200 nM.

TABLE 1

| Compound | SRI Ki (nM) | NRI Ki (nM) |
|---|---|---|
| Example 4 | 70 | 12 |
| Example 7 | 2400 | 9 |
| Example 9 | 20 | 6 |
| Example 14 | 67 | 3 |
| Example 19 | 500 | 16 |
| Example 20 | 17 | 5 |

### Biological Activity

The compounds are tested for biological activity by their ability to inhibit binding of selective radioligands at the human serotonin and noradrenaline transporters (SERT and NET, respectively), using scintillation proximity assay (SPA) technology. The SPA binding is performed using cellular membrane preparations prepared from cell lines expressing human cDNA encoding either SERT or NET (hSERT, hNET), using the radioligands 3H-citalopram and 3H-nisoxetine.

i) Cell Culture Methodology

Human embryonic kidney cells (HEK-293) expressing each transporter are maintained as a continuous culture, using standard cell culture techniques, in 50 mL of growth medium (see Media and Buffers for composition) in 225 cm² flasks, at 37° C. in a humidified atmosphere with 5% CO₂ present. Cells are passaged from a 90% confluent monolayer at a ratio of 1:3-1:4. For cell harvesting, the growth medium is removed from the monolayer and the cells are incubated with cell dissociation solution (Sigma) until showing signs of dissociation. The cells are subsequently knocked from the base of the flask and pelleted by centrifugation for storage (frozen at −80° C.) prior to further use.

ii) Cellular Membrane Preparation

Cell pellets are thawed on ice and resuspended in 3 mL of membrane preparation buffer (see Media and Buffers for composition) per 1 mL of packed cell volume, using a vortex mixer to disperse the cell pellet.
After incubation on ice for 10 minutes, the suspension is homogenised for four individual 10 second intervals using a hand-held homogeniser. The homogenate is then centrifuged at 1075×g for 20 minutes at 4° C.
The supernatants are then collected and retained. Initial cell & nuclei pellets (P1) are subsequently rehomogenised and centrifuged using the conditions cited above, and the supernatants collected and pooled with those retained from the first spin.

The pooled supernatants are centrifuged at 35000×g for 30 minutes at 4° C., and the supernatants discarded. The pellets (P2) are then resuspended in 1 mL of membrane preparation buffer per 1 mL of the original packed cell volume. Protein concentrations are then measured and the membrane suspension was finally frozen in aliquots of set volume and stored at −80° C. prior to use in assays.

iii) Assay Methodology

### A. Determination of Optimal Assay Conditions for Individual Membrane Batches

The specific SPA bead type differed for each transporter, wheat germ agglutinin-coated yttrium silicate (YSi WGA) SPA beads are used for hSERT and WGA-coated polyvinyltoluene (PvT WGA) SPA beads for hNET assays. For each batch of membrane used, optimal concentrations of bead and membrane are determined

Tritiated radioligands specific to each transporter (3H-citalopram for hSERT and ³H-nisoxetine for hNET) are used. The assay free radioligand concentration is expressed as a percentage of the total free radioligand concentration to give an estimate of the radioligand depletion. The radioligand depletion in assays for both transporters is less than 30% to ensure that there was sufficient radioligand available for binding. The ligand depletion value is also used for selecting the optimal assay conditions when using new batches of membranes.

The affinity of the specific radioligand for the respective transporter is determined for each membrane batch at the selected protein and bead concentrations. This is achieved by the determination of the K_{D}, the concentration of free radioligand at which 50% of the transporter binding sites were occupied. The mean K_{D} for a radioligand at a batch of membranes is determined from data from a minimum of three separate assays. The mean K_{D} is subsequently used for all assays using the membrane batch profiled to enable determination of Kᵢ values of compounds studied using the method determined by Cheng and Prussoff (Cheng Y C and Prusoff W H. Relationship between the inhibition constant (Kᵢ) and the concentration of inhibitor which causes 50% inhibition of an enzymatic reaction. Biochem Pharmacol 1973; 22:2099-3108.)

### B. Assay Protocol

### Bead/Membrane Complex Preparation

The required amount of membrane is thawed on ice and added to a predetermined volume of bead suspension in assay buffer. The beads are then pre-coupled by incubating the predetermined protein quantity per mg of bead on a shaker at a temperature of 4° C. for 2 hours. Subsequently, the bead/membrane complex is spun down at 865×g for 5 minutes. The resulting pellet is resuspended in assay buffer and this spin/wash step is then repeated. The final pellet is then resuspended in assay buffer at the specific concentration required for the final assay.

### Ligand Preparation

An aliquot of [³H]-radioligand stock is diluted in assay buffer to give a pre-determined final assay concentration less than the equilibrium dissociation constant (K_{D}) value.

### Compound Plate Preparation

All test compounds are prepared at a concentration of 4 mM in 100% dimethyl sulphoxide (DMSO) from dry samples. Compounds are diluted in 0.75% DMSO in ddH₂O to give appropriate test concentrations in a 384 well plate to give a final volume of 20 μL.

The same volume of assay buffer is added to specific wells of the plate to enable subsequent measurement of total radioligand binding. Furthermore, 20 μL a high concentration of compound specific to each transporter assay is subsequently added to predetermined wells to determine non-specific binding (NSB). Fluoxetine (10 μM final assay concentration) is used for hSERT and desipramine (40 μM final assay concentration) for hNET.

For each individual transporter assay, 20 μL of the prepared specific radioligand is added to each well of the final assay plates (containing compound solutions). Subsequently, 20 μL of the corresponding bead/membrane complex is added to each well of the final assay plate, ensuring that the suspension was mixed well. The plates are then sealed and incubated, with shaking, for 1 hour at room temperature. The plates are subsequently incubated for an additional 6 hours, with dark adaptation, prior to reading.

### C. Data Analysis

The assay window (specific binding) per plate is calculated by subtracting the mean NSB readings (in counts per minute, or cpm) from the mean of total binding readings. Subsequently the cpm read per well (with mean NSB subtracted) are expressed as a percentage of the plate window to determine the amount of radioligand bound to the transporter.

These values are plotted against the concentration of the compound tested and a sigmoidal inhibitory concentration effect curve was fitted to the data using a four-parameter logisitic equation and free-fitting parameters to give an IC₅₀ value (the concentration of compound required to inhibit 50% of the specific binding at the neurotransmitter transporter).

The inhibitory dissociation constant (K_{I}) value is then calculated from the IC₅₀ value using the Cheng-Prusoff equation

Following determination of individual Kᵢ values for compounds tested, an overall geometric mean is calculated together with 95% confidence intervals and n values, where n is the total number of individual Kᵢ values.

iv) Media and Buffers

hSERT Cell Growth Medium
DMEM, 10% (w/v) dialysed FCS
2 mM L-glutamine (diluted from 200 mM stock)
25 mM HEPES (diluted from 1 M stock)
250 μg/mL genetecin
hNET Cell Growth Medium
DMEM, 10% (w/v) FCS
2 mM L-glutamine (diluted from 200 mM stock)
25 mM HEPES (diluted from 1 M stock)
250 μg/mL genetecin

### Membrane Preparation Buffer

20 mM HEPES (diluted from 1M stock with ddH₂O), pH 7.4 at room temperature, stored at 4° C. Prior to use, one complete protease inhibitor tablet is dissolved per 50 mL of buffer.

### Assay Buffer (1.5×Final Assay Concentration)

30 mM HEPES (diluted from 1 M stock with ddH₂O) and 180 mM NaCl (diluted from 5 M stock with ddH₂O), pH 7.4 at room temperature, stored at 4° C.

The compounds can also be tested in specific disease models, such as the pain models as follows:

### Neuropathic Pain

The activity of a compound in the treatment of neuropathic pain may be measured according to the following test protocol.

Animals: Male Sprague Dawley rats are housed in appropriately sized groups. All animals are kept under a 12 h light/dark cycle (lights on at 07 h 00 min) with food and water ad libitum. All experiments are carried out by an observer blind to the treatments and in accordance with the Home Office Animals (Scientific Procedures) Act 1986.

Chronic Constriction Injury (CCI) Rat Model of Neuropathic Pain

The CCI of sciatic nerve is performed as previously described by Bennett and Xie (Bennett G J, Xie Y K. A peripheral mononeuropathy in rat that produces disorders of pain sensation like those seen in man. Pain: 33:87-107, 1988). Animals are anaesthetised with a 2% isofluorane/O2 mixture. The right hind thigh is shaved and swabbed with 1% iodine. Animals are then transferred to a homeothermic blanket for the duration of the procedure and anaesthesia maintained during surgery-via a nose cone. The skin is cut along the line of the thighbone. The common sciatic nerve is exposed at the middle of the thigh by blunt dissection through biceps femoris. About 7 mm of nerve is freed proximal to the sciatic trifurcation, by inserting forceps under the nerve and the nerve gently lifted out of the thigh. Suture is pulled under the nerve using forceps and tied in a simple knot until slight resistance is felt and then double knotted. The procedure is repeated until 4 ligatures (4-0 silk) are tied loosely around the nerve with approx 1 mm spacing. The incision is closed in layers and the wound treated with topical antibiotics.

### Streptozocin (STZ)-Induced Diabetes Neuropathy in the Rat

Diabetes is induced by a single intraperitoneal injection of streptozotocin (50 mg/kg) freshly dissolved in 0.9% sterile saline. Streptozotocin injection induces a reproducible mechanical allodynia within 3 weeks, lasting for at least 7 weeks (Chen and Pan, (Chen S R and Pan H L. Hypersensitivity of Spinothalamic Tract Neurons Associated With Diabetic Neuropathic Pain in Rats. J Neurophysiol 87:2726-2733, 2002).

### Assessment of Static and Dynamic Allodynia

### Static Allodynia.

Animals are habituated to wire bottom test cages prior to the assessment of allodynia. Static allodynia is evaluated by application of von Frey hairs (Stoelting, Wood Dale, Ill., USA.) in ascending order of force (0.6, 1, 1.4, 2, 4, 6, 8, 10, 15 and 26 grams) to the plantar surface of hind paws. Each von Frey hair is applied to the paw for a maximum of 6 sec, or until a withdrawal response occurred. Once a withdrawal response to a von Frey hair is established, the paw is re-tested, starting with the filament below the one that produced a withdrawal, and subsequently with the remaining filaments in descending force sequence until no withdrawal occurs. The highest force of 26 g lifts the paw as well as eliciting a response, thus represented the cut off point. Each animal has both hind paws tested in this manner. The lowest amount of force required to elicit a response is recorded as paw withdrawal threshold (PWT) in grams. Static allodynia is defined as present if animals respond to a stimulus of, or less than, 4 g, which is innocuous in naive rats (Field M J, Bramwell S, Hughes J, Singh L. Detection of static and dynamic components of mechanical allodynia in rat models of neuropathic pain: are they signalled by distinct primary sensory neurones Pain, 1999; 83:303-11).

### Dynamic Allodynia

Dynamic allodynia is assessed by lightly stroking the plantar surface of the hind paw with a cotton bud. To avoid recording general motor activity, care is taken to perform this procedure in fully habituated rats that are not active. At least two measurements are taken at each time point, the mean of which represents the paw withdrawal latency (PWL). If no reaction is exhibited within 15 sec the procedure is terminated and animals are assigned this withdrawal time. A pain withdrawal response is often accompanied with repeated flinching or licking of the paw. Dynamic allodynia is considered to be present if animals respond to the cotton stimulus within 8 sec of commencing stroking (Field et al, 1999).

### Nociceptive Pain

The activity of a compound in the treatment of nociceptive pain may be measured according to the following test protocols.

### Hotplate

Experimental Procedure: Male Sprague Dawley rats are placed on a hot plate (Ugo Basile, Italy) maintained at 55±5° C. The time between placement of the animal on the hot plate and occurrence of either licking of fore or hind paw, shaking or jumping off the surface is measured. Baseline measurements are made and animals reassessed following drug administration. The cut off time for hot plate latencies is set at 20 seconds to prevent tissue damage.

### Ovariohysterectomy (OVX)

Experimental Procedure: Female Sprague Dawley rats are placed into an anaesthetic chamber and anaesthetised with a 2% isofluorane O₂ mixture. During surgery, anaesthesia is maintained via a nose cone. OVX is performed via a midline incision (2 cm in length) in the linea alba, whilst the animal is on a heat blanket. The ovarian ligaments and cervix are ligated with 5-0 silk, using a single clamp technique. The ovaries and uterus are then removed. The abdominal wall is closed using 4 simple interrupted sutures and the skin closed using 4 wound clips. Immediately after surgery animals are placed in individual plexiglass chambers. Once the animal has recovered from the anaesthetic the abdominal body postures are recorded in 30 min bins at various time points. Postures scored are humpback position, contraction of the muscle of the abdomen associated with inward movements of the hind limb, stretching of the body and squashing of the lower abdomen against the floor. Each of these behaviours is scored as one posture.

### Brennan

Experimental Procedure: Male Sprague Dawley rats are placed into an anaesthetic chamber and anaesthetised with a 2% isofluorane O₂ mixture. During surgery, anaesthesia is maintained via a nose cone. The plantar aspect of the right hind paw is cleaned with 50% ethanol. A 1 cm long longitudinal incision is made with a number 11 blade through the skin and fascia of the plantar aspect of the foot, starting 0.5 cm from the proximal edge of the heel and extending toward the toes. The plantaris muscle is elevated using forceps and incised longitudinally, the muscle origin and insertion remain intact. After haemostasis with gentle pressure, the skin is closed with two simple sutures of braided silk.

### Mono-Iodoacetate (MIA)-Induced OA Model

Male 6 weeks-old Sprague-Dawley (SD, Japan SLC or Charles River Japan) rats are anesthetized with pentobarbital. Injection site is shaved and cleaned with 70% ethanol. 25 μl of MIA solution or saline is injected in the right knee joint using a 29 G needle. 7,14,19 and 20 days after the MIA injection, train rats to measure the weight bearing (WB) without their stress. 21 days after the MIA injection, the WB on two of each hind paw is measured and the WB deficit is calculated. Define the WB deficit value as “pre value”. Arrange for experimental group evenly in consideration of pre value and prepre value. After the administration of test compounds or vehicle, the WB on two of each hind paw was measured.

### Cancer Pain Model

These experiments use adult male C3H/HeN mice (Nihon SLC, Shizuoka, Japan). The mice are housed in accordance with National Institutes of Health guidelines in a vivarium maintained at 22° C. with a 12-hour alternating light-dark cycle, and were given food and water ad libitum. The sarcoma injection protocol which is used has been described. After induction of general anesthesia with an inhalation of isofluran (2%), a superficial incision is made in the skin overlying the patella, using Mora scissors. The patellar ligament is then cut, exposing the condyles of the distal femur. A 30-gauge needle is inserted at the level of the intercondylar notch and into the medullary canal to create an initial core pathway. After the initial core is made, a 29-gauge needle is used to make the final pathway into the bone. A 0.5-mm depression is then made using a half-round bur in a pneumatic dental high speed handpiece, to serve as mechanical retention for the dental resin plug. Then, 20μ|α-minimum essential media (Sigma; sham injection) or 20H media containing 1×10⁵ 2472 osteolytic sarcoma cells (American Type Culture Collection, Rockville, Md.; sarcoma injection) is injected using a 29-gauge needle and a 0.25 cc syringe. To prevent leakage of cells outside the bone, the injection site is closed with dental resin, followed by copious irrigation with filtered water. Wound closure is achieved using auto wound clips (Becton Dickinson, San Jose, Calif.). Wound clips are removed at day 5 to prevent interference with behavioral testing.

### Assessment of Static and Dynamic Allodynia

### Static Allodynia.

Animals are habituated to wire bottom test cages prior to the assessment of allodynia. Static allodynia is evaluated by application of von Frey hairs (Stoelting, Wood Dale, Ill., USA.) in ascending order of force (0.6, 1, 1.4, 2, 4, 6, 8, 10, 15 and 26 grams) to the plantar surface of hind paws. Each von Frey hair is applied to the paw for a maximum of 6 sec, or until a withdrawal response occurs. Once a withdrawal response to a von Frey hair is established, the paw is re-tested, starting with the filament below the one that produces a withdrawal, and subsequently with the remaining filaments in descending force sequence until no withdrawal occurs. The highest force of 26 g lifts the paw as well as eliciting a response, thus represents the cut off point. Each animal has both hind paws tested in this manner. The lowest amount of force required to elicit a response is recorded as paw withdrawal threshold (PWT) in grams. Static allodynia is defined as present if animals respond to a stimulus of, or less than, 4 g, which is innocuous in naive rats (Field M J, Bramwell S, Hughes J, Singh L. Detection of static and dynamic components of mechanical allodynia in rat models of neuropathic pain: are they signalled by distinct primary sensory neurones? Pain, 1999, 83:303-11).

### Dynamic Allodynia

Dynamic allodynia is assessed by lightly stroking the plantar surface of the hind paw with a cotton bud. To avoid recording general motor activity, care is taken to perform this procedure in fully habituated rats that are not active. At least two measurements are taken at each time point, the mean of which represents the paw withdrawal latency (PWL). If no reaction is exhibited within 15 sec the procedure is terminated and animals are assigned this withdrawal time. A pain withdrawal response is often accompanied with repeated flinching or licking of the paw. Dynamic allodynia is considered to be present if animals respond to the cotton stimulus within 8 sec of commencing stroking (Field et al, 1999).

### Radiant Heat Paw Withdrawal

Experimental procedure: Thermal paw withdrawal is assessed using the rat plantar test (Ugo Basile, Italy) following a modified method of Hargreaves et al., 1988. Rats are habituated to the apparatus that consists of three individual perspex boxes on an elevated glass table. A mobile radiant heat source is located under the table and focused onto the hind paw and paw withdrawal latencies (PWL) are recorded. There is an automatic cut off point of 22.5 s to prevent tissue damage. PWL are taken 2-3 times for both hind paws of each animal, the mean of which represents baselines for right and left hind paws. The apparatus is calibrated to give a PWL of approximately 10 s.

### Weight Bearing

Experimental procedure: Animals are examined for hypersensitivity in the weight-bearing test, using an “incapacitance tester” (Linton Instruments, Diss, Norfolk, U.K.). Rats were positioned with their fore limbs up on a perspex slope and hind limb weight distribution was measured via force transducers under each of the hind paws. Each animal is placed in the apparatus and the weight load exerted by the hind paws is noted. The difference in weight bearing is calculated by subtracting the ipsilateral (injured) paw from the contralateral paw (normal) and this constitutes the raw data.

### Inflammatory Pain

The activity of compound in the treatment of inflammatory pain may be measured according to the following test protocol.

### CFA-Induced Weight Bearing Deficits in Rats

Male 7-week-old SD rats are fasted overnight. CFA (300 μg of Mycobacterium Tuberculosis H37 RA (Difco Laboratories) in 100 μL of liquid paraffin (Wako)) is injected into the rat's right hind footpad. Two days after the administration of CFA, the changes in hind paw weight distribution between the left (ipsilateral) and the right (contralateral) limbs are measured as an index of pain by using Linton Incapacitance tester (Linton Instrumentation, UK). The test compound suspended in 0.1% MC (Wako) is administered orally in a volume of 1 mL per 100 g body weight. Each animal is placed in the apparatus and the weight load exerted by the hind paws is measured before, 1, 2 and 4 hours after drug administration.

### Carrageenin-Induced Mechanical Hyperalgesia in Rats

Male 4-week-old SD rats are fasted overnight. Hyperalgesia is induced by intraplantar injection of Lambdā-carrageenin (0.1 ml of 1% w/v solution in saline, Zushikagaku). The test compound (1 ml of 0.1% methylcellulose/100 g body weight) is given orally at 5.5 hours after the carrageenin injection. The paw withdrawal threshold (gram) is measured by analgesimeter (Ugo Basile) at 3.5, 4.5, 6.5 and 7.5 hours after the carrageenin injection. (Randall L. O. & Selitto I. J., Arch. Int. Pharmacodyn. 111, 409-419, 1957)

### Carrageenan-Induced Thermal Hyperalgesia (CITH) in the Rat

Thermal hyperalgesia is assessed using the rat plantar test (Ugo Basile, Comerio, Italy), according to a method modified by Hargreaves et al. (1988). Briefly, rats are habituated to the apparatus that consists of three individual Perspex boxes on a glass table. A mobile radiant heat source is located under the table and focused onto the desired paw. Paw withdrawal latencies (PWLs) are recorded three times for both hind paws of each animal, the mean of which represents baseline for left and right hind paws. The apparatus is calibrated to give a PWL of approximately 10 s in naive rats. To prevent tissue damage of the plantar zone, a 22.5 sec cut-off is observed. Lambda carrageenan is injected intraplantarly (100 μl, 20 mg/ml) the right hind paw and baseline recordings of PWT are taken 2 hr post administration.

### Visceral Pain

The activity of a compound in the treatment of visceral pain may be measured according to the following test protocols.

Several models are available to determine if a compound is effective in treating disorders of the viscera. These models include a LPS model (Eutamene H et al, J Pharmacol Exp Ther 2000 295 (1): 162-7), a TNBS model (Diop L. et al, Gastroenterology 1999, 116, 4(2): A986), a IBD model (Clemett D, Markham A, Drugs 2000 April; 59(4):929-56), a pancreatic pain model (Isla A M, Hosp Med 2000 June; 61(6):386-9) and a visceral non digestive pain model (Boucher M et al, J Urol 2000 July; 164(1):203-8).

### TNBS-Induced Chronic Visceral Allodynia in Rats

In this experimental model of colonic distension in awake rats, previous injection of trinitrobenzenesulfonic acid (TNBS) into the proximal colon lowered the visceral pain threshold.

Materials and methods: Male Sprague-Dawley rats are used. The animals are housed 3 per cage in a regulated environment (20±1° C., 50±5% humidity, with light 8:00 am to 8:00 pm). At day 0, under anesthesia (ketamine 80 mg/kg i.p.; acepromazine 12 mg/kg i.p.), the injection of TNBS (50 mg/kg in ethanol 30%), or saline (1.5 ml/kg) for control rats, is performed into the proximal colon wall (1 cm from the cecum). After the surgery, animals are individually housed in polypropylene cages and kept in a regulated environment (20±1° C., 50±±5% humidity, with light 8:00 a.m. to 8:00 p.m.) during 7 days. At day 7 after TNBS administration, a balloon (5-6 cm length) is inserted by anus, and kept in position (tip of balloon 5 cm from the anus) by taping the catheter to the base of the tail. Oral administration of the test compound is performed 1 h before the colonic distension cycle: the balloon is progressively inflated by steps of 5 mm Hg (0.667 kPa), from 0 to 75 mm Hg, each step of inflation lasting 30 s. Each cycle of colonic distension is controlled by a standard barostat. The threshold (mm Hg) corresponds to the pressure which produced the first abdominal contraction, and the cycle of distension is then discontinued. The colonic threshold is determined after performance of four cycles of distension on the same animal.

### LPS-Induced Rectal Hypersensitivity in Rats

Intraperitoneal injection of bacterial lipo-polysaccharide (LPS) has been shown to induce rectal hyperalgesia in awake rats.

Materials and methods: Animals are surgically prepared for electromyography: rats are anaesthetized by intraperitoneal injection of acepromazine (0.6 mg/kg) and ketamine (120 mg/kg). Three groups of three electrodes are implanted in the abdominal external oblique musculature, just superior to the inguinal ligament. Electrodes are exteriorized on the back of the neck and protected by a glass tube attached to the skin. Animals are individually housed in polypropylene cages and kept in a temperature-controlled room (21° C.). Food (UAR pellets, Epinay, France) and water are provided ad libitum.

Electromyographic recordings begin five days after surgery. The electrical activity of abdominal striated muscles is recorded with an electroencephalograph machine (Mini VIII Alvar, Paris, France) using a short time constant (0.03 s) to remove low-frequency signals (< 3 Hz) and a paper speed of 3.6 cm/min. Spike bursts are recorded as an index of abdominal contractions.

Distension procedure: Rats are placed in plastic tunnels (6 cm diameter×25 cm long), where they cannot move, escape, or turn around, in order to prevent damage to the balloon. Animals are accustomed to this procedure for four days before rectal distension in order to minimize stress reactions during experiments. The balloon used for distension is an arterial embolectomy catheter (Fogarty, Edwards Laboratories Inc.). Rectal distension is performed by insertion of the balloon (2 mm diameter×2 cm long) into the rectum, at 1 cm from the anus, and catheter is fixed at the base of the tail. It is inflated progressively with tepid water by steps of 0.4 ml, from 0 to 1.2 ml, each step of inflation lasting 5 min. To detect possible leakage, the volume of water introduced in the balloon is checked by complete removal with a syringe at the end of the distension period.

1-22. (canceled)

23. A compound of Formula (I) 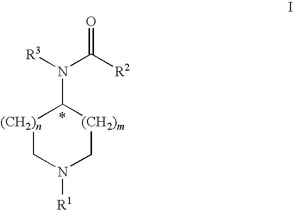
and pharmaceutically and/or veterinarily acceptable derivatives thereof, wherein:
R¹ is H, C₁₋₆alkyl, —C(A)D, C₃₋₈cycloalkyl, aryl, het, aryl-C₁₋₄alkyl or het-C₁₋₄alkyl, wherein the cycloalkyl, aryl or het groups are optionally substituted by at least one substituent independently selected from C₁₋₈allkyl, C₁₋₈alkoxy, OH, halo, CF₃, OCHF₂, OCF3, SCF₃, hydroxy-C₁₋₆alkyl, C₁₋₄alkoxy-C₁₋₆alkyl and C₁₋₄alkyl-S—C₁₋₄alkyl;
A is S or O;
D is H, C₁₋₆alkyl, aryl, het, aryl-Cl₁₋₄alkyl or het-C₁₋₄alkyl;
at each occurrence aryl independently represents phenyl, naphthyl, anthracyl or phenanthryl;
at each occurrence het independently represents an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom;
R² represents aryl¹ or het¹, each of which is substituted by at least one substituent independently selected from B, provided that when R² is substituted by halo then it is also substituted with at least one other substituent independently selected from B other than halo;
aryl¹ is selected from phenyl, naphthyl, anthracyl and phenanthryl;
het¹ represents an aromatic 5- or 6-membered heterocycle which contains at least one N, O or S heteroatom, optionally fused to an aryl group;
B represents aryl², het², Oaryl², Ohet², Saryl², Shet², SC₁₋₆alkyl, halogen, CHF₂, OCHF₂, CF₂CF₃, CH₂CF₃, CF₂CH₃, aryl²-C₁₋₄alkyl, C₃₋₆cycloalkyl, C₃₋₆cycloalkyl-C₁₋₄alkyl, C₃₋₆cycloalkyl-C₁₋₄alkoxy, C₃₋₆cycloalkyl-O—C₁₋₄alkyl, C₃₋₆cycloalkyl-C₁₋₄alkoxy-C₁₋₄alkyl, OC₃₋₆cycloalkyl, SC₃₋₆cycloalkyl; wherein the aryl² and het² groups are optionally substituted by at least one group selected from C₁₋₆alkyl, C₃₋₆cycloalkyl, C₁₋₄alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃;
at each occurrence, aryl² independently represents phenyl, naphthyl, anthracyl or phenanthryl;
at each occurrence, het² independently represents an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom;
R³ is H, C₁₋₆alkyl, C₃₋₈cycloalkyl, C₃₋₈cycloalkyl-C₁₋₆alkyl, aryl³, het³, aryl³-C₁₋₄alkyl or het³-C₁₋₄alkyl, wherein the C₃₋₈cycloalkyl, aryl³ or het³ groups are optionally substituted by at least one substituent independently selected from C₁₋₆alkyl, C₁₋₆alkoxy, CN, OH, halo, CF₃, OCF₃, SCF₃, hydroxy-C₁₋₆alkyl, C₁₋₄alkoxy-C₁₋₆alkyl and C₁₋₄alkyl-S—C₁₋₄alkyl; aryl³ represents phenyl, naphthyl, anthracyl or phenanthryl; and
het³ represents an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom.

24. A compound according to claim 23, wherein R¹ is H.

25. A compound according to claim 23, wherein m is 0 and * represents the R or S enantiomer.

26. A compound according to claim 25, wherein * represents the S enantiomer.

27. A compound according to claim 23, wherein R² is selected from phenyl, naphthyl, pyridinyl and quinolinyl, each substituted by at least one substituent independently selected from B.

28. A compound according to claim 23, wherein B represents Oaryl², SC₁₋₆alkyl, Saryl², C₁₋₄alkyl-aryl², halogen, OCHF₂, CF₂CH₃, C₃₋₆cycloalkyl, and C₃₋₆cycloalkyl-C₁₋₄alkyloxy, wherein the aryl² group is independently optionally substituted by at least one group independently selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃.

29. A compound according to claim 23, wherein R³ is other than H.

30. A pharmaceutical composition comprising a compound as claimed in claim 23 and a pharmaceutically acceptable adjuvant, diluent or carrier.

31. A method of treatment of a disorder in which the regulation of monoamine transporter function is implicated which comprises administering a therapeutically effective amount of a compound according to claim 23 to a patient in need of such treatment.

32. A method of treatment of a disorder in which the regulation of serotonin or noradrenaline is implicated which comprises administering a therapeutically effective amount of a compound according to claim 23 to a patient in need of such treatment.

33. A method according to claim 32, wherein the regulation of serotonin and noradrenaline is implicated.

34. A method of treatment of urinary disorders, depression, pain, premature ejaculation, hot flashes, ADHD or fibromyalgia, which comprises administering a therapeutically effective amount of a compound according to claim 23 to a patient in need of such treatment.

35. A method according to claim 34, wherein the urinary disorder is urinary incontinence, such as GSI or USI.

36. A method according to claim 34, wherein pain is treated.

37. A process for preparing a compound according to claim 23 comprising reacting a compound of formula X: 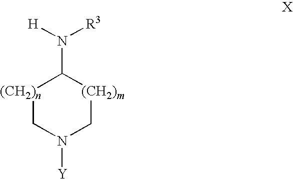
wherein R³, n and m are as defined in claim 23 and Y is R¹ or a protecting group, with an acid or acyl halide: R²COX, wherein X is OH or halo, or an acid anhydride, and, optionally, deprotecting.
